(19) United States
(12) Patent Application Publication
(10) Pub. No.: US 2026/0094720 A1
(43) Pub. Date: Apr. 2, 2026

(54) MONITORING TOOLS AND DIAGNOSTIC METHODS

(71) Applicant: MARS, INCORPORATED, McLean, VA (US)

(72) Inventors: Zoe V. MARSHALL-JONES, Melton Mowbray (GB); Ruth STAUNTON, Melton Mowbray, (GB); Richard HAYDOCK, Melton Mowbray (GB); Ciaran O'FLYNN, Melton Mowbray (GB); Phillip WATSON, Melton Mowbray (GB)

(73) Assignee: MARS, INCORPORATED, McLean, VA (US)

(21) Appl. No.: 19/307,965

(22) Filed: Aug. 22, 2025

Related U.S. Application Data
(63) Continuation of application No. 17/423,708, now abandoned.

(30) Foreign Application Priority Data
Jan. 18, 2019 (GB) 1900745.9

(51) Int. Cl. G16H 50/30; C12Q 1/6869; C12Q 1/689; G16H 20/60

(52) U.S. Cl. CPC G16H 50/30; C12Q 1/6869; C12Q 1/689; G16H 20/60; C12Q 2600/124

(57) ABSTRACT

Methods for assessing a feline's microbiome health are provided. The methods include, inter alia, determining the health of a feline's microbiome comprising quantitation four or more bacterial species and determining the relative abundance of the four or more bacterial species by comparing the abundance with a control data set, wherein an increase or decrease in the abundance can be indicative of an unhealthy microbiome.

### CROSS-REFERENCE TO RELATED APPLICATIONS

This application is a continuation of U.S. patent application Ser. No. 17/423,708, filed on Jul. 16, 2021, which is a National Stage Entry of International Patent Application No. PCT/US2020/014303, filed on Jan. 20, 2020, which claims the benefit of priority to UK Patent Application No. 1900745.9, filed on Jan. 18, 2019, the contents of each of which are hereby incorporated herein by reference in their entireties, and to each of which priority is claimed.

### SEQUENCE LISTING

The instant application contains a Sequence Listing which has been submitted electronically in XML format and is hereby incorporated by reference in its entirety. Said XML copy, created on Aug. 12, 2025, is named 069269_0753_SL.xml and is 100,859 bytes in size. The Sequence Listing, electronically filed herewith, does not extend beyond the scope of the specification and thus does not contain new matter.

### TECHNICAL FIELD

This present disclosure relates to the field of monitoring tools and diagnostic methods for determining the health of a feline's microbiome.

### BACKGROUND TO THE INVENTION

The understanding of the microbiome and its impact on health has increased significantly in recent years. Changes in the microbiome, and its interaction with the immune, endocrine and nervous systems are correlated with a wide array of illnesses, ranging from inflammatory bowel disease to cancer [4] and to behavioral aspects of host health [5;6].

Despite key differences in nutritional intake, such as the carnivorous diet of the domestic cat (Felis catus), the gastrointestinal microbiota of cats and dogs is, similarly to humans, a highly complex ecosystem consisting of several hundred different bacterial taxa with the Firmicutes, Bacteriodetes, Proteobacteria, Fusobacteria and Actinobacteria representing the dominant phyla [7]. Reference 7 (Handl et al.) describes that in healthy animals, similarly to human hosts, the faecal bacterial community is highly diverse in both dogs and cats, with 85 and 113 operational taxonomic units (OTUs) identified in the two species respectively. The specific taxonomic composition of the microbiota however can be relatively unique to the host or at least distinct between different species or mammals with diverse nutritional requirements [8].

Assessment of the microbiota in health and disease, particularly in the case of clinical signs such as chronic and acute diarrhoca and gastrointestinal (GI) inflammatory conditions, have represented a key focus in human and pet health research. As a result of this focus, many gastrointestinal conditions previously designated infections are now considered concurrent with a more general dysbiosis in the microbiota, with consequences on the whole microbiome rather than resulting from a single organism infecting the gastrointestinal mucosa [9;10].

Dysbiosis, the loss of constituents of the normal commensal flora (e.g., Lachnospiraceae, Ruminococcaceae and Faecalibacterium spp.) can occur in acute and chronic intestinal diseases and has been linked to changes in immunomodulatory bacterial metabolites such as short chain fatty acids and secondary bile acids [11]. In a study of cats with chronic and acute diarrheal disease, significant differences were identified in bacterial groups between healthy cats and cats with diarrhea.

Studies to assess the intestinal microbiota throughout life in cats have, similarly to those in the human host, demonstrated significant changes in taxonomic groups associated with the aging process either during development of the mature microflora or in the senior/geriatric life stage. In a post-weaning study of the development of the microbiota in kittens aged 8-16 weeks the authors found lower than expected levels of taxonomic succession [12]. Only 18 bacterial genera and 11 families showed significant differences in relative abundance over time. No significant differences in the Shannon diversity were identified over the period of 8-16 weeks. These findings can suggest that the establishment of the feline microbiota occurs in the first weeks of life prior to the initiation of the study. Denaturing gradient gel electrophoresis (DGGE) revealed that unlike in the developing microbiota of the human host, microbial profiles were more diverse in kittens prior to weaning than they were in older kittens with a more mature microbiota and the variability of the microbiota was also higher in 4-week old kittens compared to post-weaning.

At the other end of the aging spectrum in older and geriatric cats several studies have assessed components of the gastrointestinal microbiota. Aging is associated with an increased incidence of GI pathologies including infection, neoplasia, or other inflammatory conditions. Reported physiological alterations in digestive function associated with advancing age includes slower GI transit, altered enzymatic activity and reduced bile secretions [13]. Histological changes also occur in the gut with aging including reduced duodenal villus surface area, lower jejunal villus height, and greater colonic crypt depth [14]. Whether the full range of age-related changes in

digestion and absorption of nutrients recognized in humans also affects pet animals remains unclear.

Similarly to the understanding of gastrointestinal physiology in aging, human research conducted over the last decade has uncovered associations between aging and alterations in the gut microflora. More recently high-throughput sequencing (HTS) and specialised DNA array technologies have yielded further evidence of links between the microbiome and healthy longevity.

To date, research into the enhancement of health through the feline gastrointestinal microbiome has largely focussed on organisms influencing human health, and those of production animals. The degree to which these taxa are directly transferable to feline gastrointestinal (GI) health remains unclear. However, understanding of the faecal microbiome in healthy cats throughout the various life stages associated with changes in digestive function and relative resilience to infection and diarrhoeal incidence, represents a first step towards identifying putative feline specific markers of gastrointestinal health in cats throughout life.

Several publications have discussed the composition of the microbiome in healthy felines and some of these have suggested that the microbiome can change in disease [16-19]. However, the authors speculate inter alia that these differences could have been caused by differences in dietary factors and cat breeds, and none of these studies takes into account the life stage of the cats. Given the importance of the microbiome to health and wellbeing, it is important to find ways to determine the health of the microbiome of a feline.

### SUMMARY OF THE INVENTION

The presently disclosed subject matter has developed methods which allow the determination of the health of a feline's microbiome. The methods of the present disclosure can achieve this with high accuracy, as shown in the Examples.

In one aspect, the disclosure features a method of determining the health of a feline's microbiome, comprising quantitating four or more bacterial species in a sample obtained from the feline to determine their relative abundance; and comparing the relative abundance to the relative abundance of the same species in a control data set; wherein an increase or decrease in the abundance of the four or more bacterial species relative to the control data set is indicative of an unhealthy microbiome. As discussed above, an unhealthy microbiome is associated with a number of pathological conditions and it is therefore desirable to diagnose an unhealthy microbiome.

In another aspect, the present disclosure features a method of determining the health of a feline's microbiome, the method comprising the steps of calculating the diversity index for the species within the feline's microbiome and comparing the diversity index to the diversity index of a control data set. In certain embodiments, the diversity index is the Shannon Diversity Index.

In another aspect, the disclosure also features a method of monitoring a feline, comprising a step of determining the health of the feline's microbiome by a method of the present disclosure on at least two time points. This is particularly useful where a feline is receiving treatment to shift the microbiome as it can monitor the progress of the therapy. It is also useful for monitoring the health of the feline.

In some embodiments, the methods of the present disclosure comprise a further step of changing the composition of the microbiome. This can be achieved through a dietary change or through administration of a nutraceutical or pharmaceutical composition comprising bacteria. This will usually be done where the microbiome is deemed unhealthy but can also be undertaken pre-emptively.

In another aspect, also provided is a method of monitoring the health of the microbiome in a feline who has undergone a dietary change or who has received a nutraceutical or pharmaceutical composition which is able to change the microbiome composition, comprising determining the health of the microbiome by a method according to the present disclosure. Such methods allow a skilled person to determine the success of the treatment. In certain embodiments, these methods comprise determining the health of the microbiome before and after treatment as this helps to evaluate the success of the treatment.

As noted above, the presently disclosed subject matter provides a method of determining the health of a feline's microbiome, comprising quantitating four or more bacterial species in a sample obtained from the feline to determine their relative abundance; and comparing the relative abundance to the relative abundance of the same species in a control data set; wherein an increase or decrease in the abundance of the four or more bacterial species relative to the control data set is indicative of an unhealthy microbiome. In a particular embodiment of this method, the bacterial species are from genera or families selected from the group consisting of [Eubacterium], [Eubacterium] hallii group, Anaerobiospirillum, Anaerostipes, Anaerotruncus, Bifidobacterium, Blautia, Butyricicoccus, Catenibacterium, Clostridium sensu stricto 1, Collinsella, Coriobacteriaceae, Faecalibacterium, Holdemanella, Lachnoclostridium, Lachnospiraceae, Lachnospiraceae [Eubacterium] hallii group, Lachnospiraceae [Ruminococcus] gauvreauii, Lachnospiraceae FCS020 group, Lachnospiraceae genus, Lachnospiraceae NK4A136 group, Lactobacillus, Megamonas, Megasphaera, Peptoclostridium [Clostridium], Romboutsia, Roseburia, Ruminococcaceae, Ruminococcaceae UCG-009, Sarcina, Sellimonas, Subdoligranulum, Succinivibrio, and Turicibacter. In another embodiment of this method, the bacterial species are selected from the group consisting of [Clostridium] hiranonis, [Eubacterium] brachy, [Eubacterium] hallii group sp., Anaerobiospirillum succiniciproducens, Anaerostipes sp., Anaerotruncus sp., Bifidobacterium sp., Bifidobacterium saeculare, Blautia [Ruminococcus] gnavus, Blautia sp., Butyricicoccus sp., Catenibacterium sp., Clostridium sensu stricto 1 sp., Collinsella sp., Coriobacteriaceae UCG-002/UCG-003, Faecalibacterium sp., Holdemanella sp., Lachnoclostridium sp., Lachnospiraceae sp., Lachnospiraceae [Eubacterium] hallii group sp., Lachnospiraceae [Ruminococcus] gauvreauii group sp., Lachnospiraceae [Ruminococcus] gauvreauii group sp., Lachnospiraceae FCS020 group sp., Lachnospiraceae genus sp., Lachnospiraceae NK4A136 group sp., Lactobacillus sp., Megamonas sp., Megasphaera sp., Romboutsia sp., Roseburia sp., Ruminococcaceae sp., Sarcina sp., Sellimonas sp., Subdoligranulum sp., Succinivibrio sp., Turicibacter sp., and UCG-009 sp.

In certain embodiments of the claimed methods, the bacterial taxa have a 16S rDNA sequence with at least about 95%, at least about 96%, at least about 97%, at least about 98%, at least about 99% or 100% identity to the sequence of any one of the sequences selected from the group consisting of SEQ ID NOs 3-73.

In certain embodiments of the claimed methods, the control data set comprises microbiome data of a feline at the same life stage. In certain embodiments of the claimed methods, the feline is a kitten, an adult, a senior or a geriatric feline.

In an alternative embodiment, the disclosed subject matter provides a method of determining the health of a feline's microbiome, comprising calculating the diversity index for the species within the feline's microbiome and comparing the diversity index to the diversity index of a control data set. In particular embodiments of the claimed method, the feline is an adult and the microbiome is considered healthy if the diversity index falls in the range of from about 2.0 to about 4,5, or in the range of about 3.14 to about 3.60. In particular embodiments of the claimed method, the feline is a senior and the microbiome is considered healthy if the diversity index falls in the range of from about 2.41 to about 3.92, or in the range of from about 2.93 to about 3.40. In particular embodiments of the claimed method, the feline is geriatric and the microbiome is considered healthy if the diversity index falls in the range of from about 1.65 to about 4.17, or in the range of from about 2.51 to about 3.254. In particular embodiments of the claimed method, the diversity index is the Shannon diversity index.

The presently disclosed subject matter also provides a method of monitoring a feline, comprising a step of determining the health of the feline's microbiome by the method of any preceding claim on at least two time points. In certain embodiments of the claimed method, the two time points are at least about 6 months apart.

In certain embodiments of the claimed subject matter, the sample is from the gastrointestinal tract. In certain embodiments of the claimed subject matter, the sample is a faecal sample.

In an alternative embodiment, the disclosed subject matter provides a method of changing the microbiome composition of a feline, comprising (a) a step of determining the health of the feline's microbiome by a method of any preceding claim and (b) changing the microbiome of the feline. In certain embodiments of the claimed method, the feline has an unhealthy microbiome. In certain embodiments of the claimed method, the step (b) comprises changing the diet of the feline and/or administering a supplement or functional food or a pharmaceutical composition or a nutraceutical composition to the feline.

In an alternative embodiment, the disclosed subject matter provides a method of monitoring the microbiome health in a feline who has undergone a diet change and/or has received a supplement or functional food or a pharmaceutical or nutraceutical composition which is able to change the microbiome composition, comprising determining the health of the microbiome by the method of any preceding claim. In certain embodiments of the claimed method, the health of the microbiome is determined before and after diet change and/or administration of the supplement or functional food or pharmaceutical or nutraceutical composition. In certain embodiments of the claimed method, the supplement or functional food or nutraceutical composition or pharmaceutical composition comprises bacteria.

In certain embodiments of the claimed methods, the feline is a cat.

### BRIEF DESCRIPTION OF DRAWINGS

FIG. 1: Shannon diversity of the faecal microbiota in adult cats by life stage group (* p=0.007).

FIG. 2 corresponds to Table 1.1, which provides bacterial taxa (OTUs) associated with health in mammals and with utility for the detection of health in cats. Tables 1.1 (i)-(viii) each correspond to a section of Table 1.1, which are joined together as shown in the figure to form the full Table 1.1.

FIG. 3 corresponds to Table 1.3, which provides the microbiome features as described by bacterial taxa for detection of gut microbiome health in cats throughout progressive life stages. Tables 1.3 (i)-(xv) each correspond to a section of Table 1.3, which are joined together as shown in the figure to form the full Table 1.3.

FIG. 4 corresponds to Table 1.4, which provides the bacterial families associated with health in mammals and with utility for the detection of health in cats.

### DETAILED DESCRIPTION

### The Health of the Microbiome

The methods of the present disclosure can be used to determine the health of a feline's microbiome. This can be achieved by quantitating four or more bacterial species in a sample obtained from the feline to determine their abundance; and comparing the abundance to the abundance of the same species in a control data set. Changes in the abundance of the at least four bacterial species, compared to a control data set, suggest that the microbiome is less healthy and can be unhealthy. Following the determination, the owner can then seek veterinary intervention for the feline and the feline will likely benefit from an intervention to bring the microbiome back to its healthy state.

The presently disclosed subject matter has identified that bacterial species from certain bacterial taxa are indicative of a healthy microbiome. These taxa are shown in tables 1.1 and 1.3. In some embodiments, the bacterial species are genera selected from the group consisting of

[Eubacterium], [Eubacterium] hallii group, Anaerobiospirillum, Anaerostipes, Anaerotruncus, Bifidobacterium, Blautia, Butyricicoccus, Catenibacterium, Clostridium sensu stricto 1, Collinsella, Coriobacteriaceae, Faecalibacterium, Holdemanella, Lachnoclostridium, Lachnospiraceae, Lachnospiraceae [Eubacterium] hallii group, Lachnospiraceae [Ruminococcus] gauvreauii, Lachnospiraceae FCS020 group, Lachnospiraceae genus, Lachnospiraceae NK4A136 group, Lactobacillus, Megamonas, Megasphaera, Peptoclostridium [Clostridium], Romboutsia, Roseburia, Ruminococcaceae, Ruminococcaceae UCG-009, Sarcina, Sellimonas, Subdoligranulum, Succinivibrio, and Turicibacter.

In further embodiments, the bacterial species are selected from the group consisting of [Clostridium] hiranonis, [Eubacterium] brachy, [Eubacterium] hallii group sp., Anaerobiospirillum succiniciproducens, Anaerostipes sp., Anaerotruncus sp., Bifidobacterium sp., Bifidobacterium saeculare, Blautia [Ruminococcus] gnavus, Blautia sp., Butyricicoccus sp., Catenibacterium sp., Clostridium sensu stricto 1 sp., Collinsella sp., Coriobacteriaceae UCG-002/UCG-003, Faecalibacterium sp., Holdemanella sp., Lachnoclostridium sp., Lachnospiraceae sp., Lachnospiraceae [Eubacterium] hallii group sp., Lachnospiraceae [Ruminococcus] gauvreauii group sp., Lachnospiraceae [Ruminococcus] gauvreauii group sp., Lachnospiraceae FCS020 group sp., Lachnospiraceae genus sp., Lachnospiraceae NK4A136 group sp., Lactobacillus sp., Megamonas sp., Megasphaera sp., Romboutsia sp., Roseburia sp., Ruminococcaceae sp., Sarcina sp., Sellimonas sp., Subdoligranulum sp., Succinivibrio sp., Turicibacter sp., and UCG-009 sp.

Furthermore, in certain non-limiting embodiments, the bacterial species has a 16S rDNA sequence comprising a sequence having at least about 95%, at least about 96%, at least about 97%, at least about 98%, at least about 99% or 100% identity to the sequence of any one of the sequences selected from the group consisting of SEQ ID NOs: 3-73. In some embodiments, the bacterial species has a 16S rDNA sequence of any one of SEQ ID NO: 3-73.

In addition to those described herein, techniques which allow a skilled person to detect and quantitate bacterial taxa are well known in the art. These include, for example, polymerase chain reaction (PCR), quantitative (qPCR), 16S rDNA amplicon sequencing, shotgun sequencing, metagenome sequencing, Illumina sequencing, and nanopore sequencing. In some embodiments, the bacterial taxa are determined by sequencing or detection of the 16s rDNA sequence. In some embodiments, the bacterial taxa are determined by sequencing the V4-V6 region, for example using Illumina sequencing. These methods can use the primers 319F:

319F:
(SEQ ID NO: 1)
CAAGCAGAAGACGGCATACGAGATGTGACTGGAGTTCAGA
CGTGTGCTCTTCCGATCT
and
806R:
(SEQ ID NO: 2)
AATGATACGGCGACCACCGAGATCTACACTCTTTCCCTAC
ACGACGCTCTTCCGATCT.

The bacterial species can also be detected by other means known in the art such as, for example, RNA sequencing, protein sequence homology, qPCR or detection of other biological marker indicative of the bacterial species.

The sequencing data can be used to determine the presence or absence of different bacterial taxa in the sample. For example, the sequences can be clustered at about 98%, about 99% or 100% identity and abundant taxa (e.g. those representing more than 0.001 of the total sequences) can then be assessed for their relative proportions. Suitable techniques are known in the art and include, for example, logistic regression, partial least squares discriminate analysis (PLSDA) or random forest analysis and other multivariate methods.

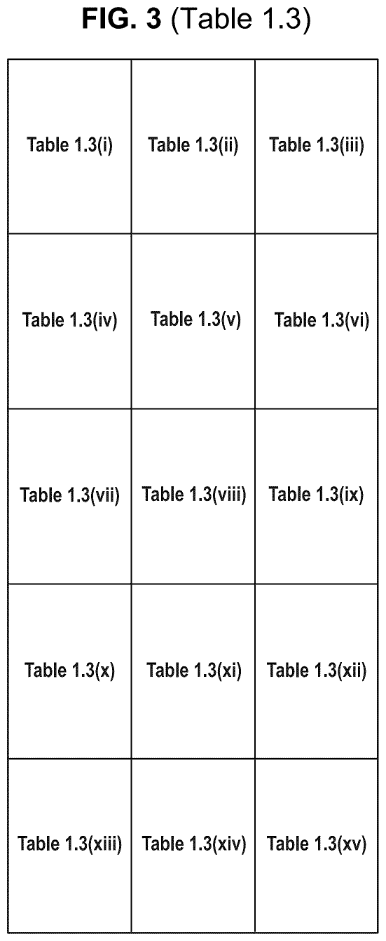

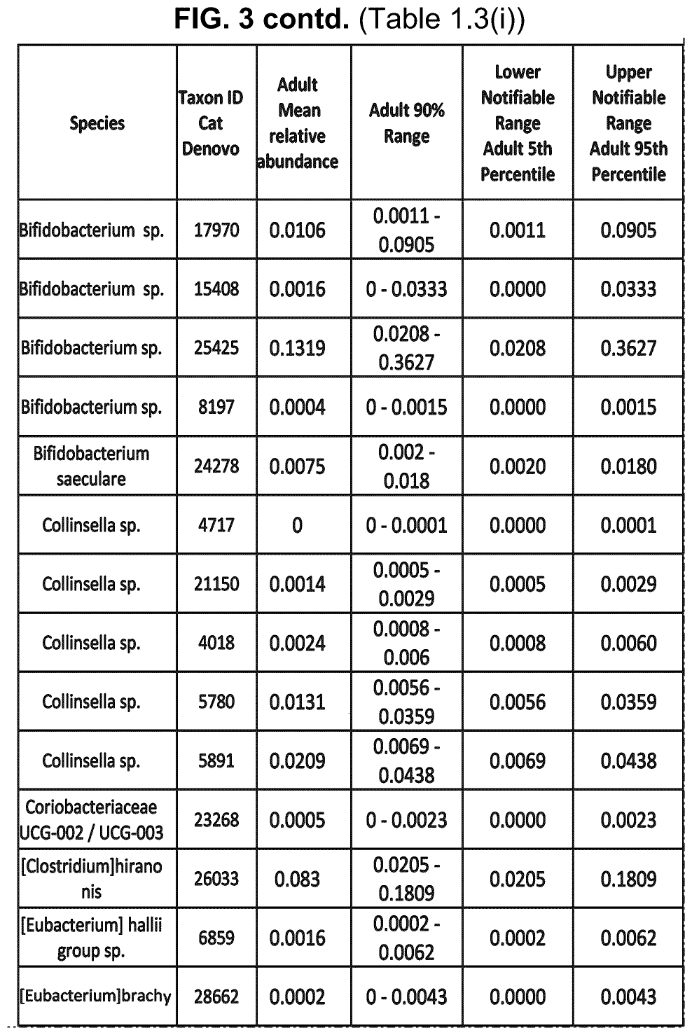

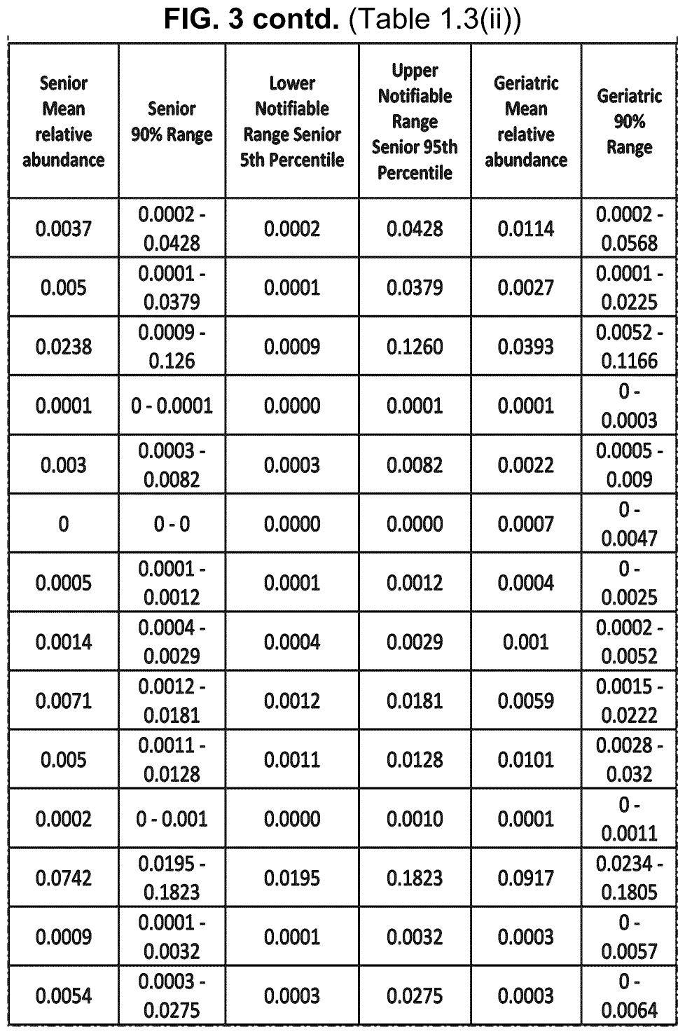

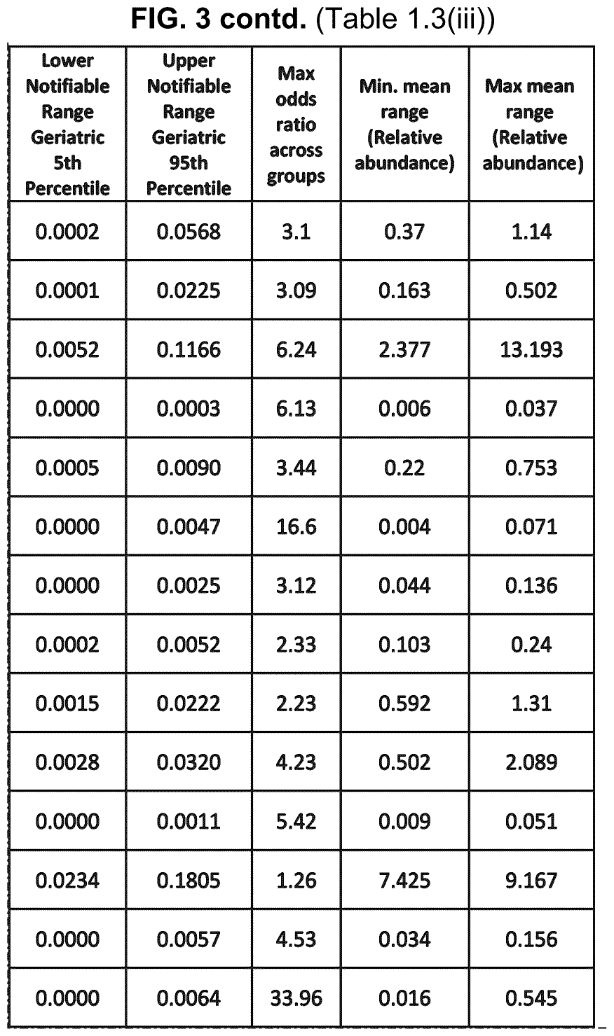

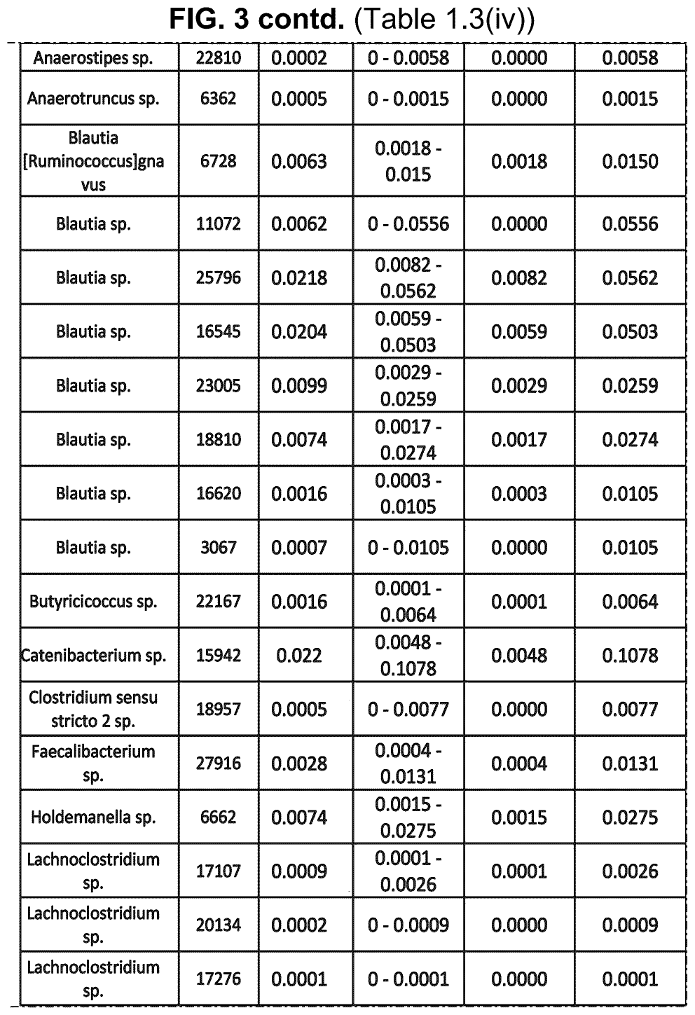

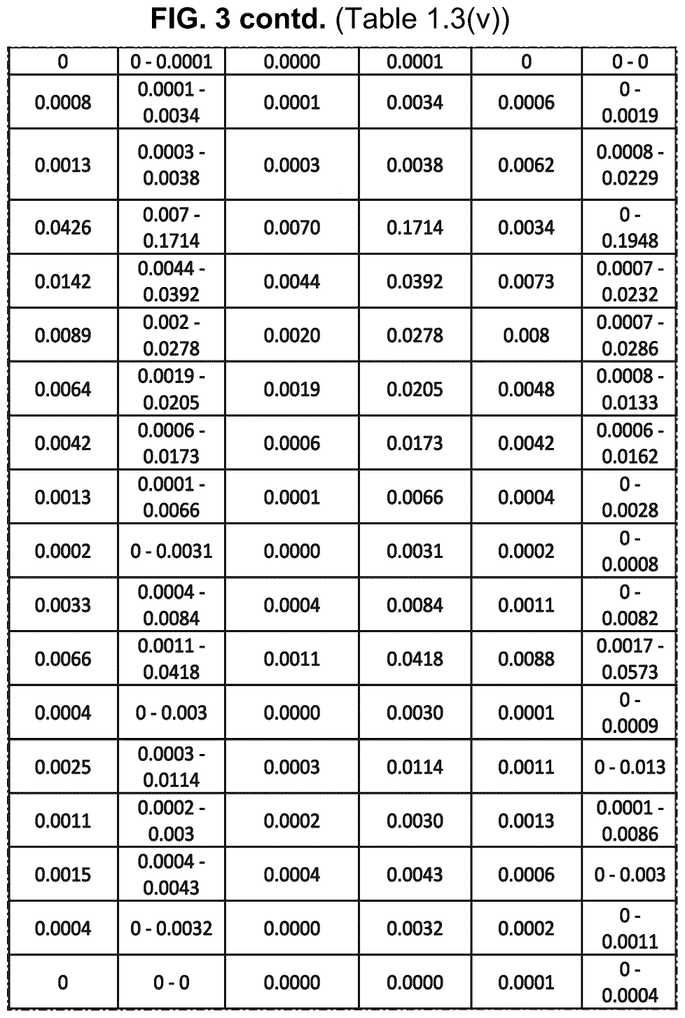

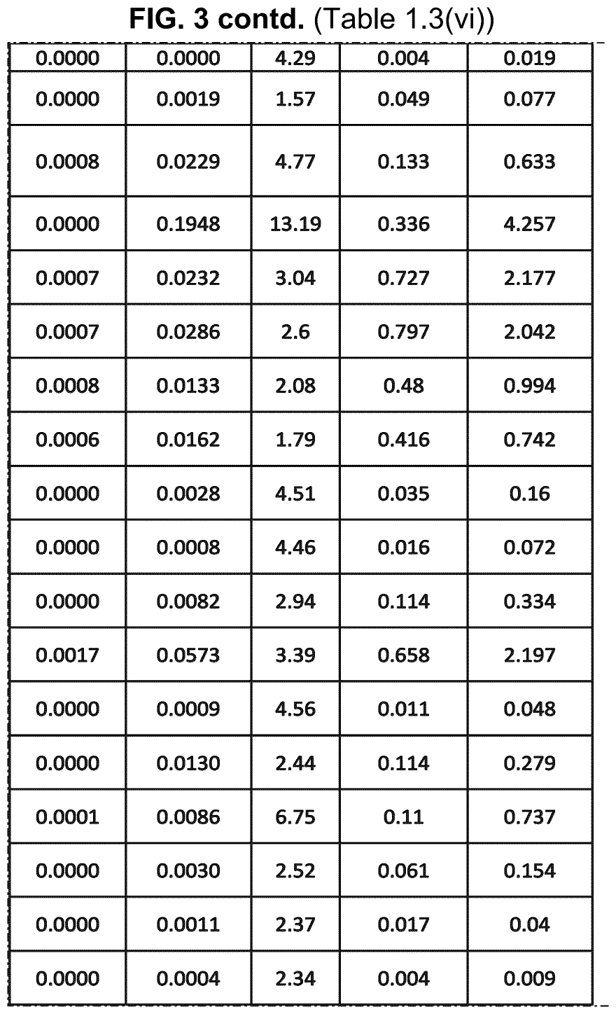

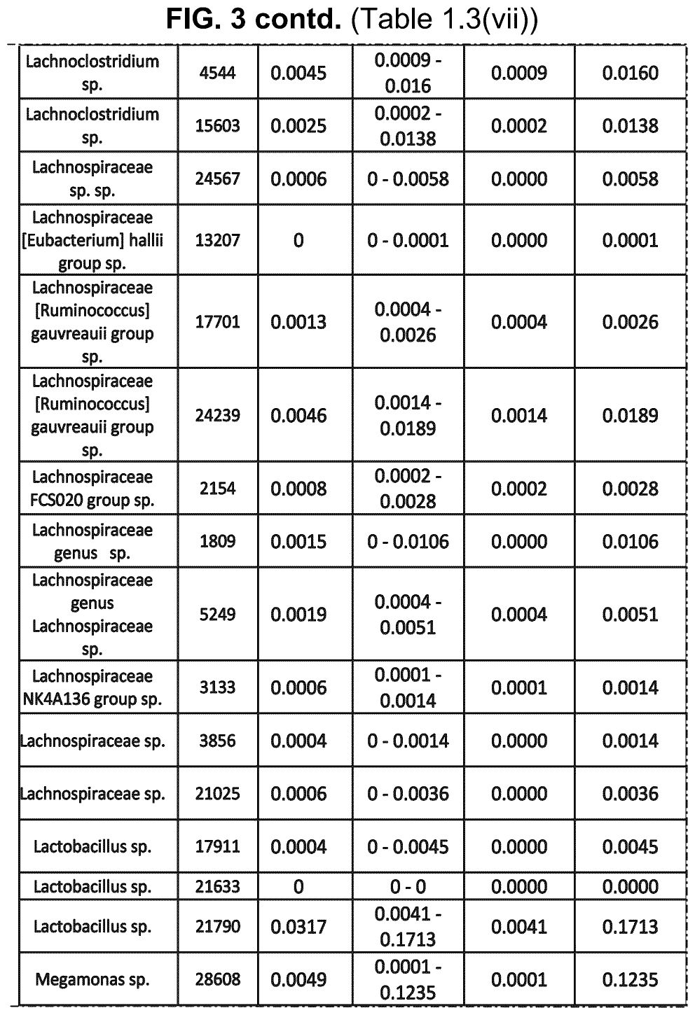

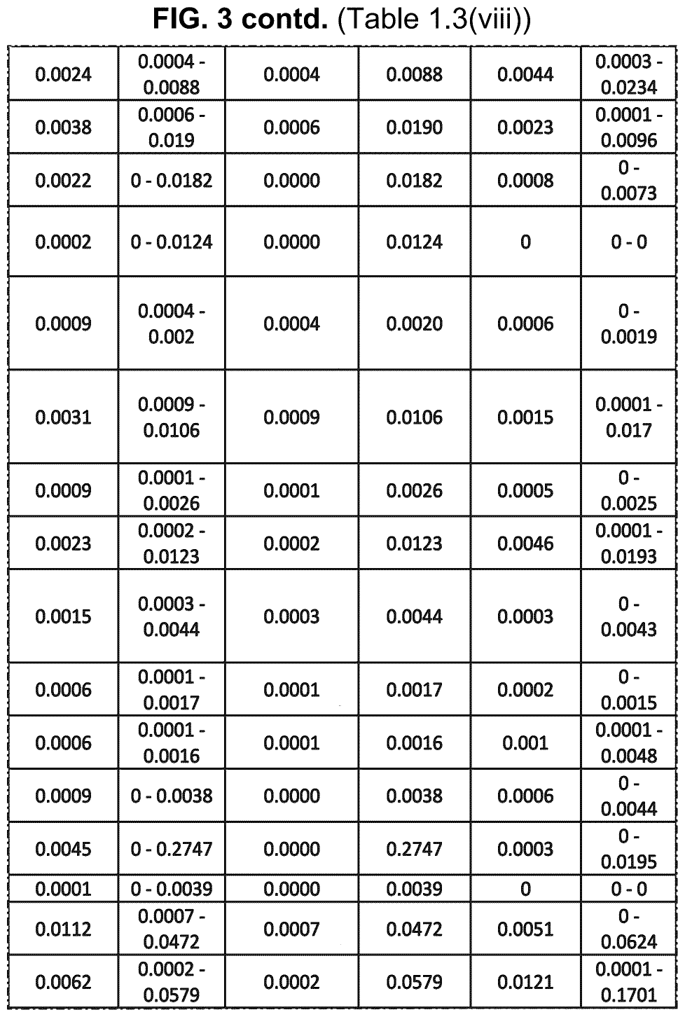

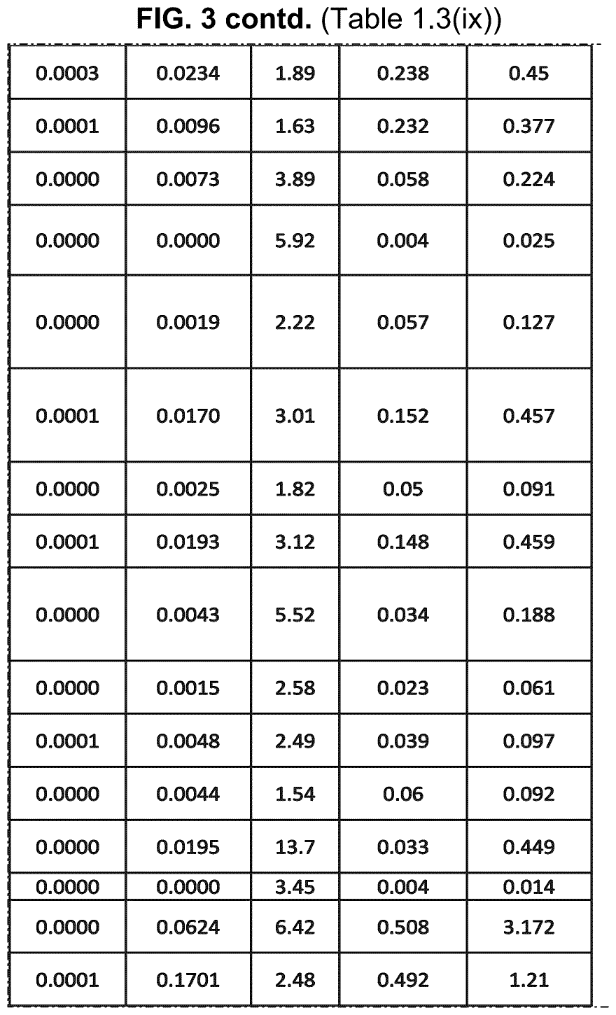

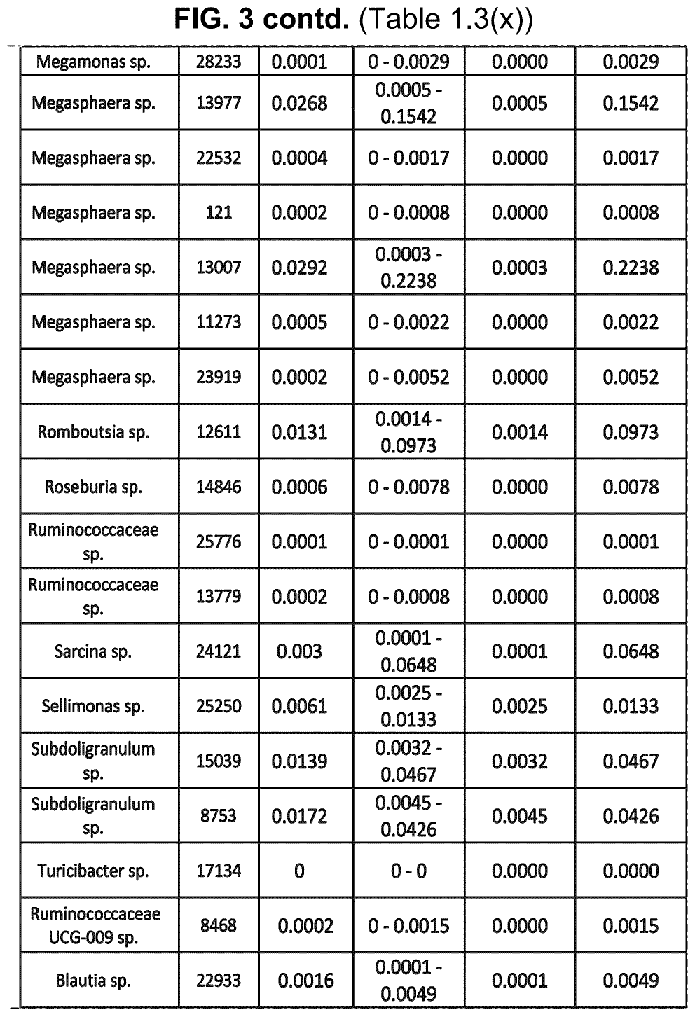

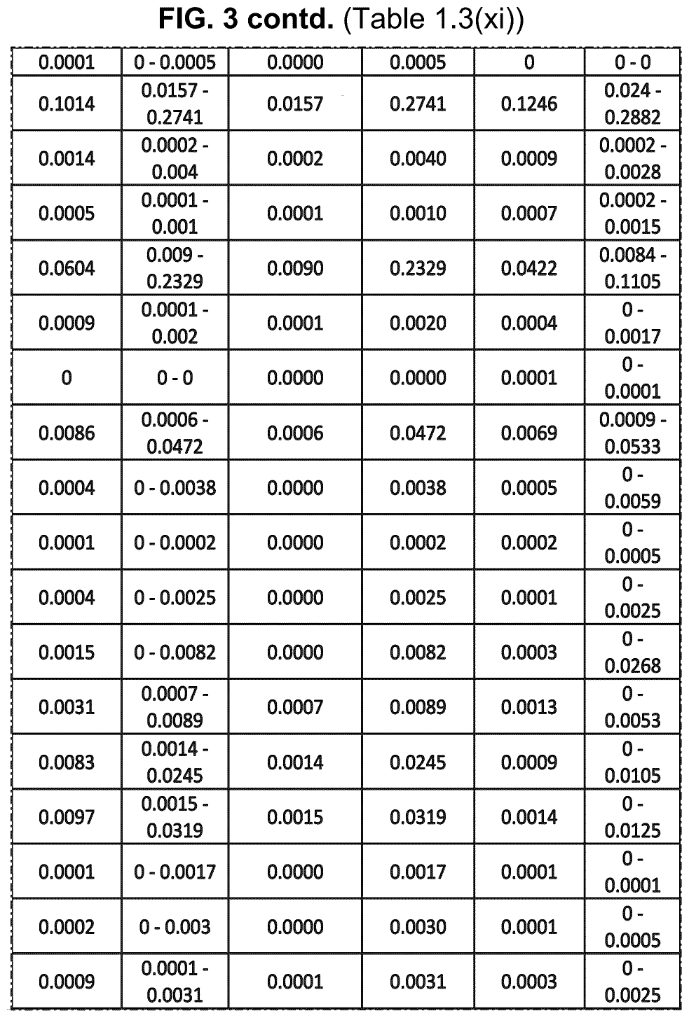

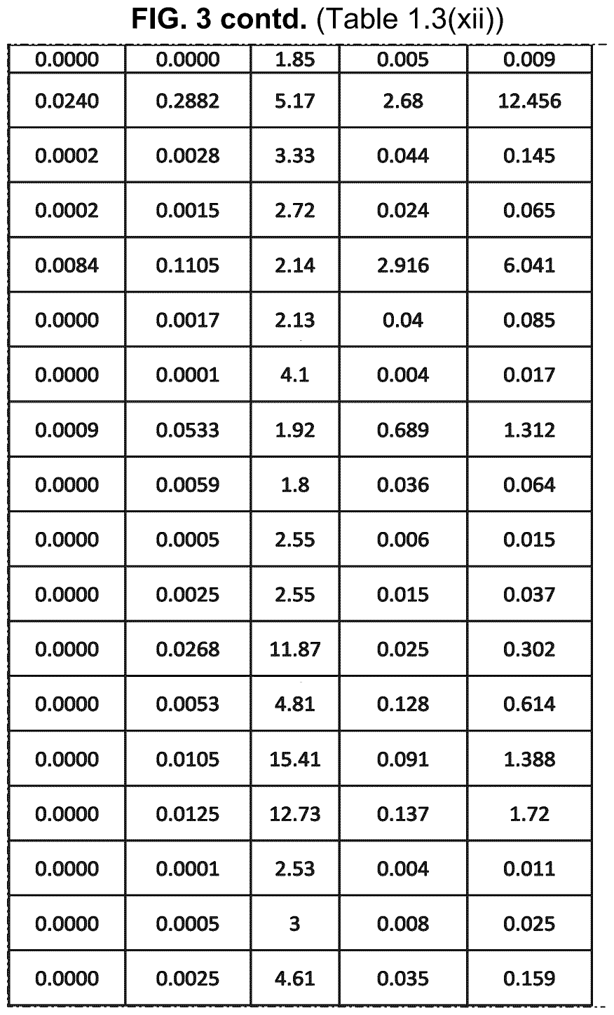

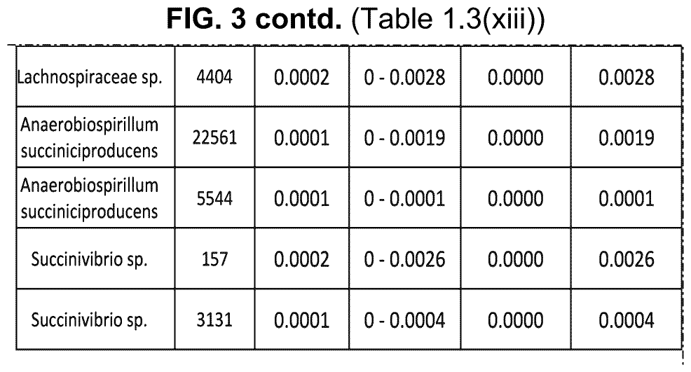

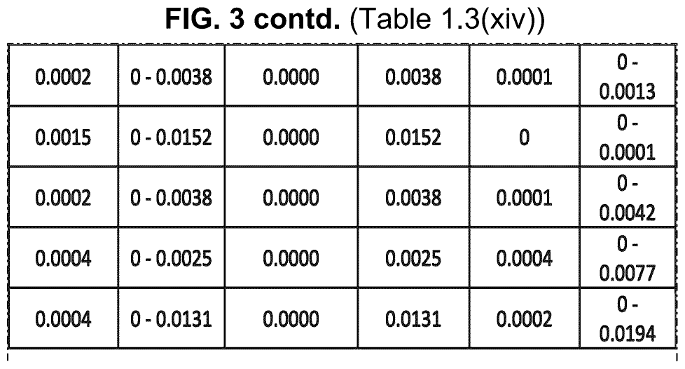

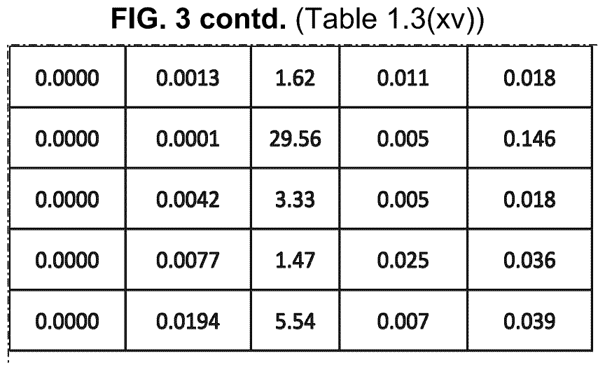

As will be apparent to a skilled person in the art, the abundance of these taxa in the microbiome will vary between different healthy individuals, but in a healthy population can generally be found within the range shown in FIG. 3 (Table 1.3). Thus, a bacterial taxa will be considered within a healthy range if it falls within the “90%” range shown in FIG. 3 (Table 1.3), taking into account the life stage of the feline. In such embodiments, the abundance of the bacterial taxa which is analysed will be compared to the “90%” value shown in FIG. 3 (Table 1.3) for the same bacterial taxa. For example, when bacteria of the genus Anaerostipes are analysed, the abundance of Anaerostipes will be deemed to be in a healthy range if the amount is within the range shown for Anaerostipes in FIG. 3 (Table 1.3), i.e. 0-0.0058. Thus, the abundance of bacterial genus or family can be increased or decreased relative to the abundance shown in FIG. 3 (Table 1.3). Furthermore, in some embodiments, when there are different ranges across a genus in FIG. 3 (Table 1.3), the ranges specific to a particular OTU is used in the methods disclosed herein, rather than using the values for the genus.

The healthy range for each bacterial species in a feline's microbiome can differ between different life stages. For example, the healthy range for Bifidobacterium sp. (Cat Denovo OTU_ID 17970) is between about 0.0011 and about 0.0905 in an adult feline, between about 0.0002 and about 0.0428 in a senior feline, and between about 0.0002 and about 0.0568 in a geriatric feline. Thus, a skilled person will understand that the healthy range needs to be determined with respect to the feline's life stage.

In addition, or alternatively, the feline's microbiome health can be assessed by determining the diversity of bacterial species within a feline's microbiome. To this end, the diversity index of the bacterial species within the feline's microbiome is determined and compared to the diversity index of a control data set. Diversity indices such as the Shannon diversity index or Simpson diversity or other measure of alpha or beta diversity can be used. In certain embodiments, the diversity index which is used is the Shannon diversity index.

For a healthy adult, the range in the mean diversity index is from about 3.14 to about 3.60; for a healthy senior feline, the healthy mean range is from about 2.93 to about 3.40; and for a geriatric feline, the healthy mean range is from about 2.51 to about 3.254. Where the microbiome diversity index falls outside this range, it is not always necessary to seek treatment. This will generally be useful, however, where the diversity index falls above or below a certain “intervention point”. These intervention points are listed in Table 1.0-A below:

TABLE 1.0-A

| | Lower | Upper |
|---|---|---|
| | intervention | intervention |
| Life stage | point | point |
| Adult | <2.77 | >3.99 |
| Senior | <2.41 | >3.92 |
| Geriatric | <2.28 | >4.17 |

In some embodiments, when the diversity index falls outside the range discussed above, the method can comprise a further step of changing the composition of the microbiome, as discussed below. This is particularly preferred where the diversity index falls above or below the notification point, as shown above.

### The Control Data Set

The abundance of the bacterial species is compared to a control data set from a feline from a similar life stage, e.g. a kitten, a juvenile feline, an adult feline, a senior feline, or a geriatric feline. FIG. 3 (Table 1.3) provides a suitable control data set against which the microbiome composition from the feline can be compared.

Alternatively, or in addition, a control data set can be prepared. To this end, the microbiome of two or more (e.g., 3, 4, 5, 10, 15, 20 or more) healthy felines can be analysed for the abundance of the species contained in the microbiome. A healthy feline in this context is a feline who has not been diagnosed with a disease that is known to affect the microbiome. Examples of such diseases include irritable bowel syndrome, ulcerative colitis, Crohn's and inflammatory bowel disease. The two or more felines will generally be from a particular life stage. For example, they can be kittens, juvenile felines, adult felines, senior felines or geriatric felines. This is useful because the microbiome changes in a feline's lifetime and the microbiome therefore needs to be compared to a feline at the same life stage. Where the feline is a cat, the control data set can further be from a cat of the same breed or, where the cat is a cross-breed, the same breed as one of the direct ancestors (parents or grandparents) of the cat.

Specific steps to prepare the control data set can comprise analysing the microbiome composition of at least two (e.g., 3, 4, 5, 6, 7, 8, 9, 10, 15, 20 or more) kittens, and/or at least two (e.g., 3, 4, 5, 6, 7, 8, 9, 10, 15, 20 or more) juvenile felines, and/or at least two (e.g., 3, 4, 5, 6, 7, 8, 9, 10, 15, 20 or more) adult felines, and/or at least two (e.g., 3, 4, 5, 6, 7, 8, 9, 10, 15, 20 or more) senior felines and/or at least two (e.g., 3, 4, 5, 6, 7, 8, 9, 10, 15, 20 or more) geriatric felines; determining the abundance of a bacterial species (e.g., the bacterial species discussed above); and compiling these data into a control data set.

For embodiments where the diversity index of the microbiome is determined, the control data set can be prepared in a similar manner. In particular, the diversity index can be determined in two or more (e.g., 3, 4, 5, 6, 7, 8, 9, 10, 15, 20 or more) healthy felines at a particular life stage (kitten, juvenile, adult, senior or geriatric). The results can then be used to identify the mean range for the diversity index in a feline at that life stage.

It will be understood that the control data set does not need to be prepared every time the method of the present disclosure is performed. Instead, a skilled person in the art can rely on an established control set.

The feline

The methods of the present disclosure can be used to determine the microbiome health of a feline. This genus comprises species in the Felidae family. These species include African-Asian Wildcat (Felis silvestris ornata), African Golden Cat (Profelis aurata), Andean Mountain Cat (Leopardus jacobita), Asiatic Golden Cat (Catopuma temminckii), Bay Cat (Catopuma badia), Black-footed Cat (Felis nigripes), Bobcat (Lynx rufus), Bornean Clouded Leopard (Neofelis diardi), Canadian Lynx (Lynx canadensis), Caracal (Caracal caracal), Cheetah (Acinonyx jubatus), Chinese Desert Cat (Felis bieti), Clouded Leopard (Neofelis nebulosa), Domestic Cat (Felis catus), Eurasian Lynx (Lynx lynx), European Wildcat (Felis silvestris), Fishing Cat (Prionailurus viverrinus), Flat-headed Cat (Prionailurus planiceps), Geoffroy's Cat (Leopardus geoffroyi), Iberian Lynx (Lynx pardinus), Iriomote Cat (Prionailurus iriomotensis), Jaguar (Panthera onca), Jaguarundi (Herpailurus yagouarundi), Jungle Cat (Felis chaus), Kodkod (Leopardus guigna), Leopard Cat (Prionailurus bengalensis), Leopard (Panthera pardus), Lion (Panthera leo), Marbled Cat (Pardofelis marmorata), Margay (Leopardus wiedii), Mountain Lion (Puma concolor), Ocelot (Leopardus pardalis), Oncilla (Leopardus tigrinus), Pallas's Cat (Otocolobus manul), Pampas Cat (Leopardus colocolo), Rusty-spotted Cat (Prionailurus rubiginosus), Sand Cat (Felis margarita), Serval (Leptailurus serval), Snow Leopard (Uncia uncia), and Tiger (Panthera). In some embodiments, the feline is a domestic cat, herein referred to as a cat.

Furthermore, in some embodiments, the feline is healthy. “Healthy,” as used herein, refers to a feline that has not been diagnosed with a disease that is known to affect the microbiome. Examples of such diseases include, but are not limited to, irritable bowel syndrome, ulcerative colitis, Crohn's and inflammatory bowel disease. In certain embodiments, the feline does not suffer from dysbiosis. Dysbiosis refers to a microbiome imbalance inside the body, resulting from an insufficient level of keystone bacteria (e.g., bifidobacteria, such as B. longum subsp. infantis) or an overabundance of harmful bacteria in the gut. Methods for detecting dysbiosis are well known in the art.

One advantage of the methods disclosed herein is that they allow a skilled person to determine whether the feline's microbiome is healthy, taking into account the feline's life stage.

There are numerous different breeds of cats. A summary of the different life stages is provided in the Table 1.0-B below.

TABLE 1.0-B

| Youth | Adult | Senior | Geriatric |
|---|---|---|---|
| 1 to 4 years | 5 to 9 years | 10 to 13 years | ≥14 years |

A skilled person will appreciate that the age ranges discussed above will not always strictly apply to each individual feline. Rather, a skilled person will be able to categorise a feline into a specific life stage by its physiological features, for example.

### The Sample

According to the methods disclosed herein, the sample from which the bacterial species are analysed can be, in some embodiments, a faecal sample or a sample taken from the gastrointestinal lumen of the feline. Faecal samples are convenient because their collection is non-invasive and it also allows for easy repeated sampling of individuals over a period of time. However, other samples can also be used in the methods disclosed herein, including, but not limited to, ileal, jejunal, duodenal samples and colonic samples.

In some embodiments, the sample is a fresh sample. In other embodiments, the sample is frozen or stabilised by other means, such as addition to preservation buffers, or by dehydration using methods such as freeze drying, before use in the methods of the present disclosure.

Before use in the disclosed methods, in some embodiments, the sample is processed to extract DNA. Methods for isolating DNA are well known in the art, as reviewed in reference [20], for example. These include, for example, the Qiagen DNeasy kit™, the MoBio PowerFecal kit™, Qiagen QIAamp Cador Pathogen Mini kit™, the Qiagen QIAamp DNA Stool Mini Kit™ as well as Isopropanol DNA Extraction. A further useful tool to use with the methods of the present disclosure is the QIAamp Power Faecal DNA kit (Qiagen). Other ways of isolating DNA that are known in the art can also be used in the methods disclosed herein.

### Changing the Microbiome

In some embodiments, the methods of the present disclosure comprises a further step of changing the composition of the microbiome. The composition of the microbiome can be changed by administering to the feline a dietary change, a functional food, a supplement, or a nutraceutical or pharmaceutical composition that is capable of changing the composition of the microbiome. Such functional foods, nutraceuticals, live biotherapeutic products (LBPs), and pharmaceutical compositions are well known in the art and can comprise bacteria [21]. They can comprise single bacterial species selected from the group consisting of Bifidobacterium sp. such as B. animalis (e.g., B. animalis subsp. animalis or B. animalis subsp. lactis), B. bifidum, B. breve, B. longum (e.g., B. longum subsp. infantis or B. longum subsp. longum), B. pseudolongum, B. adolescentis, B. catenulatum, or B. pseudocatanulatum; single bacterial species of Lactobacillus, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius, L. paracasei, L. kisonensis, L. paralimentarius, L. perolens, L. apis, L. ghanensis, L. dextrinicus, L. shenzenensis, L. harbinensis; or single bacterial species of Pediococcus, such as P. parvulus, P. lolii, P. acidilactici, P. argentinicus, P. claussenii, P. pentosaceus, or P. stilesii or similarly species of Enterococcus such as E. faecium; or Bacillus species such as Bacillus subtilis, B. coagulans, B. indicus, or B. clausii. In alternative embodiments, the methods can include combinations of these and other bacterial species. The amount of the dietary change, the functional food, the supplement, the nutraceutical composition, or the pharmaceutical composition that is administered to the feline can be an amount that is effective to effect a change in the composition of the microbiome.

The further step of changing the composition of the microbiome can be performed in instances where a feline's biological microbiome is found to be unhealthy. In that case, it can be highly desirable to make a dietary change and/or to administer a nutraceutical or pharmaceutical composition to shift the microbiome back to a healthy state, as determined by a method of the present disclosure.

The methods of the present disclosure can also be used to assess the success of a treatment as described above. To this end, a feline can undergo a dietary change and/or receive a nutraceutical or pharmaceutical composition which is capable of changing the composition of the microbiome. Following commencement of the treatment (e.g., administration of the pharmaceutical composition), for example after about 1 day, 2 days, 5 days, 1 week, 2 weeks, 3 weeks 1 month, etc., the health of the microbiome can be assessed using any of the methods of the present disclosure. In certain embodiments, the health of the microbiome is determined before and after administration of the pharmaceutical or nutraceutical composition.

### Monitoring

In some embodiments, the methods described herein are performed once to determine a feline's microbiome health. In other embodiments, the methods described herein are performed more than once, for example two times, three times, four times, five times, six times, seven times, or more than seven times. This allows the health of the microbiome to be monitored over time. This can be useful for example where a feline is receiving treatment to shift the microbiome. The first time the method is performed, the health of the microbiome is determined and, following a dietary change or administration of a functional food, nutraceutical or pharmaceutical composition, the method is repeated to assess the influence of the treatment on the health of the microbiome. The health of the microbiome can also be determined for the first time after the feline has received treatment and the method repeated afterwards to assess whether there is a change in the health of the microbiome.

The methods described herein can be repeated about one week, about two weeks, about three weeks, about one month, about two months, about three months, about four months, about five months, about six months, about 12 months, about 18 months, about 24 months, about 30 months, about 36 months, or more than about 36 months apart.

### Treatment

In some embodiments, the methods of the present disclosure can also relate to methods for treating a feline having an unhealthy microbiome. In some embodiments, the methods for treating include (i) identifying the feline as requiring treatment by determining the unhealthy status of the microbiome according to any of the methods disclosed herein, and (ii) administering to the feline a dietary change, a functional food, a supplement, a nutraceutical, or a pharmaceutical composition as disclosed herein that is capable of changing the composition of the microbiome. The amount of the dietary change, the functional food, the supplement, the nutraceutical composition, or the pharmaceutical composition that is administered to the feline can be an amount that is effective to effect a change in the composition of the microbiome, or to improve any symptoms relating to the feline having an unhealthy microbiome status. Optionally, in some embodiments, the method further includes determining the microbiome health of the feline following the administration of the dietary change, the functional food, the supplement, the nutraceutical, or the pharmaceutical composition to evaluate the effectiveness of the treatment.

### General

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

The practice of the present disclosure will employ, unless otherwise indicated, conventional methods of chemistry, biochemistry, molecular biology, immunology and pharmacology, within the skill of the art. Such techniques are explained fully in the literature. See, e.g., references [22-29], etc.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref [30]. A preferred alignment is determined using the BLAST (basic local alignment search tool) algorithm or Smith-Waterman homology search algorithm using an affine gap search with a gap open penalty of 12 and a gap extension penalty of 2, BLOSUM matrix of 62. The Smith-Waterman homology search algorithm is disclosed in ref. [31]. The alignment can be over the entire reference sequence, i.e. it can be over 100% length of the sequences disclosed herein.

### Definitions

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “containing,” and “comprising” are interchangeable and one of skill in the art is cognizant that these terms are open ended terms. Further, the term “comprising” encompasses “including” as well as “consisting,” e.g., a composition “comprising” X can consist exclusively of X or can include something additional, e.g., X+Y.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, alternatively up to 10%, alternatively up to 5%, and alternatively still up to 1% of a given value. Alternatively, particularly with respect to biological systems or processes, the term can mean within an order of magnitude, preferably within 5-fold, and more preferably within 2-fold, of a value. In certain embodiments, the term “about” in relation to a numerical value x is optional and means, for example, x±10%.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition (e.g., a dietary change, a functional food, a supplement, a nutraceutical composition, or a pharmaceutical composition) to change the composition of a microbiome in a feline having an unhealthy microbiome, the effective amount is an amount sufficient to bring the health status of the microbiome back to a healthy state, which is determined according to one of the methods disclosed herein. In certain embodiments, an effective treatment as described herein can also include administering a treatment in an amount sufficient to decrease any symptoms associated with an unhealthy microbiome. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of an unhealthy microbiome. An effective amount can be administered in one or more administrations. A likelihood of an effective treatment described herein is a probability of a treatment being effective, i.e., sufficient to alter the microbiome, or treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. In certain embodiments, the decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

The word “substantially” does not exclude “completely” e.g. a composition which is “substantially free” from Y can be completely free from Y. Where necessary, the word “substantially” can be omitted from the definition of the present disclosure.

Unless specifically stated, a process or method comprising numerous steps can comprise additional steps at the beginning or end of the method, or can comprise additional intervening steps. Also, steps can be combined, omitted or performed in an alternative order, if appropriate.

Various embodiments of the methods of the present disclosure are described herein. It will be appreciated that the features specified in each embodiment can be combined with other specified features, to provide further embodiments. In particular, embodiments highlighted herein as being suitable, typical or preferred can be combined with each other (except when they are mutually exclusive).

### EXAMPLES

The presently disclosed subject matter will be better understood by reference to the following Example, which is provided as exemplary of the invention, and not by way of limitation.

### Example 1. Species for Measurement of the Health Characteristics of the Feline Gut Microbiota and Microbiome

The objective of the study was to describe the gastrointestinal microbiota of healthy cats in adulthood including throughout the adult, senior and geriatric life stages. The primary endpoints of interest for the analysis were microbial diversity and community composition as measured by relative taxon abundance at species level (98% 16S rDNA sequence identity) across life stage groups in the context of the bacterial taxa associated with health in other mammalian hosts.

### Summary

The faecal microbiota, as an indicator of the gut microbiome, was assessed in a cohort of 48 cats in adult (aged 4.7-6.8 years), senior (aged 8.1-12.5 years) and geriatric (aged 12.6-16.2 years) life stages. The microbiota was described as highly complex with 113 abundant bacterial taxa (>0.05%) at the 98% species level and with numerically higher diversity observed in cats compared to in a similar study in dogs (feline mean diversity 3.09, 95% CI 2.47-3.70, 5th percentile 2.28, 95th percentile 4.03.; canine mean 2.70 95% CI 2.30-3.10, 5th percentile 1.74, 95th percentile 3.58). Altered relative abundance was observed in more than 20% (25 of 113) of the abundant bacterial taxa with life stage group even after 21 days of consistency in dietary intake. These differences in a range of bacterial taxa with diverse nutritional characteristics were observed in the microbiota of cats in different life stages, suggestive that changes can occur in the feline microbiome with age.

These insights provide signatures of the gastrointestinal microbiota in the healthy aging feline microbiome and, through comparison with the health associated microbiota in other monogastric mammals, microbiome characteristics indicative of health are described. These putatively health-associated microbiome characteristics can be leveraged in the development of health monitoring tools for tracking the health of the feline gut microbiome throughout life and enabling deployment of interventions to maintain health where required.

### Methods

A cross-sectional analysis of the faecal microbiota was conducted in a cohort of 48 cats aged between 4.7 and 16.2 years at the Mars Inc. Pet Health and Nutrition Centre (PHNC, Lewisburg, Ohio). Animals were assigned to one of 3 groups defined as different life stages including adult (target age range 3-6 years), senior (target age range 9.5-12 years) and geriatric (target age range 14+years) cats. Group assignment was based on age with specific categories guided by the findings of the research on evidence-based analysis of Banfield veterinary diagnoses with age in cats and dogs (Salt and Saito, personal communication). All cats were fed a consistent diet for a period of 30 days with fresh faecal samples collected at days 21, 24 and 28 (+/−2 days).

### Animals

The cohort comprised 20 adult cats (aged 4.7-6.8 years), 20 senior cats (aged 8.1-12.5 years) and 8 geriatric (aged 12.6-16.2 years) were recruited to the study. All animals received a veterinary health check to determine suitability for inclusion prior to the start of the study. Cats were provided with access to fresh drinking water at all times and were exercised consistently throughout the study as per standard practice for PHNC. All cats were involved in their normal daily activities throughout the study and received their standard medication as required. Cats were familiarised to study personnel and were socialised for a minimum of 1 hour each day following the standard PHNC care package.

### Diet

The cats were fed the same commercially available nutritionally complete diet (Royal Canin indoor 7+dry cat food) for a period of 30 days. Additionally, a 10 g bolus of RC wet cat food was fed daily across the cohort to facilitate feeding of medication in those cats with active veterinary prescriptions. Cats were fed at energy levels (mean energy requirements; MER) to maintain a healthy body condition score (BCS) and bodyweight (within +/−5%) throughout the study. Two equal food portions were offered (˜50% MER) twice a day.

### Data Collection

During the study the following co-variates were collected for inclusion in data analyses to establish whether differences in the microbiome were associated with adult, senior and geriatric life stages. Animal housing details; Daily food intake; Bodyweight and body condition score and daily and overnight faeces scores per room were recorded. Faeces were scored using the WALTHAM 17-point faeces quality scale and incidences of poor faeces (outside of the acceptable range 1.5-3.75) were recorded.

### Faeces Sample Collection, Processing and Analysis

Fresh faecal samples were collected no more than 15 minutes after defecation. Following collection, faeces were portioned into aliquots and stored at-80 degrees centigrade prior to processing for DNA extraction using the QIAamp Power Faecal DNA kit (Qiagen). DNA concentrations achieved per sample were determined by standard nanodrop DNA quantification methods. PCR amplification was conducted on individual samples to generate dual indexed, barcoded 16SrDNA sequencing libraries representing the V4-V6 region using DNA oligonucleotide primers 319F: CAAGCAGAAG ACGGCATACG AGATGTGACT GGAGTTCAGA CGTGTGCTCT TCCGATCT (SEQ ID NO: 1) and 806R: AATGATACGG CGACCACCGA GATCTACACT CTTTCCCTAC ACGACGCTCT TCCGATCT (SEQ ID NO: 2) suitable for analysis on the the Ilumina MiSeq sequencing system. DNA sequencing was conducted by Eurofins Applied Genomics Laboratory (Eurofins Genomics; Anzinger Str. 7a; 85560 Ebersberg; Germany. Samples were quantified and pooled prior to loading, library pool concentrations were determined prior to processing to optimise Ilumina channel loading. Quality thresholds of a minimum of 1,000 sequence reads per sample were defined for sequence data, which was de-noised and clustered based on percentage identity (98.5%) using the WALTHAM bioinformatics analysis pipeline. The resulting operational taxonomic unit (OTU) data for abundant taxa (representing >0.001 of the total sequences) were then assessed for their relative proportions and to determine whether the detection (presence/absence) or relative abundance of taxa were observed to contrast between groups.

### Statistical Methods

Prior to individual modelling of the bacterial OTUs which approximately represented individual species, rare OTUs were identified as those with a mean proportion of less than 0.05% and present in two or fewer samples from a single age group. After identification, rare OTUs were combined to create a single group. The relative abundance compared to the sample total for each clustered OTU, and for the combined rare group, was described and group means and ranges were calculated per OTU to describe the distribution of the OTU detection levels throughout the cohort. Mean range was defined as that between the upper and lower 95% CI and the 5th and 95th percentiles of the cohort range were calculated to inform on the outlying values per OTU.

Relative abundance data was analysed for assessment of whether contrasts existed individually using a generalised linear mixed effects model (GLMM) with a binomial distribution and logit link function. In the model, counts and total counts represented the response variables including life stage group as a fixed effect, with a random intercept of dog to account for the repeated measurements. All pairwise comparisons were performed between life stage groups using a permutation test permuting the group indicator for each pet. A familywise error rate of 5% was maintained using multiple comparisons correction. The associated primary measures were analysed with linear and generalised linear models, with random effects in the cases where repeated measures were taken per pet.

Shannon diversity was calculated for each sample and modelled using a linear mixed effects model with a fixed effect of age group and random intercept of pet. Pairwise comparisons of the life stage groups were performed with a controlled familywise error rate of 5%.

Exploratory analyses were performed using principal components analysis (PCA) and t-distributed stochastic neighbour embedding (t-SNE) to reduce the dimension of the data and visually represent groups based on taxon abundance data.

### Results

The cohort of 48 cats comprised 20 adult cats (mean age 5.66 years; 8 male; 12 female), 20 senior cats (mean age 10.10 years; 10 male; 10 female), and 8 geriatric cats (mean age 14.78 years; 3 male; 5 female).

High throughput sequence reads were sorted according to individual sequence tags resulting in, on average 49,485 (range 19,704-112,958) sequence reads per sample. Clustering of DNA sequences representative of bacterial taxa at 98% identity resulted in the identification of 29,295 species level OTUs. This total was reduced to 113 species level OTUs after removal of the rare OTUs to a pseudo group of ‘rare taxa’. Individual analysis of rare OTUs was not conducted since these taxa represented less than 0.05% of the sequences in less than two individuals from any single group.

Interrogation of the Silva database using 16SrDNA partial sequences facilitated taxonomic designations of bacterial species (OTUs) detected in faeces from adult senior and geriatric cats revealed microbial taxa associated with health in humans, other mammals, and/or in cats based on observed contrasts with life stage in the study data (Tables 2.1 and 2.2). Out of a total of 113 abundant taxa representing individual species, 61 (43%) were identified as bacterial species associated with health in non-canid mammals.

Differences in a range of bacterial taxa with diverse nutritional characteristics were observed in the microbiota of cats in different life stages, suggestive that changes occur in the feline microbiome with age. Significant contrasts in the relative abundance was observed between life stage groups for more than 20% (25 of 113) of the abundant bacterial taxa even after 21 days of consistency in dietary intake.

These insights provide putative signatures of an aging feline microbiome that can be leveraged in the development of microbiome health diagnostics and monitoring tools to support the detection of a healthy microbiome and facilitate the application of dietary interventions towards maintaining a healthy microbiome throughout life.

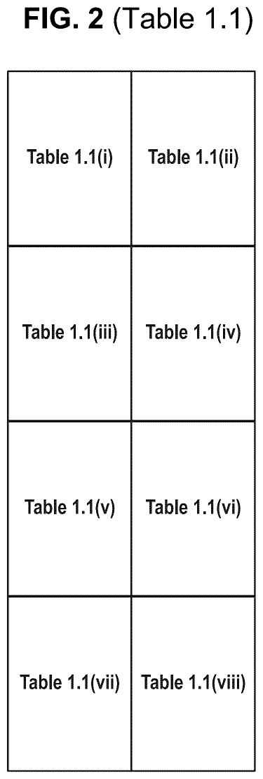

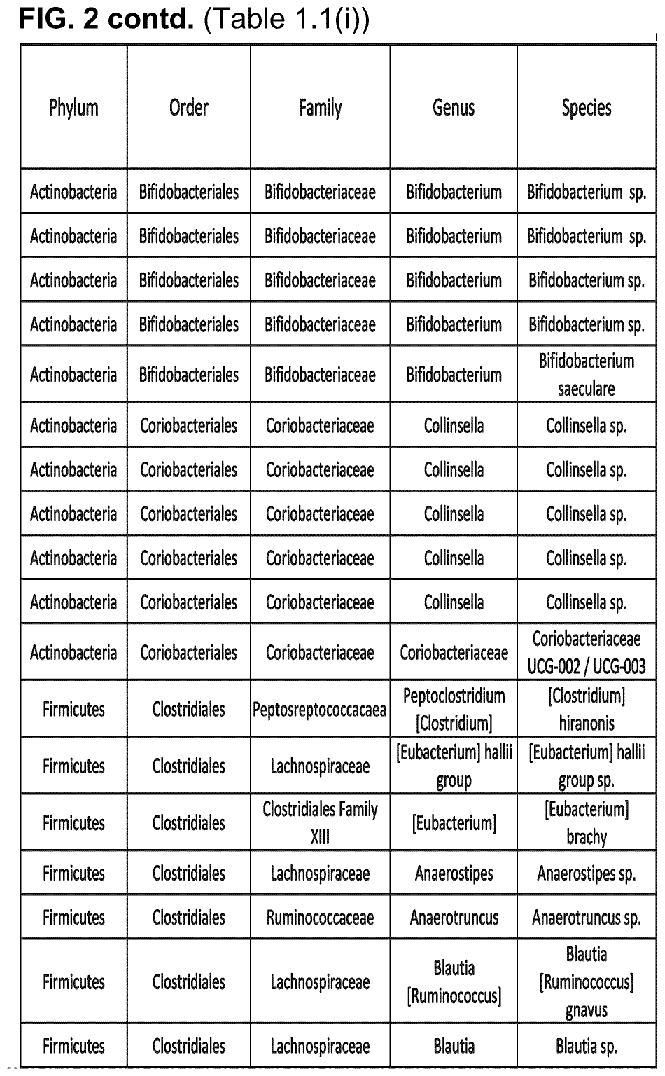

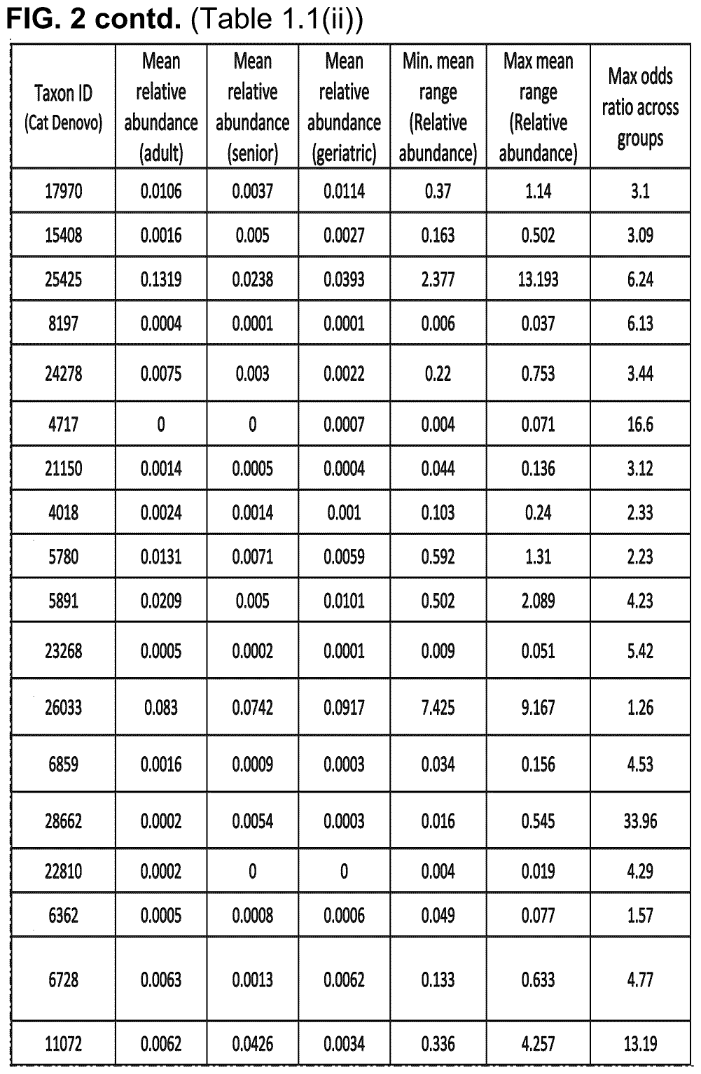

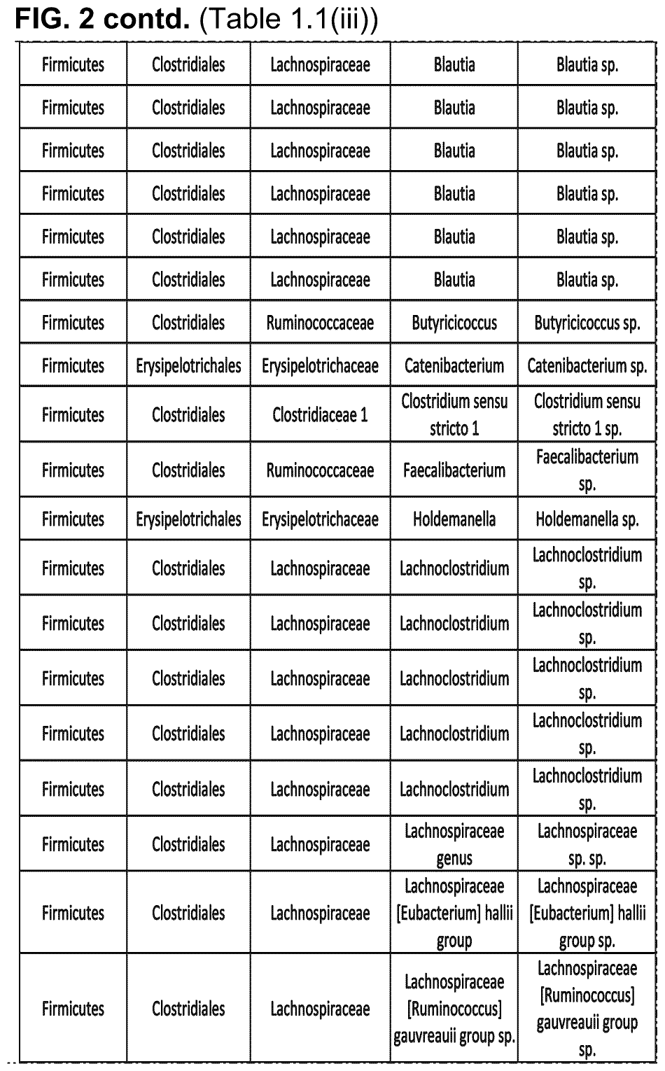

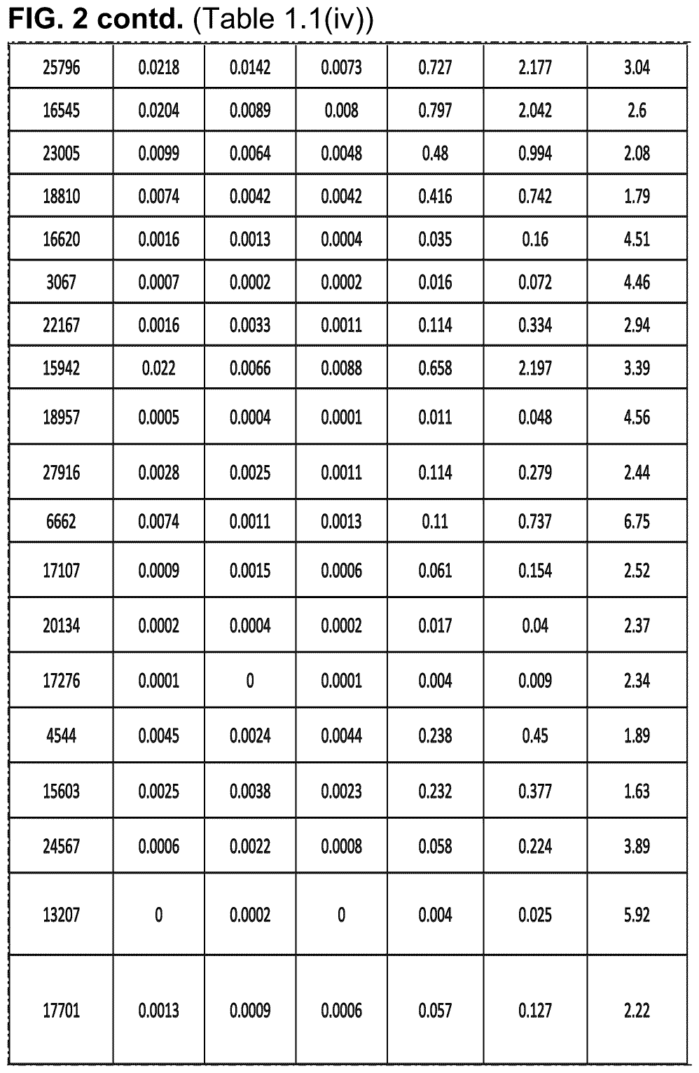

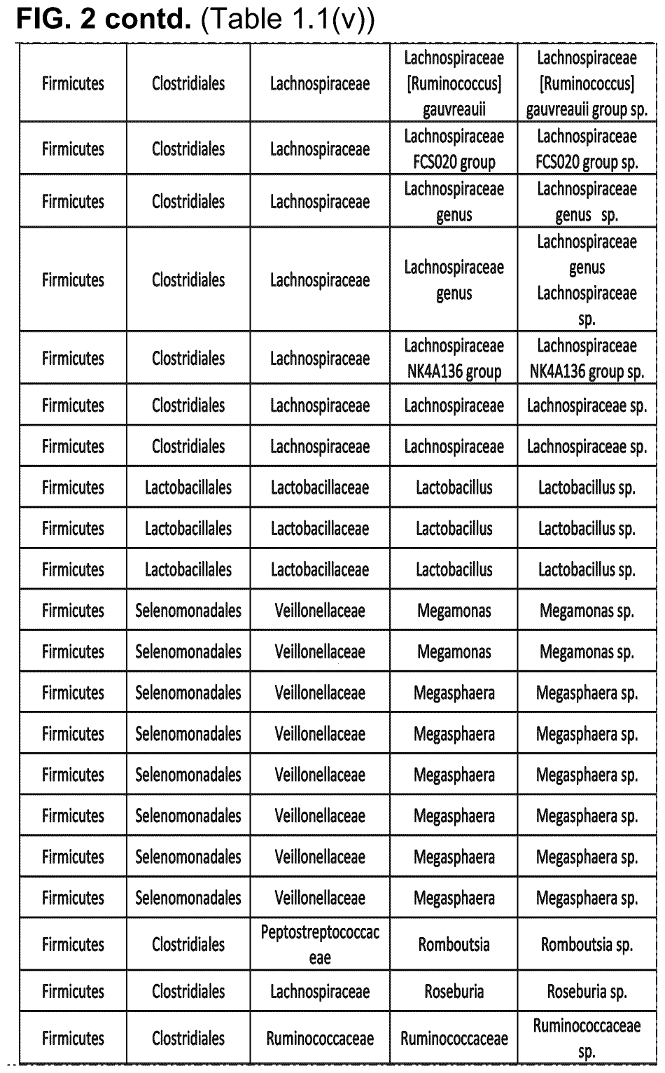

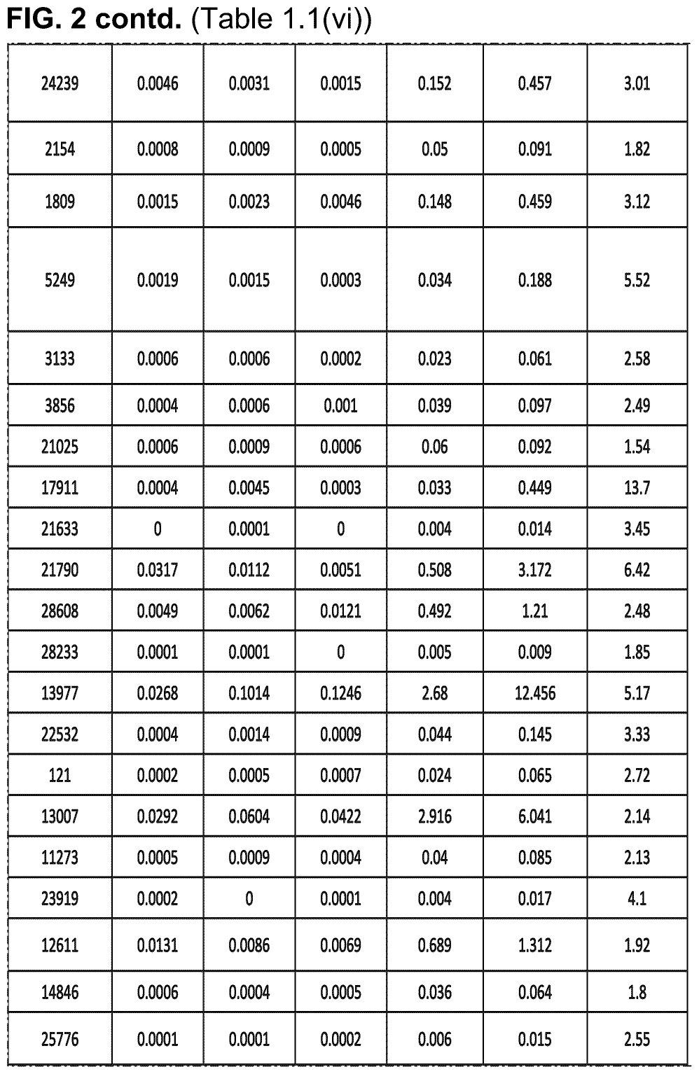

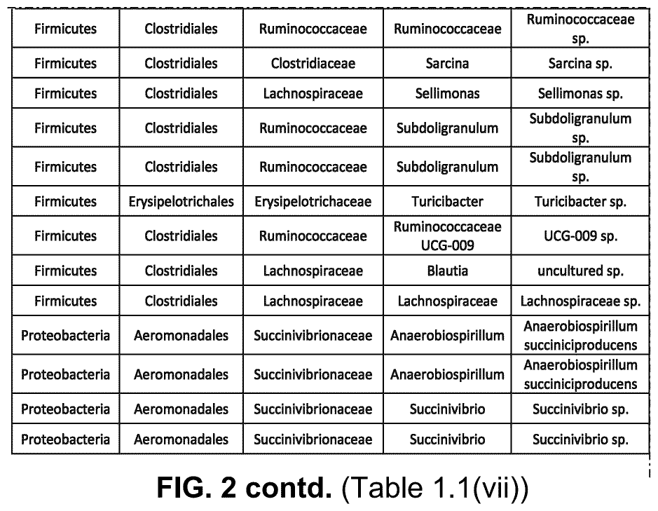

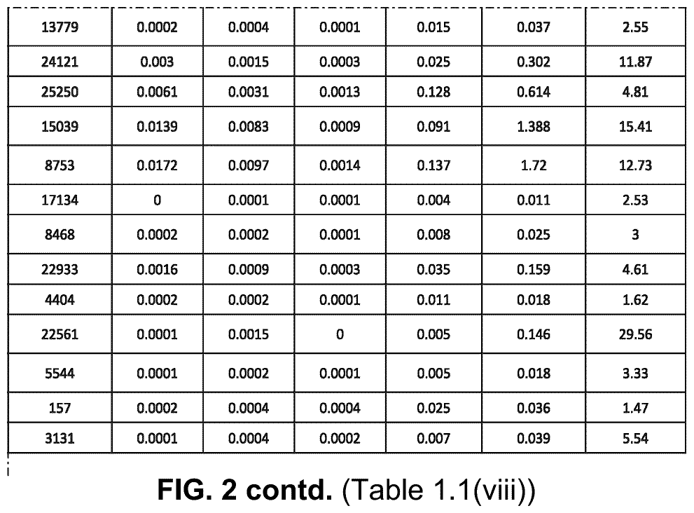

FIG. 2 (Table 1.1) provides bacterial taxa (OTUs) associated with health in mammals and with utility for the detection of health in cats.

TABLE 1.2
DNA sequences for bacterial taxa with utility for the assessment of health
in the gastrointestinal microbiome in cats.

| Cat | | |
|---|---|---|
| Denovo | SEQ | |
| OTU_ID | ID NO | 16SrDNA partial DNA sequence |
| 17970 | 3 | CCTGTTCGCTCCCCACGCTTTCGCTCCTCAGCGTCAGTAA |
| | | CGGCCCAGAGACCTGCCTTCGCCATTGGTGTTCTTCCCGA |
| | | TATCTACACATTCCACCGTTACACCGGGAATTCCAGTCTC |
| | | CCCTACCGCACTCAAGCCCGCCCGTACCCGGCGCGGATCC |
| | | ACCGTTAAGCGATGGACTTTCACACCGGACGCGACGAACC |
| | | GCCTACGAGCCCTTTACGCCCAATAATTCCGGATAACGCT |
| | | TGCACCCTACGTATTACCGCGGCTGCTGGCACGTAGTTAG |
| | | CCGGTGCTTATTCAACGGGTAAACTCACTCTCGCTTGCTC |
| | | CCCGATAAAAGAGGTTTACAACCCGAAGGCCTCCATCCCT |
| | | CACGCGGCGTCGCTGCATCAGGCTTGCGCCCATTGTGCAA |
| | | TATTCCCCA |
| 15408 | 4 | CCTGTTCGCTCCCCACGCTTTCGCTCCTCAGCGTCAGTGA |
| | | CGGCCCAGAGACCTGCCTTCGCCATTGGTGTTCTTCCCGA |
| | | TATCTACACATTCCACCGTTACACCGGGAATTCCAGTCTC |
| | | CCCTACCGCACTCAAGCCCGCCCGTACCCGGCGCGGATCC |
| | | ACCGTTAAGCGATGGACTTTCACACCGGACGCGACGAACC |
| | | GCCTACGAGCCCTTTACGCCCAATAATTCCGGATAACGCT |
| | | TGCACCCTACGTATTACCGCGGCTGCTGGCACGTAGTTAG |
| | | CCGGTGCTTATTCGAAAGGTACACTCACCCCGAAGGGCTT |
| | | GCTCCCAGTCAAAAGCGGTTTACAACCCGAAGGCCGTCAT |
| | | CCCGCACGCGGCGTCGCTGCATCAGGCTTCCGCCCATTGT |
| | | GCAAGATTCCCCA |
| 25425 | 5 | CCTGTTCGCTCCCCACGCTTTCGCTCCTCAGCGTCAGTAA |
| | | CGGCCCAGAGACCTGCCTTCGCCATTGGTGTTCTTCCCGA |
| | | TATCTACACATTCCACCGTTACACCGGGAATTCCAGTCTC |
| | | CCCTACCGCACTCCAGCCCGCCCGTACCCGGCGCAGATCC |
| | | ACCGTTAAGCGATGGACTTTCACACCGGACGCGACGAACC |
| | | GCCTACGAGCCCTTTACGCCCAATAATTCCGGATAACGCT |
| | | TGCACCCTACGTATTACCGCGGCTGCTGGCACGTAGTTAG |
| | | CCGGTGCTTATTCGAAAGGTACACTCACCCGAAGGCTTGC |
| | | TCCCGATCAAAAGCGGTTTACAACCCGAAGGCCGTCATCC |
| | | CGCACGCGGCGTCGCTGCATCAGGCTTGCGCCCATTGTGC |
| | | AATATTCCCCA |
| 8197 | 6 | CCTGTTCGCTCCCCACGCTTTCGCTCCTCAGCGTCAGTAA |
| | | CGGCCCAGAGACCTGCCTTCGCCATTGGTGTTCTTCCCGA |
| | | TATCTACACATTCCACCGTTACACCGGGAATTCCAGTCTC |
| | | CCCTACCGCACTCCAGCCCGCCCGTACCCGGCGCAGATCC |
| | | ACCGTTAAGCGATGGACTTTCACACCGGACGCGACGAACC |
| | | GCCTACGAGCCCTTTACGCCCAATAATTCCGGATAACGCT |
| | | TGCACCCTACGTATTACCGCGGCTGCTGGCACGTAGTTAG |
| | | CCGGTGCTTATTCGAAAGGTACACTCACCCGAAGGCTTGC |
| | | TCCCGATCAAAAGCGGTTTACAACCCGAAGGCCTCCGTCC |
| | | CGCACGCGGCGTCGCTGCGTCAGGGTTCCCCCCATTGCGC |
| | | AAGATTCCCCA |
| 24278 | 7 | CCTGTTCGCTCCCCACGCTTTCGCTCCTCAGCGTCAGTAA |
| | | CGGCCCAGAGACCTGCCTTCGCCATTGGTGTTCTTCCCGA |
| | | TATCTACACATTCCACCGTTACACCGGGAATTCCAGTCTC |
| | | CCCTGCCGCACTCCAGCCCGCCCGTACCCGGCGCAGATCC |
| | | ACCGTTAAGCGATGGACTTTCACACCAGACGCGACGAACC |
| | | GCCTACGAGCCCTTTACGCCCAATAAATCCGGATAACGCT |
| | | TGCACCCTACGTATTACCGCGGCTGCTGGCACGTAGTTAG |
| | | CCGGTGCTTATTCAACGGGTACACTCACTCTCGCTTGCTC |
| | | CCCGATAAAAGCGGTTTACAACCCGAAGGCCTCCATCCCG |
| | | CACGCGGCGTCGCTGCGTCAGGCTTTCGCCCATTGCGCAA |
| | | TATTCCCCA |
| 4717 | 8 | CCTGTTCGCTCCCCCAGCTTTCGCGCCTCAGCGTCGGTCA |
| | | CGGCCCAGAGGGCCGCCTTCGCCACCGGTGTTCCACCCGA |
| | | TATCTGCGCATTCCACCGCTACACCGGGTGTTCCACCCTC |
| | | CCCTACCGGACCCAAGCCCGGAGGTTCCGGGGGCGGGCGG |
| | | GGGTTGAGCCCCCGCATTTGACCCCCGGCCTGCCAGGCCG |
| | | CCTACGCGCGCTTTACGCCCAATGAATCCGGATAACGCTC |
| | | GCCCCCTACGTATTACCGCGGCTGCTGGCACGTAGTTAGC |
| | | CGGGGCTTCTTCTGCAGGTACAGTCTTGTCTCTTCCCTGC |
| | | TGAAAGCGGTTTACGACCCGAAGGCCTCCGTCCCGCACGC |
| | | GGCGTCGCTGCGTCAGGGTTGCCCCCATTGCGCAAGATTC |
| | | CCCA |
| 21150 | 9 | CCTGTTCGCTCCCCCAGCTTTCGCGCCTCAGCGTCGGTCT |
| | | CGGCCCAGAGGGCCGCCTTCGCCACCGGTGTTCCTCCCGA |
| | | TATCTGCGCATTCCACCGCTACACCGGGAATTCCACCCTC |
| | | CCCTACCGGACCCGAGCCGCGGGGTTCGGGGGGCGGACCG |
| | | GGGTTGAGCCCCGGGATTTGACCCCCCGCCTACGCGGCCG |
| | | CCTACGCGCGCTTTACGCCCAATGAATCCGGATAACGCTC |
| | | GCCCCCTACGTATTACCGCGGCTGCTGGCACGTAGTTAGC |
| | | CGGGGCTTCTTCTGCAGGTACAGTCTTGTCTCTTCCCTGC |
| | | TGAAAGCGGTTTACGACCCGAAGGCCTCCATCCCGCACGC |
| | | GGCGTCGCTGCGTCAGGGTTGCCCCCATTGCGCAAGATTC |
| | | CCCA |
| 4018 | 10 | CCTGTTCGCTCCCCCAGCTTTCGCGCCTCAGCGTCGGTCT |
| | | CGGCCCAGAGGGCCGCCTTCGCCACCGGTGTTCCTCCCGA |
| | | TATCTGCGCATTCCACCGCTACACCGGGAATTCCACCCTC |
| | | CCCTACCGGACCCGAGCCGCGGGGTTCGGGGGGCGGACCG |
| | | GGGTTGAGCCCCGGGATTTGACCCCCCGCCTACGCGGCCG |
| | | CCTACGCGCGCTTTACGCCCAATGAATCCGGATAACGCTC |
| | | GCCCCCTACGTATTACCGCGGCTGCTGGCACGTAGTTAGC |
| | | CGGGGCTTCTTCTGCGGGTACCGTCTTGACTCGTCCCCGC |
| | | TGAAAGCGGTTTACGACCCGAAGGCCTTCGTCCCGCACGC |
| | | GGCGTCGCTGCGTCAGGGTTTCCCCCATTGCGCAATATTC |
| | | CCCA |
| 5780 | 11 | CCTGTTCGCTCCCCCAGCTTTCGCGCCTCAGCGTCGGTCT |
| | | CGGCCCAGAGGGCCGCCTTCGCCACCGGTGTTCCACCCGA |
| | | TATCTGCGCATTCCACCGCTACACCGGGTGTTCCACCCTC |
| | | CCCTACCGGACCCAAGCCGCGGAGGTTCCGGGGGCTTCGG |
| | | GGGGTTGAGCCCCCCGCTTCGACCCCCGGCCTGCCGGGCC |
| | | GCCTACGCGCGCTTTACGCCCAATGAATCCGGATAACGCT |
| | | CGCCCCCTACGTATTACCGCGGCTGCTGGCACGTAGTTAG |
| | | CCGGGGCTTCTTCTGCAGGTACAGTCTTGACTCTTCCCTG |
| | | CTGAAAGCGGTTTACGACCCGAAGGCCTCCGTCCCGCACG |
| | | CGGCGTCGCTGCGTCAGGGTTCCCCCCATTGCGCAAGATT |
| | | CCCCA |
| 5891 | 12 | CCTGTTCGCTCCCCCAGCTTTCGCGCCTCAGCGTCGGTCT |
| | | CGGCCCAGAGGGCCGCCTTCGCCACCGGTGTTCCACCCGA |
| | | TATCTGCGCATTCCACCGCTACACCGGGTGTTCCACCCTC |
| | | CCCTACCGGACCCGAGCCGCGGGGTTCGGGGGGCGGACCG |
| | | GGGTTGAGCCCCGGGATTTGACCCCCCGCCTACGCGGCCG |
| | | CCTACGCGCGCTTTACGCCCAATGAATCCGGATAACGCTC |
| | | GCCCCCTACGTATTACCGCGGCTGCTGGCACGTAGTTAGC |
| | | CGGGGCTTCTTCTGCAGGTACCGTCTTGCCTCATCCCTGC |
| | | TGAAAGCGGTTTACGACCCGAGGGCCTTCGTCCCGCACGC |
| | | GGCGTCGCTGCGTCAGGGTTCCCCCCATTGCGCAAGATTC |
| | | CCCA |
| 23268 | 13 | CCTGTTCGCTCCCCCAGCTTTCGCTCCTCAGCGTCAGTGA |
| | | TGGCCCAGAAAGCCGCCTTCGCCACCGGTGTTCTTCCCGA |
| | | TATCTGCGCATTCCACCGCTACACCGGGAATTCCGCTTTC |
| | | CCCTACCACACTCAAGTCCACCGGTATCGGGGGCGGACCG |
| | | GGGTTGAGCCCCGGGATTTGACCTCCGACCTAATGGACCG |
| | | CCTACGAGCGCTTTACGCCCAATGAATCCGGATAACGCTC |
| | | GCCCCCTACGTATTACCGCGGCTGCTGGCACGTAGTTAGC |
| | | CGGGGCTTCTTCTGCAGGTACCGTCACTTTCGCCTCGTCC |
| | | CTGCTGAAAGCGGTTTACGACCCGAAGGCCTTCATCCCGC |
| | | ACGCGGCGTCGCTGCGTCAGGCTTTCGCCCATTGCGCAAG |
| | | ATTCCCCA |
| 26033 | 14 | CCTGTTTGCTCCCCACGCTTTCGTGCCTCAGCGTCAGTTA |
| | | CAGTCCAGAAAGCCGCCTTCGCTACTGGTGTTCCTCCCAA |
| | | TATCTACGCATTTCACCGCTACACTGGGAATTCCGCTTTC |
| | | CTCTCCTGCACTCAAGTCAGACAGTATCAGGAGCTTACTA |
| | | CGGTTGAGCCGTAGCCTTTAACTCCTGACTTGAAAGACCG |
| | | CCTACGCACCCTTTACGCCCAGTAAATCCGGATAACGCTA |
| | | GCCCCCTACGTATTACCGCGGCTGCTGGCACGTAGTTAGC |
| | | CGGGGCTTCCTCCTCAAGTACCGTCATTATCTTCCTTGAG |
| | | GACAGAGCTTTACGACCCGAAGGCCTTCATCGCTCACGCG |
| | | GCGTTGCTGCATCAGGCTTGCGCCCATTGTGCAATATTCC |
| | | CCA |
| 6859 | 15 | CCTGTTTGCTCCCCACGCTTTCGTGCCTCAGCGTCAGTTC |
| | | CAGTCCAGCAGGCCGCCTTCGCCACTGGTGTTCCTCCTAA |
| | | TATCTACGCATTTCACCGCTACACTAGGAATTCCGCCTGC |
| | | CTCTCCTGGACTCCAGCAAAACAGTTTCAAATGCAGCCCC |
| | | GGAGTTGAGCCCCGGTCTTTCACATCTGACTTGCCTTGCC |
| | | GCCTACGCACCCTTTACACCCAGTAAATCCGGATAACGCT |
| | | TGCTCCATACGTATTACCGCGGCTGCTGGCACGTATTTAG |
| | | CCGGAGCTTCTTAGTCAGGTACCGTCATTATCTTCCCTGC |
| | | TGATAGAGCTTTACATACCGAAATACTTCTTCACTCACGC |
| | | GGCGTTGCTGCATCAGGGTTTCCCCCATTGTGCAATATTC |
| | | CCCA |
| 28662 | 16 | CCTGTTTGCTCCCCACGCTTTCGTACCTCAGTGTCAGTTA |
| | | CAGTCCAGAAAGCCGCCTTCGCCACTGGTGTTCCTCCTAA |
| | | TATCTACGCATTTCACCGCTACACTAGGAATTCCGCTTTC |
| | | CTCTCCTGCACTCAAGTTTGCCAGTTCGCAGGGCGAACAA |
| | | TGGTTGAGCCATTGCCTTAAACCCTGCGCTTGGTAAACCA |
| | | CCTACGTACCCTTTACGCCCAATAATTCCGGATAACGCTT |
| | | GCCCCCTCCGTATTACCGCGGCTGCTGGCACGGAGTTAGC |
| | | CGGGGCTTCCTCCAAGGGTACCGTCATTTGTTTCTTCCCC |
| | | AAGGACAGAGCTTTACGACCCAAAGGCCTTCATCGCTCAC |
| | | GCGGCGTTGCTGCATCAGGCTTGCGCCCATTGTGCAATAT |
| | | TCCCCA |
| 22810 | 17 | CCTGTTTGCTCCCCACGCTTTCGTGCCTCAGTGTCAGTTT |
| | | CAGTCCAGTAAGCCGCCTTCGCCACTGATGTTCCTCCTAA |
| | | TATCTACGCATTTCACCGCTACACTAGGAATTCCGCTTAC |
| | | CTCTCCTGCACTCCAGTTCGACAGTTTCAAAAGCAGTCCC |
| | | GCGGTTAAGCCCCGGGCTTTCACTTCTGACTTGCCGTACC |
| | | ACCTACGCACCCTTTACACCCAGTAATTCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAGCTTTACATACCGAAATACTTCTTCACTCACGC |
| | | GGCGTCGCTGCATCAGGGTTCCCCCCATTGTGCAATATTC |
| | | CCCA |
| 6362 | 18 | CCTGTTTGCTACCCACGCTTTCGAGCCTCAGCGTCAGTTA |
| | | CAGCCCAGTAGGCCGCCTTCGCCACTGGTGTTCCTCCTAA |
| | | TATCTACGCATTTCACCGCTACACTAGGAATTCCGCCTAC |
| | | CTCTACTGCACTCAAGAGTGGCAGTTTTGAACGCGACTAT |
| | | CAGTTGAGCCGATAGTTTAAACATTCAACTTGCCTCCCCG |
| | | CCTACGCTCCCTTTACACCCAGTAATTCCGGACAACGCTT |
| | | GCCACCTACGTATTACCGCGGCTGCTGGCACGTAGTTAGC |
| | | CGTGGCTTCCTCTTTAGGTACCGTCATTTTTTTCGTCCCT |
| | | AAAGACAGAGGTTTACAATCCGAAAACCGTCTTCCCTCAC |
| | | GCGGCGTCGCTGCATCAGGGTTCCCCCCATTGTGCAATAT |
| | | CCCCCA |
| 6728 | 19 | CCTGTTTGCTCCCCACGCTTTCGAGCCTCAACGTCAGTCA |
| | | TCGTCCAGAAAGCCGCCTTCGCCACTGGTGTTCCTCCTAA |
| | | TATCTACGCATTTCACCGCTACACTAGGAATTCCGCTTTC |
| | | CTCTCCGACACTCTAGCCTGACAGTTCCAAATGCAGTCCC |
| | | GGGGTTGAGCCCCGGGCTTTCACATCTGGCTTGCCATGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAGCTTTACATACCGAAATACTTCATCGCTCACGC |
| | | GGCGTCGCTGCATCAGGGTTTCCCCCATTGTGCAATATTC |
| | | CCCA |
| 11072 | 20 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CCCTCCGGCACTCAAGTATGACAGTTTCCAATGCAGTCCA |
| | | CAGGTTGAGCCCATGCCTTTCACATCAGACTTGCCACACC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCACTATCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCCA |
| 25796 | 21 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCGAGTATCACAGTTTCCAATGCAGTCCA |
| | | GGGGTTGAGCCCCCGCCTTTCACATCAGACTTGCAACACC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCCA |
| 16545 | 22 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGTACTCAAGATCAACAGTTTCCAATGCAGTCCG |
| | | GGGGTTGAGCCCCCGCCTTTCACATCAGACTTGCTGCTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCCA |
| 23005 | 23 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCTAGAAAAACAGTTTCCAATGCAGTCCT |
| | | GGGGTTAAGCCCCAGCCTTTCACATCAGACTTGCTCTTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCCA |
| 18810 | 24 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCCAGATGGACAGTTTCCAAAGCAGTCCA |
| | | GGGGTTGAGCCCCTGCCTTTCACTTCAGACTTGCCCGTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTGTCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCTA |
| 16620 | 25 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CCCTCCGGTACTCAAGACTGACAGTTTCCAATGCAGTCCA |
| | | GGGGTTGAGCCCCTGCCTTTCACATCAGACTTGCCATTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAGATACTTCTTCCTTCACGC |
| | | GGCGTCGCTGCATCAGGGTTTCCCCCATTGTGCAATATTC |
| | | CCCA |
| 3067 | 26 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCAAGATGGACAGTTTCCAATGCAGTCCC |
| | | GGGGTTAAGCCCCGGGCTTTCACATCAGACTTGCCCGTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAGATACTTCTTCCTTCACGC |
| | | GGCGTCGCTGCATCAGAGTTTCCTCCATTGTGCAATATTC |
| | | CCCA |
| 22167 | 27 | CCTGTTTGCTCCCCACGCTTTCGCGCCTCAGCGTCAGTTA |
| | | ATGTCCAGCAGGCCGCCTTCGCCACTGGTGTTCCTCCGTA |
| | | TATCTACGCATTTCACCGCTACACACGGAATTCCGCCTGC |
| | | CTCTCCATCACTCAAGACCAGCAGTTTTGAAAGCAGTTCG |
| | | GGGGTTAAGCCCCCGGATTTCACTCCCAACTTGCCGGCCC |
| | | GCCTGCGCGCCCTTTACACCCAGTAAATCCGGATAACGCT |
| | | TGCTCCCTACGTATTACCGCGGCTGCTGGCACGTAGTTAG |
| | | CCGGAGCTTATTCTTCAGGTACCGTCATTCTTCGTCCCTG |
| | | ATTAAAGATCTTTACAACCCGAAGGCCTTCTTCAATCACG |
| | | CGGCGTTGCTGCGTCAGGGTTTCCCCCATTGCGCAATATT |
| | | CCCCA |
| 15942 | 28 | CCTATTTGCTCCCCACGCTTTCGGGAATGAGCGTCAGTTG |
| | | CGGCCCAGACCGTCGCCTTCGCCACTGGTGTTCCTCCATA |
| | | TATCTACGCATTTCACCGCTACACATGGAATTCCACGATC |
| | | CTCTTCCGCACTCTAGCCGCCCGGTTTCCATGGCTTACCG |
| | | AAGTTTAGCTTCGGTCTTTCACCACAGACCCTTGCGGCCG |
| | | CCTGCTCCCTCTTTACGCCCAATGATTCCGGATAACGCTC |
| | | GCCACCTACGTATTACCGCGGCTGCTGGCACGTAGTTAGC |
| | | CGTGGCTTTCTGGTAAAGTACCGTCACTCGCATACCATTT |
| | | CCTGTATGCGCCGTTCTTCCTTTACAACAGAGCTTTACGA |
| | | CCCGAAGGCCTTCTTCGCTCACGCGGCGTTGCTCGGTCAG |
| | | GGTTTCCCCCATTGCCGAAAATTCCCTA |
| 18957 | 29 | CCTGTTTGCTCCCCACGCTTTCGAGCCTCAGCGTCAGTTA |
| | | CAGTCCAGAGAGTCGCCTTCGCCACTGGTGTTCTTCCTAA |
| | | TCTCTACGCATTTCACCGCTACACTAGGAATTCCACTCTC |
| | | CTCTCCTGCACTCTAGATAATCAGTTTGGAATGCAGCCCC |
| | | CAGGTTGAGCCTGAGTATTTCACATCCCACTTAATTATCC |
| | | GCCTACGCTCCCTTTACGCCCAATAATTCCGGATAACGCT |
| | | CGCCACCTACGTATTACCGCGGCTGCTGGCACGTAGTTAG |
| | | CCGTGGCTTCCTCCTTGGGTACCGTCATTATCGTCCCCAA |
| | | AGACAGAGCTTTACAATCCGAAGACCGTCATCACTCACGC |
| | | GGCGTTGCTGCATCAGGGTTTCCCCCATTGTGCAATATTC |
| | | CCCA |
| 27916 | 30 | CCTGTTTGCTACCCACACTTTCGAGCCTCAGCGTCAGTTG |
| | | GTGCCCAGTAGGCCGCCTTCGCCACTGGTGTTCCTCCCGA |
| | | TATCTACGCATTCCACCGCTACACCGGGAATTCCGCCTAC |
| | | CTCTGCACTACTCAAGAAAAACAGTTTTGAAAGCAGTTTA |
| | | TGGGTTGAGCCCATAGATTTCACTTCCAACTTGTCTTCCC |
| | | GCCTGCGCTCCCTTTACACCCAGTAATTCCGGACAACGCT |
| | | TGTGACCTACGTTTTACCGCGGCTGCTGGCACGTAGTTAG |
| | | CCGTCACTTCCTTGTTGGGTACCGTCATTATCTTCCCCAA |
| | | CAACAGGAGTTTACAATCCGAAGACCTTCTTCCTCCACGC |
| | | GGCGTCGCTGCATCAGGGTTTCCCCCATTGTGCAATATTC |
| | | CCCA |
| 6662 | 31 | CCTATTTGCTCCCCACGCTTTCGTGCTTCAGTGTCAGAAT |
| | | CCAGACCAGACGGCCGCCTTCGCCACCGGTGTTCTTCCAT |
| | | ATATCTACGCATTTTACCGCTACACATGGAGTTCCGCCGT |
| | | CCTCTTCTGTTCTCTAGCTGATCAGTTTCCAGAGCAAGTA |
| | | CGGGTTGAGCCCATACCTTTTACTCCAGACTTGATCTGCC |
| | | ACCTACGCACCCTTTACGCCCAATCATTCCGGATAACGCT |
| | | CGCCACCTACGTATTACCGCGGCTGCTGGCACGTAGTTAG |
| | | CCGTGACTTTCTGGTAAGATACCATCACTCACTCATCATT |
| | | CCCTATGAGTGCCGTTTTTCTCTTACAACAGAGCTTTACG |
| | | ATCCGAAGACCTTCCTCACTCACGCGGCATTGCTCGTTCA |
| | | GGGTTCCCCCCATTGACGAAAATTCCCTA |
| 17107 | 32 | CCTGTTTGCTCCCCACGCTTTCGAGCCTCAACGTCAGTTA |
| | | CTGTCCAGTAAGCCGCCTTCGCCACTGGTGTTCCTCCTAA |
| | | TATCTACGCATTTCACCGCTACACTAGGAATTCCACTTAC |
| | | CTCTCCAGCACTCTAGCAACACAGTTTCCAAAGCAGTCCC |
| | | AGGGTTGAGCCCTGGGCTTTCACTTCAGACTTGCATCGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCTTTCTTCTTCCCTG |
| | | CTGATAGAGCTTTACATACCGAAATACTTCTTCACTCACG |
| | | CGGCGTCGCTGCATCAGGGTTTCCCCCATTGTGCAATATT |
| | | CCCCA |
| 20134 | 33 | CCTGTTTGCTCCCCACGCTTTCGAGCCTCAACGTCAGTTA |
| | | CTGTCCAGTAAGCCGCCTTCGCCACTGGTGTTCCTCCTAA |
| | | TATCTACGCATTTCACCGCTACACTAGGAATTCCACTTAC |
| | | CTCTCCAGCACTCAAGCTACACAGTTTCCAAAGCAGTCCC |
| | | GGGGTTGAGCCCCGGGCTTTCACTTCAGACTTGCACAGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCATCCTTCACGC |
| | | GGCGTCGCTGCATCAGGGTTTCCCCCATTGTGCAATATTC |
| | | CCCA |
| 17276 | 34 | CCTGTTTGCTCCCCACGCTTTCGAGCCTCAACGTCAGTTA |
| | | TCGTCCAGTAAGCCGCCTTCGCCACTGATGTTCCTCCTAA |
| | | TATCTACGCATTTCACCGCTACACTAGGAATTCCGCTTAC |
| | | CTCTCCGACACTCTAGAAGCACAGTTTCCAAAGCAGTCAC |
| | | GGGGTTGAGCCCCGGGCTTTCACTTCAGACTTGCACTTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCTTCCTTCACGC |
| | | GGCGTCGCTGCATCAGGGTTTCCCCCATTGTGCAATATTC |
| | | CCCA |
| 4544 | 35 | CCTGTTTGCTCCCCACGCTTTCGAGCCTCAACGTCAGTTA |
| | | CTGTCCAGTAAGCCGCCTTCGCCACTGGTGTTCCTCCTAA |
| | | TATCTACGCATTTCACCGCTACACTAGGAATTCCGCTTAC |
| | | CTCTCCAGCACTCCAGCTTAACAGTTTCCAAAGCAGTCCC |
| | | GGGGTTGAGCCCCGGGCTTTCACTTCAGACTTGCTAAGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTCTCTTCCCTGC |
| | | TGATAGAGCTTTACATACCGAAATACTTCTTCACTCACGC |
| | | GGCGTCGCTGCATCAGGGTTTCCCCCATTGTGCAATATTC |
| | | CCCA |
| 15603 | 36 | CCTGTTTGCTCCCCACGCTTTCGAGCCTCAACGTCAGTCA |
| | | TCGTCCAGTAAGCCGCCTTCGCCACTGGTGTTCCTCCTAA |
| | | TATCTACGCATTTCACCGCTACACTAGGAATTCCACTTAC |
| | | CTCTCCGACACTCTAGAAAAACAGTTTCCAATGCAGTCCC |
| | | GGGGTTGAGCCCCGGGTTTTCACATCAGACTTGCCTCTCC |
| | | GTCTACGCTCCCTTTACACCCAGTAAATCCGGATAACGCT |
| | | TGCACCATACGTATTACCGCGGCTGCTGGCACGTATTTAG |
| | | CCGGTGCTTCTTAGTCAGGTACCGTCATTTTCTTCCCTGC |
| | | TGATAGAGCTTTACATACCGAAATACTTCATCGCTCACGC |
| | | GGCGTCGCTGCATCAGGGTTTCCCCCATTGTGCAATATTC |
| | | CCCA |
| 24567 | 37 | CCTGTTTGCTCCCCACGCTTTCGAGCCTCAACGTCAGTTA |
| | | TCGTCCAGCAAGCCGCCTTCGCCACTGGTGTTCCTCCTAA |
| | | TATCTACGCATTTCACCGCTACACTAGGAATTCCGCTTGC |
| | | CTCTCCGACACTCCAGCTGCACAGTTTCCAAAGCAGTCCC |
| | | GGGGTTGAGCCCCGGGCTTTCACTTCAGACTTGCACTGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCATCCTTCACGC |
| | | GGCGTCGCTGCATCAGGGTTTCCCCCATTGTGCAATATTC |
| | | CCCA |
| 13207 | 38 | CCTGTTTGCTCCCCACGCTTTCGTGCCTCAGTGTCAGTAA |
| | | CAGTCCAGCAGGCCGCCTTCGCCACTGGTGTTCCTCCTAA |
| | | TATCTACGCATTTCACCGCTACACTAGGAATTCCGCCTGC |
| | | CTCTCCTGTACTCTAGCCGAGCAGTTTCAAATGCAGCTCC |
| | | GGGGTTGAGCCCCGGCCTTTCACATCTGACTTGCACTGCC |
| | | ACCTACGCACCCTTTACACCCAGTAAATCCGGATAACGCT |
| | | TGCTCCATACGTATTACCGCGGCTGCTGGCACGTATTTAG |
| | | CCGGAGCTTCTTAGTCAGGTACCGTCATTATCTTCCCTGC |
| | | TGATAGAGCTTTACATACCGAAATACTTCTTCACTCACGC |
| | | GGCGTTGCTGCATCAGGGTTTCCCCCATTGTGCAATATTC |
| | | CCCA |
| 17701 | 39 | CCTGTTTGCTCCCCACGCTTTCGAGCCTCAACGTCAGTTA |
| | | TCGTCCAGTGAGCCGCCTTCGCCACTGGTGTTCCTCCTAA |
| | | TATCTACGCATTTCACCGCTACACTAGGAATTCCACTCAC |
| | | CTCTCCGACACTCTAGTTATACAGTTTCCAATGCAGGTCC |
| | | AGGGTTAAGCCCTGGAATTTCACATCAGACTTGCACAACC |
| | | GTCTACGCTCCCTTTACACCCAGTAAATCCGGATAACGCT |
| | | TGCTCCATACGTATTACCGCGGCTGCTGGCACGTATTTAG |
| | | CCGGAGCTTCTTAGTCAGGTACCGTCATTATCTTCCCTGC |
| | | TGATAGAGCTTTACATACCGAAATACTTCTTCGCTCACGC |
| | | GGCGTCGCTGCATCAGGGTTTCCCCCATTGTGCAATATTC |
| | | CCCA |
| 24239 | 40 | CCTGTTTGCTCCCCACGCTTTCGAGCCTCAACGTCAGTTA |
| | | TCGTCCAGTAAGCCGCCTTCGCCACTGGTGTTCCTCCTAA |
| | | TATCTACGCATTTCACCGCTACACTAGGAATTCCACTTAC |
| | | CCCTCCGACACTCTAGACTGACAGTTTCCAATGCAGTCCC |
| | | GGGGTTGAGCCCCGGGTTTTCACATCAGACTTGCCAGTCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAGCTTTACATACCGAAATACTTCTTCGCTCACGC |
| | | GGCGTCGCTGCATCAGGGTTTCCCCCATTGTGCAATATTC |
| | | CCCA |
| 2154 | 41 | CCTGTTTGCTCCCCACGCTTTCGTGCCTCAGCGTCAGTTA |
| | | CAGTCCAGAAAGCCGCCTTCGCTACTGGTGTTCCTCCCAA |
| | | TATCTACGCATTTCACCGCTACACTGGGAATTCCGCTTTC |
| | | CTCTCCTGCACTCAAGTCAGACAGTATCAGGAGCTTACTA |
| | | CGGTTGAGCCGTAGCCTTTAACTCCTGACTTGAAAGACCG |
| | | CCTACGCACCCTTTACGCCCAGTAAATCCGGATAACGCTA |
| | | GCCCCCTACGTATTACCGCGGCTGCTGGCACGTAGTTAGC |
| | | CGGGGCTTCTTAGTCAGGTACCGTCATTTTCTTCCCTGCT |
| | | GATAGAAGTTTACATACCGAGATACTTCTTCCTTCACGCG |
| | | GCGTCGCTGCATCAGGGTTTCCCCCATTGTGCAATATTCC |
| | | CCA |
| 1809 | 42 | CCTGTTTGCTCCCCACGCTTTCGAGCCTCAACGTCAGTTA |
| | | CTGTCCAGTAAGCCGCCTTCGCCACTGGTGTTCCTCCTAA |
| | | TATCTACGCATTTCACCGCTACACTAGGAATTCCGCTTAC |
| | | CTCTCCAGTACTCTAGCCGCACAGTTTCCAATGCAGTCCC |
| | | GGGGTTGAGCCCCGGGCTTTCACATCAGACTTGCACCGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCTTCCTTCACGC |
| | | GGCGTCGCTGCATCAGGGTTTCCCCCATTGTGCAATATTC |
| | | CCCA |
| 5249 | 43 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCCAGCAGAACAGTTTCCAATGCAGGTCC |
| | | GGGGTTGAGCCCCGGAATTTCACATCAGACTTGCTCCGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTATCTTCCCTGC |
| | | TGATAGAGCTTTACATACCGAAATACTTCTTCGCTCACGC |
| | | GGCGTCGCTGCATCAGGGTTTCCCCCATTGTGCAATATTC |
| | | CCCA |
| 3133 | 44 | CCTGTTTGCTCCCCACGCTTTCGAGCCTCAACGTCAGTTA |
| | | CCGTCCAGTAAGCCGCCTTCGCCACTGGTGTTCTTCCTAA |
| | | TATCTACGCATTTCACCGCTACACTAGGAATTCCGCTTAC |
| | | CTCTCCGGCACTCCAGCTAAACAGTTTCCAAAGCAGTCCC |
| | | GGCGTTAAGCACCGGGCTTTCACTTCAGACTTGCCTTGCC |
| | | GTCTACACTCCCTTTACACCCAGTAAATCCGGATAACGCT |
| | | TGCCCCCTACGTATTACCGCGGCTGCTGGCACGTAGTTAG |
| | | CCGGGGCTTCTTATTCAGGTACCGTCACTTTCTTCCCTGC |
| | | TGATAGAGCTTTACATACCGAAATACTTCTTCACTCACGC |
| | | GGCGTCGCTGCATCAGGGTTTCCCCCATTGTGCAATATTC |
| | | CCCA |
| 3856 | 45 | CCTGTTTGCTCCCCACGCTTTCGAGCCTCAACGTCAGTTA |
| | | CTGTCCAGTAAGCCGCCTTCGCCACTGGTGTTCCTCCTAA |
| | | TATCTACGCATTTCACCGCTACACTAGGAATTCCACTTAC |
| | | CTCTCCAGCACTCTAGCAGAACAGTTTCCAAAGCAGTCCC |
| | | GGGGTTGAGCCCCGGGCTTTCACTTCAGACTTGCTCCGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAGCTTTACATACCGAGATACTTCTTCACTCACGC |
| | | GGCGTCGCTGCATCAGGGTTTCCCCCATTGTGCAATATCC |
| | | CCCA |
| 21025 | 46 | CCTGTTTGCTCCCCACGCTTTCGAGCCTCAACGTCAGTTA |
| | | CTGTCCAGCAAGCCGCCTTCGCCACTGGTGTTCCTCCTAA |
| | | TATCTACGCATTTCACCGCTACACTAGGAATTCCGCTTGC |
| | | CTCTCCAGCACTCCAGCCCAACAGTTTCCAAAGCAGTTCC |
| | | CGGGTTGAGCCCGGGGATTTCACTTCAGACTTGCTGTGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCACTTTCTTCCCTGC |
| | | TGATAGAGCTTTACATACCGAAATACTTCTTCACTCACGC |
| | | GGCGTTGCTGCATCAGGGTTTCCCCCATTGTGCAATATTC |
| | | CCCA |
| 17911 | 47 | CCTGTTTGCTACCCACGCTTTCGAACCTCAGCGTCAGTTA |
| | | CAGACCAGAGAGCCGCTTTCGCCACTGGTGTTCTTCCATA |
| | | TATCTACGCATTTCACCGCTACACATGGAGTTCCACTCTC |
| | | CTCTTCTGCACTCAAGTCTCCCAGTTTCCAATGCACTACT |
| | | CCGGTTAAGCCGAAGGCTTTCACATCAGACTTAAAAGACC |
| | | GCCTGCGTTCCCTTTACGCCCAATAAATCCGGATAACGCT |
| | | TGCCACCTACGTATTACCGCGGCTGCTGGCACGTAGTTAG |
| | | CCGTGGCTTTCTGGTTAGATACCGTCGAAACGTGAACAGT |
| | | TACTCTCACGCACTTTCTTCTCTAACAACAGGGTTTTACG |
| | | ATCCGAAGACCTTCTTCACCCACGCGGCGTTGCTCCATCA |
| | | GGCTTTCGCCCATTGTGGAAGATTCCCTA |
| 21633 | 48 | CCTGTTCGCTACCCATGCTTTCGAGCCTCAGCGTCAGTTG |
| | | CAGACCAGACAGCCGCCTTCGCCACTGGTGTTCTTCCATA |
| | | TATCTACGCATTCCACCGCTACACATGGAGTTCCACTGTC |
| | | CTCTTCTGCACTCAAGTCGCCCGGTTTCCGATGCACTTCT |
| | | TCGGTTAAGCCGAAGGCTTTCACATCAGACCTAAGCAACC |
| | | GCCTGCGCTCGCTTTACGCCCAATAAATCCGGATAACGCT |
| | | TGCCACCTACGTATTACCGCGGCTGCTGGCACGTAGTTAG |
| | | CCGTGACTTTCTGGTTGGATACCGTCACTGCGTGAACAGT |
| | | TACTCTCACGCACGTTCTTCTCCAACAACAGAGCTTTACG |
| | | AGCCGAAACCCTTCTTCACTCACGCGGTGTTGCTCCATCA |
| | | GGCTTGCGCCCATTGTGGAAGATTCCCTA |
| 21790 | 49 | CCTGTTTGCTACCCACGCTTTCGAACCTCAGCGTCAGTTA |
| | | CAGACCAGAGAGCCGCTTTCGCCACTGGTGTTCTTCCATA |
| | | TATCTACGCATTTCACCGCTACACATGGAGTTCCACTCTC |
| | | CTCTTCTGCACTCAAGTCTTCCAGTTTCCAATGCACTACT |
| | | TCGGTTAAGCCGAAGGCTTTCACATCAGACTTAAAAGACC |
| | | GCCTGCGTTCCCTTTACGCCCAATAAATCCGGACAACGCT |
| | | CGCCACCTACGTATTACCGCGGCTGCTGGCACGTAGTTAG |
| | | CCGTGGCTTTCTGGTCAGATACCGTCAATACGTGAACAGT |
| | | TACTCTCACGCACGTTCTTCTCTGACAACAGAATTTTACG |
| | | ACCCGAAGGCCTTCTTCATTCACGCGGCGTTGCTCCATCA |
| | | GACTTTCGTCCATTGTGGAAGATTCCCTA |
| 28608 | 50 | CCCGTTCGCTACCCTAGCTTTCGAGCCTCAGCGTCAGTTA |
| | | CAGTCCAGAAAGCCGCCTTCGCCACTGGTGTTCTTCCCAA |
| | | TATCTACGCATTTCACCGCTACACTGGGAATTCCGCTTTC |
| | | CTCTCCTGCACTCAAGAAAACCAGTTCGGACCCCATCACG |
| | | GGGTTGAGCCCCGCACTTTTAAGATCCGCTTAGTTTCCCG |
| | | CCTGCGCTCCCTTTACGCCCAATAATTCCGGACAACGCTT |
| | | GCCGCCTACGTATTACCGCGGCTGCTGGCACGTAGTTAGC |
| | | CGTGGCTTCCTCGTTAGGTACCGTCAAATAAAAACCTTAT |
| | | TATGATTCTTACCCTTCGTCCCTAACAACAGAACTTTACG |
| | | ATCCTAAGACCTTCATCGTTCACGCGGCGTTGCTCCGTCA |
| | | GGCTTTCGCCCATTGCGGAAGATTCCCCA |
| 28233 | 51 | CCCGTTCGCTACCCTAGCTTTCGAGCCTCAGCGTCAGTTA |
| | | CAGTCCAGAAAGCCGCCTTCGCCACTGGTGTTCTTCCCAA |
| | | TATCTACGCATTTCACCGCTACACTGGGAATTCCGCTTTC |
| | | CTCTCCTGCACTCAAGAAAATCAGTTCGGACCCCATCACG |
| | | AGGTTGAGCCCCGCACTTTTAAGATCCGCTTAATTTCCCG |
| | | CCTGCGCTCCCTTTACGCCCAATAATTCCGGACAACGCTT |
| | | GCCACCTACGTATTACCGCGGCTGCTGGCACGTAGTTAGC |
| | | CGTGGCTTCCTCGTTAAGTACCGTCAAATACTTACTGTAT |
| | | TATAATAAGCATCCTTCGCCCTTAACAACAGAACTTTACG |
| | | ATCCGAAGACCTTCTTCGTTCACGCGGCGTTGCTCCGTCA |
| | | GGCTTTCGCCCATTGCGGAAGATTCCCCA |
| 13977 | 52 | CCCGTTTGCTCCCCTGGCTTTCGCGCCTCAGCGTCAGTTG |
| | | TCGTCCAGAAAGCCGCTTTCGCCACTGGTGTTCCTCCTAA |
| | | TATCTACGCATTTCACCGCTACACTAGGAATTCCGCTTTC |
| | | CTCTCCGATACTCTAGCATCGCAGTTTCGGTCCCCTCACG |
| | | GGGTTAAGCCCCGCACTTTTAAGACCGACTTACGACGCCG |
| | | CCTGCGCGCCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACTTACGACGGGTAT |
| | | TGACCGTCGTACCATTCGTCCCGTATAACAGAACTTTACA |
| | | ACCCGAAGGCCGTCATCGTTCACGCGGCGTTGCTCCGTCA |
| | | GACTTTCGTCCATTGCGGAAGATTCCCCA |
| 22532 | 53 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTTC |
| | | CTCTCCGATACTCTAGCATCGCAGTTTCGGTCCCCTCACG |
| | | GGGTTAAGCCCCGCACTTTTAAGACCGACTTACGACGCCG |
| | | CCTGCGCGCCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACTTACGACGGGTAT |
| | | TGACCGTCGTACCATTCGTCCCGTATAACAGAACTTTACA |
| | | ACCCGAAGGCCGTCATCGTTCACGCGGCGTTGCTCCGTCA |
| | | GACTTTCGTCCATTGCGGAAGATTCCCCA |
| 121 | 54 | CCCGTTTGCTCCCCTGGCTTTCGCGCCTCAGCGTCAGTTG |
| | | TCGTCCAGAAAGCCGCTTTCGCCACTGGTGTTCCTCCTAA |
| | | TATCTACGCATTTCACCGCTACACTAGGAATTCCGCTTTC |
| | | CTCTCCGATACTCTAGCATCGCAGTTTCGGTCCCCTCACG |
| | | GGGTTAAGCCCCGCACTTTTAAGACCGACTTACGACGCCG |
| | | CCTGCGCGCCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACTTACGACGGGTAT |
| | | TGACCGTCGTACCATTCGTCCCGTATAACAGAACTTTACA |
| | | ACCCGAAGGCCGTCATCCCGCACGCGGCGTCGCTGCATCA |
| | | GGCTTGCGCCCATTGTGCAATATTCCCCA |
| 13007 | 55 | CCCGTTTGCTCCCCTGGCTTTCGCGCCTCAGCGTCAGTTG |
| | | TCGTCCAGAAAGCCGCTTTCGCCACTGGTGTTCCTCCTAA |
| | | TATCTACGCATTTCACCGCTACACTAGGAATTCCGCTTTC |
| | | CTCTCCGATACTCTAGCATCGCAGTTTCGGTCCCCTCACG |
| | | GGGTTAAGCCCCGCACTTTTAAGACCGACTTACGACGCCG |
| | | CCTGCGCGCCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACTCGTAACGGGTAT |
| | | TGACCGCTACGCCATTCGTCCCGTATAACAGAACTTTACA |
| | | ACCCGAAGGCCGTCATCGTTCACGCGGCGTTGCTCCGTCA |
| | | GACTTTCGTCCATTGCGGAAGATTCCCCA |
| 11273 | 56 | CCTGTTTGCTCCCCACGCTTTCGAGCCTCAACGTCAGTTA |
| | | CCGTCCAGTAAGCCGCCTTCGCCACTGGTGTTCCTCCTAA |
| | | TATCTACGCATTTCACCGCTACACTAGGAATTCCGCTTTC |
| | | CTCTCCGATACTCTAGCATCGCAGTTTCGGTCCCCTCACG |
| | | GGGTTAAGCCCCGCACTTTTAAGACCGACTTACGACGCCG |
| | | CCTGCGCGCCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACTCGTAACGGGTAT |
| | | TGACCGCTACGCCATTCGTCCCGTATAACAGAACTTTACA |
| | | ACCCGAAGGCCGTCATCGTTCACGCGGCGTTGCTCCGTCA |
| | | GACTTTCGTCCATTGCGGAAGATTCCCCA |
| 23919 | 57 | CCCGTTCGCTCCCCTGGCTTTCGTGCCTCAGCGTCAGTTG |
| | | TCGTCCAGAAAGCCGCTTTCGCCACTGGTATTCCTCCTAA |
| | | TATCTACGCATTTCACCGCTACACTAGGAATTCTGCTTTC |
| | | CTCTCCGATACTCCAGTCTCCCAGTTTCCGTCCCCTCACG |
| | | GGGTTGAGCCCCGCACTTTTAAGACAGACTTAAGAGACCG |
| | | CCTGCGCACCCTTTACGCCCAATAATTCCGGACAACGCTT |
| | | GCCACCTACGTATTACCGCGGCTGCTGGCACGTAGTTAGC |
| | | CGTGGCTTTCTCTTACGGTACCGTCACAACAGGATGGTAT |
| | | TAGCATCCTGCCCGTTCGTCCCGTATAACAGAGCTTTACA |
| | | ACCCGAAGGCCGTCTTCACTCACGCGGCGTTGCTCCGTCA |
| | | GACTTTCGTCCATTGCGGAAGATTCCCCA |
| 12611 | 58 | CCTGTTTGCTCCCCACGCTTTCGTGCCTCAGCGTCAGTTA |
| | | CAGTCCAGAGAGCCGCCTTCGCAACTGGTGTTCCTCCTAA |
| | | TATCTACGCATTTCACCGCTACACTAGGAATTCCACTCTC |
| | | CTCTCCTGCACTCAAGTCTTACAGTTTCAAAAGCTTACTA |
| | | CGGTTGAGCCGTAGCCTTTCACTTCTGACTTGAAAGACCG |
| | | CCTACGCACCCTTTACGCCCAGTAATTCCGGATAACGCTA |
| | | GCCCCCTACGTATTACCGCGGCTGCTGGCACGTAGTTAGC |
| | | CGGGGCTTCCTCCTCAAGTACCGTCATTATCTTCCTTGAG |
| | | GACAGAGCTTTACGACCCGAAGGCCTTCATCGCTCACGCG |
| | | GCGTTGCTGCATCAGGCTTTCGCCCATTGTGCAATATTCC |
| | | CCA |
| 14846 | 59 | CCTGTTTGCTCCCCACGCTTTCGAGCCTCAGCGTCAGTCA |
| | | TCGTCCAGTAAGCCGCCTTCGCCACTGGTGTTCCTCCTAA |
| | | TATCTACGCATTTCACCGCTACACTAGGAATTCCACTTAC |
| | | CCCTCCGACACTCTAGTCCGACAGTTTCCAATGCAGTACC |
| | | GGGGTTGAGCCCCGGGCTTTCACATCAGACTTGCCGTACC |
| | | GCCTGCGCTCCCTTTACACCCAGTAAATCCGGATAACGCT |
| | | TGCACCATACGTATTACCGCGGCTGCTGGCACGTATTTAG |
| | | CCGGTGCTTCTTAGTCAGGTACCGTCATTTCTTCTTCCCT |
| | | GCTGATAGAGCTTTACATACCGAAATACTTCTTCGCTCAC |
| | | GCGGCGTCGCTGCATCAGGGTTTCCCCCATTGTGCAATAT |
| | | TCCCCA |
| 25776 | 60 | CCTGTTTGCTACCCATGCTTTCGAGCCTCAGCGTCAGTTA |
| | | GTGCCCAGTAGGCCGCCTTCGCCACTGGTGTTCCTCCCGA |
| | | TATCTACGCATTCCACCGCTACACCGGGAATTCCGCCTAC |
| | | CTCTACACCACTCAAGACTGACAGTTTTGAAAGCAATTTA |
| | | TGGGTTGAGCCCATAGATTTCACTCCCAACTTGCCAATCC |
| | | GCCTGCGCTCCCTTTACACCCAGTAATTCCGGACAACGCT |
| | | TGCACCCTACGTTTTACCGCGGCTGCTGGCACGTAGTTAG |
| | | CCGGTGCTTCCTCCCAGGGTACCGTCATTATCGTCCCCTG |
| | | GGACAGGAGTTTACAATCCGAAGACCTTCTTCCTCCACGC |
| | | GGCGTCGCTGCATCAGGGTTTCCCCCATTGTGCAATATTC |
| | | CCCA |
| 13779 | 61 | CCTGTTTGCTCCCCACGCTTTCGCGCCTCACCGTCAGTTG |
| | | TCGTCCAGTAATCCGCCTTCGCCACTGGTGTTCTTCCTTA |
| | | TATCTACGCATTTCACCGCTACACAAGGAATTCCGATTAC |
| | | CTCTCCGATACTCAAGACCAACAGTTTCAAATGCAGTTCA |
| | | CGGGTTAAGCCCGTGGATTTCACATCTGACTTGCCGGCCC |
| | | GGCTGCACGCCCTTTACACCCAGTAAATCCGGACAACGCT |
| | | TGCCACCTACGTATTACCGCGGCTGCTGGCACGTAGTTAG |
| | | CCGTGGCTTATTCGTCAGGTACCGTCTTCTGCTCTTCCCT |
| | | GACAAAAGAAGTTTACAACCCGAAAGCCTTCTTCCTTCAC |
| | | GCGGCGTTGCTGGGTCAGACTTGCGTCCATTGCCCAATAT |
| | | TCCCCA |
| 24121 | 62 | CCTGTTTGCTCCCCACGCTTTCGAGCCTCAGCGTCAGTTA |
| | | CAGTCCAGAGAATCGCCTTCGCCACTGGTGTTCTTCCTAA |
| | | TCTCTACGCATTTCACCGCTACACTAGGAATTCCATTCTC |
| | | CTCTCCTGCACTCTAGATATCCAGTTTGGAATGCAGCTCC |
| | | CAAGTTAAGCTCGGGTATTTCACATCCCACTTAAATATCC |
| | | GCCTACGCTCCCTTTACGCCCAGTAAATCCGGATAACGCT |
| | | TGCCACCTACGTATTACCGCGGCTGCTGGCACGTAGTTAG |
| | | CCGTGGCTTCCTCCTTAGGTACCGTCATTATCGTCCCTAA |
| | | AGACAGAGCTTTACAATCCGAAGACCTTCATCACTCACGC |
| | | GGCGTTGCTGCATCAGGGTTTCCCCCATTGTGCAATATTC |
| | | CCCA |
| 25250 | 63 | CCTGTTTGCTCCCCACGCTTTCGAGCCTCAACGTCAGTCA |
| | | CCGTCCAGTAAGCCGCCTTCGCCACTGGTGTTCCTCCTAA |
| | | TATCTACGCATTTCACCGCTACACTAGGAATTCCGCTTAC |
| | | CTCTCCGGCACTCCAGAATTGCAGTTTCCAAAGCAGTCCC |
| | | AGGGTTGAGCCCTGGGTTTTCACTCCAGACTTGCCTCTCC |
| | | GTCTACGCTCCCTTTACACCCAGTAAATCCGGATAACGCT |
| | | TGCACCATACGTATTACCGCGGCTGCTGGCACGTATTTAG |
| | | CCGGTGCTTCTTAGTCAGGTACCGTCAGTTTCTTCCCTGC |
| | | TGATAGAAGTTTACATACCGAAATACTTCTTCCTTCACGC |
| | | GGCGTCGCTGCATCAGGGTTTCCCCCATTGTGCAATATTC |
| | | CCCA |
| 15039 | 64 | CCTGTTTGCTACCCATGCTTTCGAGCCTCAGCGTCAGTTG |
| | | GTGCCCAGTAGGCCGCCTTCGCCACTGGTGTTCCTCCCGA |
| | | TATCTACGCATTCCACCGCTACACCGGGAATTCCGCCTAC |
| | | CTCTGCACTACTCAAGGCCAGCAGTTTTGAAAGCAATTTA |
| | | TGGGTTGAGCCCATAGATTTCACTTCCAACTTGCCGGTCC |
| | | GCCTGCGCTCCCTTTACACCCAGTAATTCCGGACAACGCT |
| | | TGCCACCTACGTTTTACCGCGGCTGCTGGCACGTAGTTAG |
| | | CCGTGGCTTTCTTGTACGGTACCGTCATTATCGTCCCGTA |
| | | CGACAGGAGTTTACAATCCGAAAACCTTCTTCCTCCACGC |
| | | GGCGTCGCTGCATCAGGGTTTCCCCCATTGTGCAATATCC |
| | | CCCA |
| 8753 | 65 | CCTGTTTGCTACCCATGCTTTCGAGCCTCAGCGTCAGTTG |
| | | GTGCCCAGTAGGTCGCCTTCGCCACTGGTGTTCCTCCCGA |
| | | TATCTACGCATTCCACCGCTACACCGGGAATTCCACCTAC |
| | | CTCTGCACTACTCAAGGCCAGCAGTTTTGAAAGCAATTTA |
| | | TGGGTTGAGCCCATAGATTTCACTCCCAACTTGCCGGTCC |
| | | GCCTGCGCTCCCTTTACACCCAGTAATTCCGGACAACGCT |
| | | TGCACCCTACGTTTTACCGCGGCTGCTGGCACGTAGTTAG |
| | | CCGGTGCTTTCTTGTTAGGTACCGTCAGATATTCGTCCCT |
| | | AACGACAGGAGTTTACAATCCGAAAACCTTCTTCCTCCAC |
| | | GCGGCGTCGCTGCATCAGGGTTTCCCCCATTGTGCAATAT |
| | | CCCCCA |
| 17134 | 66 | CCTGTTTGCTCCCCACGCTTTCGCGCCTCAGTGTCAGTTA |
| | | CAGACCAGGAAGCCGCCTTCGCCACTGGTGTTCCTCCATA |
| | | TCTCTACGCATTTCACCGCTACACATGGAATTCCACTTCC |
| | | CTCTTCTGCACTCAAGTCGACCAGTTTCCAATGACCCTCC |
| | | ACGGTTAAGCCGTGGGCTTTCACATCAGACTTAATCAACC |
| | | ACCTGCGCGCTCTTTACGCCCAATAATTCCGGATAACGCT |
| | | CGCCACCTACGTATTACCGCGGCTGCTGGCACGTAGTTAG |
| | | CCGTGGCTTTCTCATAAGGTACCGTCACACTCTAGCCATT |
| | | TCCTACTAAAGTCGTTCTTCCCTTATAACAGAATTTTACA |
| | | ACCCGAAGGCCTTCATCATTCACGCGGCGTTGCTCGGTCA |
| | | GGCTTTCGCCCATTGCCGAAGATTCCCTA |
| 8468 | 67 | CCTGTTTGCTCCCCACGCTTTCGCGCCTCAGCGTCAGTTA |
| | | ATGTCCAGCAGGCCGCCTTCGCCACTGGTGTTCCTCCCGA |
| | | TATCTACGCATTCCACCGCTACACCGGGAATTCCGCCTGC |
| | | CTCTCCATCACTCAAGAACTACAGTTTCAAATGCAGTCTA |
| | | TGGGTTGAGCCCATAGTTTTCACATCTGACTTGCAATCCC |
| | | GCCTGCTCGCCCTTTACACCCAGTAAATCCGGATAACGCT |
| | | TGCCACCTACGTATTACCGCGGCTGCTGGCACGTAGTTAG |
| | | CCGTGGCTTATTCTTACGGTACCGTCATTTGTTTCGTCCC |
| | | GTACAAAAGAAGTTTACAATCCGAAAACCTTCATCCTTCA |
| | | CGCGGCATTGCTGCATCAGGGTTTCCCCCATTGTGCAATA |
| | | TTCCCCA |
| 22933 | 68 | CCTGTTTGCTCCCCACGCTTTCGAGCCTCAACGTCAGTTA |
| | | CAGTCCAGTAAGCCGCCTTCGCCACTGGTGTTCCTCCTAA |
| | | TATCTACGCATTTCACCGCTACACTAGGAATTCCGCTTAC |
| | | CTCTCCTGCACTCTAGCTTAACAGTTTCCAATGCAGTCTT |
| | | GGGGTTGAGCCCCAAGTTTTCACATCAGACTTGCCATGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCTTCCTTCACGC |
| | | GGCGTCGCTGCATCAGGGTTTCCCCCATTGTGCAATATTC |
| | | CCCA |
| 4404 | 69 | CCTGTTTGCTCCCCACGCTTTCGAGCCTCAACGTCAGTCA |
| | | TCGTCCAGCAAGCCGCCTTCGCCACTGGTGTTCCTCCTAA |
| | | TATCTACGCATTTCACCGCTACACTAGGAATTCCACTTGC |
| | | CTCTCCGACACTCTAGCTCTACAGTTCCAAATGCAGTCCC |
| | | GGGGTTGAGCCCCGGGCTTTCACATCTGGCTTGCAATGCC |
| | | GTCTACGCTCCCTTTACACCCAGTAAATCCGGATAACGCT |
| | | TGCCCCCTACGTATTACCGCGGCTGCTGGCACGTAGTTAG |
| | | CCGGGGCTTCTTAGTCAGGTACCGTCATTTTCTTCCCTGC |
| | | TGATAGAAGTTTACATACCGAAATACTTCATCCTTCACGC |
| | | GGCGTCGCTGCATCAGGGTTTCCCCCATTGTGCAATATTC |
| | | CCCA |
| 22561 | 70 | CCTGTTTGCTCCCCACGCTTTCGCACCTCAGCGTCAGTAT |
| | | TTGCCCAGGAGACCGCCTTCGCCTCTGGTGTTCTTTCAGA |
| | | TATCTACGCATTCCACCGCTACACCTGAAATTCCATCTCC |
| | | CTCTGCAATACTCTAGAAAGGCAGTTTAGAATGACCAACC |
| | | CAGGTTGAGCCCGGGTATTTCACATCCTACTTACCTTTCC |
| | | GCCTACATGCCCTTTACGCCCAGTTATTCCGATTAACGCT |
| | | TGCACCCTCCGTATTACCGCGGCTGCTGGCACGGAGTTAG |
| | | CCGGTGCTTCTTCTGTGGGTAACGTCAGTGTTTCCTCCCC |
| | | ACTGAAAGTGCTTTACAACCCGAAGGCCTTCTTCACACAC |
| | | GCGGCATGGCTGCATCAGGGTTTCCCCCATTGTGCAATAT |
| | | TCCCTA |
| 5544 | 71 | CCTGTTTGCTCCCCACGCTTTCGCACCTCAGCGTCAGTAT |
| | | TTGCCCAGGAGACCGCCTTCGCCTCTGGTGTTCTTTCAGA |
| | | TATCTACGCATTCCACCGCTACACCTGAAATTCCATCTCC |
| | | CTCTGCAATACTCTAGAAAGGTAGTTCAAAATGACCAACC |
| | | CAGGTTGAGCCCAGGTCTTTCACATCTTGCTTGCCTTTCC |
| | | GCCTACATGCCCTTTACGCCCAGTTATTCCGATTAACGCT |
| | | TGCACCCTCCGTATTACCGCGGCTGCTGGCACGGAGTTAG |
| | | CCGGTGCTTCTTCTGTGGGTAACGTCAGTATTTCCTCCCC |
| | | ACTGAAAGTGCTTTACAACCCGAAGGCCTTCTTCACACAC |
| | | GCGGCATGGCTGCATCAGGGTTTCCCCCATTGTGCAATAT |
| | | TCCCTA |
| 157 | 72 | CCTGTTTGCTACCCACGCTTTCGCATATGAGCGTCAGTAT |
| | | TTGCCCAGGAGACTGCCTTCGCCTTTGGTGTTCTTCCAGA |
| | | TCTCTACGCATTCCACCGCTACACCTGGAATTCCGTCTCC |
| | | CCCTGCAATACTCTAGTCCCACAGTTTAGAGTGCAATTCC |
| | | GAGGTTGAGCCCCGGGCTTTCACACCCTACTTGCAGAACC |
| | | GCCTGCATGCCCTTTACGCCCAGTTATTCCGATTAACGCT |
| | | TGCACCCTCCGTATTACCGCGGCTGCTGGCACGGAGTTAG |
| | | CCGGTGCTTCTTCTGAGGGTAACGTCATTTTCCTCCCCTC |
| | | TGAAAGTGCTTTACAACCCGAAGGCCTTCTTCACACACGC |
| | | GGCATGGCTGCATCAGGGTTCCCCCCATTGTGCAATATTC |
| | | CCTA |
| 3131 | 73 | CCTGTTTGCTACCCACGCTTTCGCATATGAGCGTCAGTAT |
| | | TTGCCCAGGAGACTGCCTTCGCCTTTGGTGTTCTTCCAGA |
| | | TATCTACGCATTCCACCGCTACACCTGGAATTCCGTCTCC |
| | | CCCTGCAATACTCTAGTCCACCAGTTTAGTATGCTGTTCC |
| | | CAGGTTGAGCCCGGGGATTTCACATCCTACTTGATGAACC |
| | | GCCTGCATGCCCTTTACGCCCAGTTATTCCGATTAACGCT |
| | | TGCACCCTCCGTATTACCGCGGCTGCTGGCACGGAGTTAG |
| | | CCGGTGCTTCTTCTGAGGGTAACGTCATTTTCTTCCTCCC |
| | | CTCCGAAAGTGCTTTACAACCCGAAGGCCTTCTTCACACA |
| | | CGCGGCATGGCTGCATCAGAGTTTCCTCCATTGTGCAATA |
| | | TTCCCTA |

### Method

The method involves the extraction of DNA from a freshly produced faecal sample or sample from the gastrointestinal tract of a feline. Extraction of the DNA can be conducted by a means such as the QIAamp Power Faecal DNA kit (Qiagen) or similar and subsequently the use of molecular biology techniques to assess the detection rate and abundance of the bacterial taxa or DNA, RNA or protein sequences characteristic of those taxa described in FIG. 2 (Table 1.1) and Table 1.2 or alternative biomarkers for those organisms compared to standardised healthy control samples and to animals with chronic gastrointestinal enteropathy, IBD, acute diarrhoea and chronic 10 diarrhoea. The interpretation of health status is then made based on the combination and relative abundance of the health associated organisms detected in the faeces or gastrointestinal contents of the cat compared to control samples from cats of the same microbiome life stage and/or from the same individual over time to allow the assessment of health status of the microbiome in the individual and to indicate how the health of the microbiome can be enhanced.

Assessment of the microbiome components observed in the faeces or GI sample from the cat can be undertaken at an individual point in time for assessment against healthy and/or clinical controls in the same life stage, to receive a description of the relative health of the microbiome at a specific timepoint. Alternatively, the gastrointestinal health of the cat can be monitored over time by assessment of the gut microbiome periodically at intervals such as 6 monthly or one yearly tests/assessments or following particular events such as gastrointestinal upset, or travel. The results of detection and relative abundance of the microbial species associated with health (or with the disease condition) can then be compared with the previous results or cumulative (averaged) results from the previous assessments of the microbiome from the individual cat. In the case of longitudinal assessment in an individual over time, adjustments must be made as the animal crosses from one microbiome life stage to the next by additional comparisons to control cohorts such as provided within the data reported here (FIG. 3 (Table 1.3)).

After DNA extraction from freshly produced faeces and sequencing of the DNA by techniques such as 16S rDNA amplicon, shotgun, metagenome, Illumina, nanopore or other DNA sequencing techniques, the resulting DNA sequences are clustered to species (>98% ID) level. Assessment of the relative abundance of the sequences descriptive of the organisms in Table 1.1 or DNA sequences within 95% identical to those in Table 1.2 or other DNA, RNA or protein sequences or biomarkers characteristic of those species specified in FIG. 2 (Table 1.1) and Table 1.2 is made. Briefly, sequence data obtained from the test sample is clustered into groups of sequences with 98%-100% identity and a reference sequence from the clusters which represent >0.001% of the total sequences is then used to either 1) assign taxonomy or gene function through database homologues or to determine the nature of the biomarker through homology searches of DNA databases such as the Greengenes or Silva or the NCBI non-redundant nucleotide sequence database for comparison to known DNA sequences of species held within the databases or 2) compared to the DNA sequences given in Table 1.2.

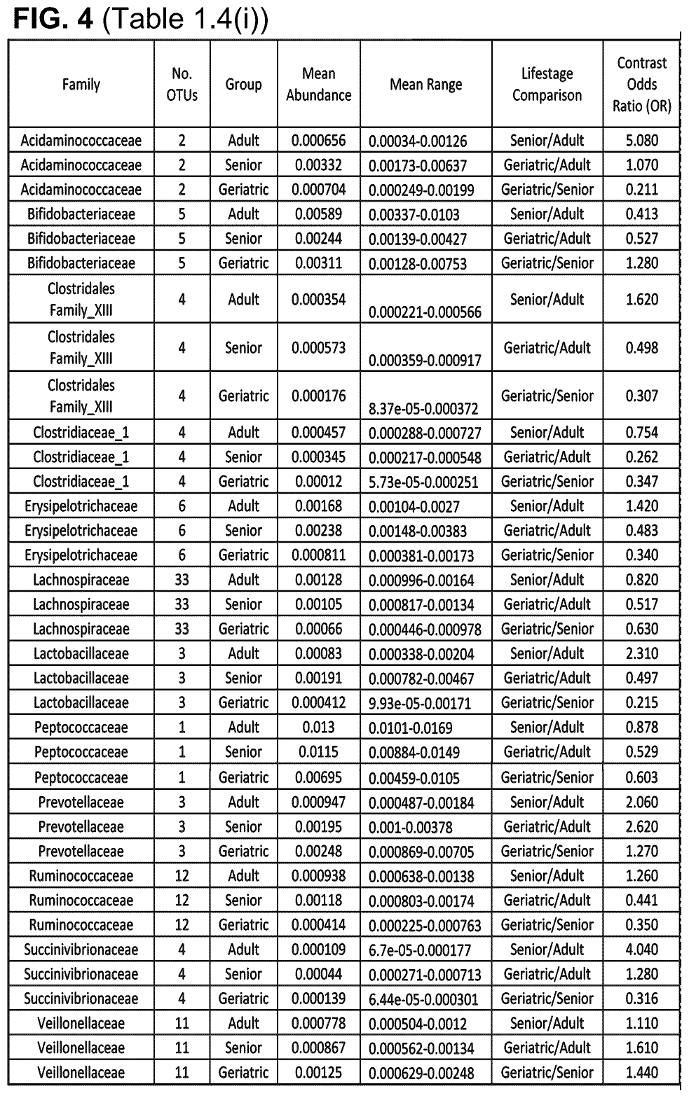

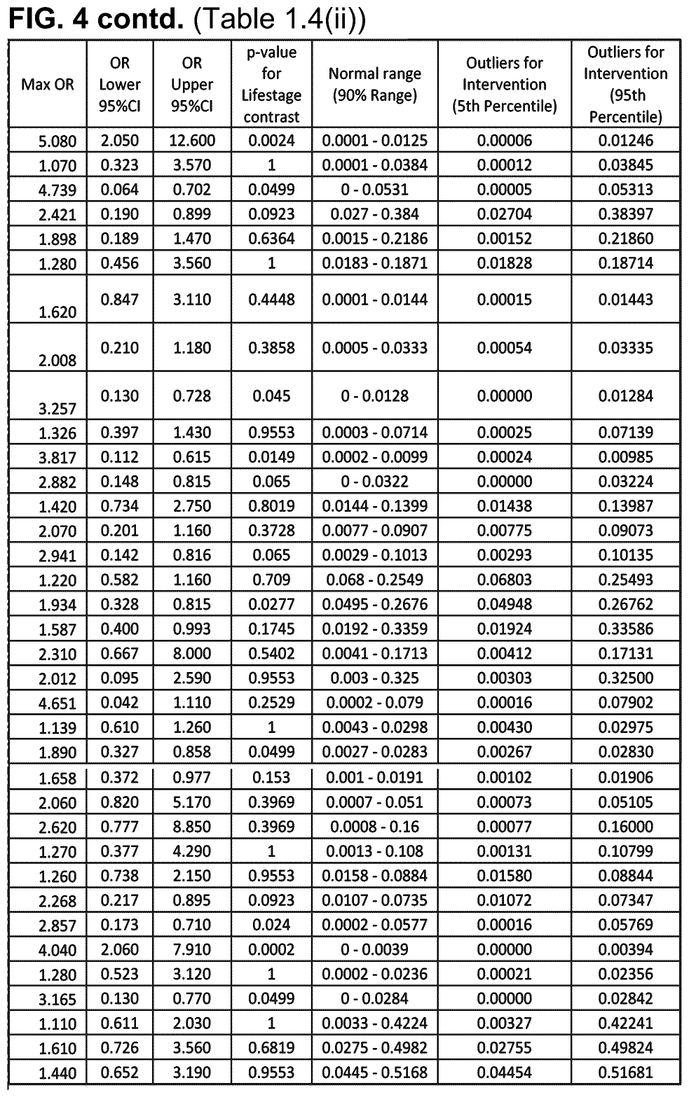

The number and abundance of the organisms, sequences or biomarkers described within FIG. 2 (Table 1.1) and Table 1.2 are then used in comparison of the test sample to controls for the number of organisms and abundance of the individual and total load of the health associated species described in FIG. 3 (Table 1.3). Should the bacterial content or abundance in the faeces or GI sample fall below the notification point listed for the organisms of all of the organisms from a genus this is indicative that the animal can benefit from an intervention to support that bacterial genus to through interventions such as dietary manipulation, supplementation, or other supportive means. Notification points for species and genera (by addition of the relevant species) suggesting corrective action are given in FIG. 3 (Table 1.3), while notification for intervention points for microbial families are given in FIG. 4 (Table 1.4).

FIG. 3 (Table 1.3) provides microbiome features as described by bacterial taxa for detection of gut microbiome health in cats throughout progressive life stages.

FIG. 4 (Table 1.4) provides bacterial families associated with health in mammals and with utility for the detection of health in cats.

### Example 2: a Method of Detecting Health in the Feline Gut Microbiome Based on the Microbial Diversity in Faeces

### Introduction to Bacterial Diversity in the Gut Microbiome and Health

Diversity in the bacterial faecal of the gastrointestinal microbiota in humans has been associated with race/ethnicity, nutritional status, dietary diversity and with host health [32;33]. In humans the infant faecal microbiota increases in diversity as it matures, with peaks in development at weaning and a plateau in diversity aligning with relative compositional stability similar to the adult-like state at around three years of age [34;35].

The early colonization and subsequent maturation, including the development of diversity in the gut microbiome is reported to have long-term health implications for the human host with possible implications on immune function and allergic disease incidence impacting health in later life. The relationships are however complex with C-section birth and formula feeding reported to effect diversity compared to natural modes of birth and breast feeding [36;32;37;38].

Analysis of the microbiome in 8-16 week old kittens that had been fully weaned previously at 5 weeks of age, observed the microbiota to be compositionally similar to that of adult felines showing stability in diversity over time [12]. Similarly, a longitudinal study by Bermingham et al. (2018) initiated in young cats (8-17 weeks of age) did not describe altered diversity with age post weaning. However, little is known about the developing microbiota in kittens pre-weaning and hence, research to investigate the developing microbiota in cats during early life prior to, and throughout weaning, would further the understanding of the microbiome, particularly where longer term health parameters can subsequently be followed.

At the other extreme of life stage in humans, diversity in the gut microbiota has been observed to decrease by several groups and this reduced diversity is reported to correlate with dietary diversity, frailty, the ability to perform tasks and altered markers of systemic immunity [40;41]. Despite compositional differences detected in the faecal microbiota in older cats compared to their younger counterparts, comparisons of diversity have not previously been reported. Although associations of diversity with health have been described in both cats and dogs and have been applied to the prediction of inflammatory bowel disease in dogs [42].

Research into human C. difficile-associated and C. difficile-negative nosocomial diarrhoca has demonstrated similar phenomena to that in pets [43], whereby the microbiota of the distal gut demonstrated reduced phylogenetic diversity and species richness in disease compared to clinical health [44]. Altered diversity in the human study was driven by changes in the abundance of organisms from the Phylum Firmicutes and particularly in the Ruminococcaceae spp., Lachnospiraceae spp. and butyrate producers.

Commonly identified changes in the microbiota of pets with inflammatory bowel disease (IBD) includes decreased abundance of Firmicutes and Bacteroidetes, and increased abundance of Proteobacteria. Within the Firmicutes Phylum reduced diversity of Clostridium clusters XIVa and IV (i.e., Lachnospiraceae and Clostridium coccoides subgroups) have also been described in IBD, suggesting that these bacterial groups can play an important role in maintenance of

gastrointestinal health [45]. Research has suggested that dietary characteristics such as macronutrient content can show potential for the management of diversity in the gut microbiota in cats. Kittens fed a high protein, low carbohydrate diet showed greater species richness and microbial diversity than those fed on a medium protein medium carbohydrate diet, with gross differences across 324 genera between diet groups [12]. In this study, diet was found to be a dominant force in influencing the taxonomy of the microbiota compared to age. Microbiome analysis described 2,013 putative enzyme function groups that were different between diet groups, six of which belonged to pathways associated with amino acid biosynthesis and metabolism thus suggesting the changes in the microbiome were linked to putative differences in protein metabolism.

To date, therefore insights on the diversity characteristics of the gut microbiota in cats align largely with the human and canine microbiome research. Further understanding of the development of diversity and compositional characteristics in kittens preweaning can serve to enhance the understanding of the longer term impacts of the gut microbiota on feline health, including the impact of microbial diversity. However, similarly to the human and canine hosts apparent opportunities exist to manipulate compositional and diversity of the gut microbiota in cats towards the enhancement of host heath and to utilize the life stage dependent characteristics of diversity to contribute in the assessments of the health status of the gastrointestinal microbiome in cats throughout life.

### Methods

Shannon diversity was calculated for each sample based on the OTU/taxon count and relative abundance according to the equation shown below. Shannon diversity was modelled using a linear mixed effects model with a fixed effect of age group and random intercept of pet. Pairwise comparisons of the life stage groups were performed with a controlled familywise error rate of 5%.

$$Shannon Index ( H ) = - {\sum }_{i = 1}^{s} {p}_{i} ln {p}_{i}$$

Equation used for the calculation of Shannon diversity from bacterial sequence clustering and abundance data

### Results

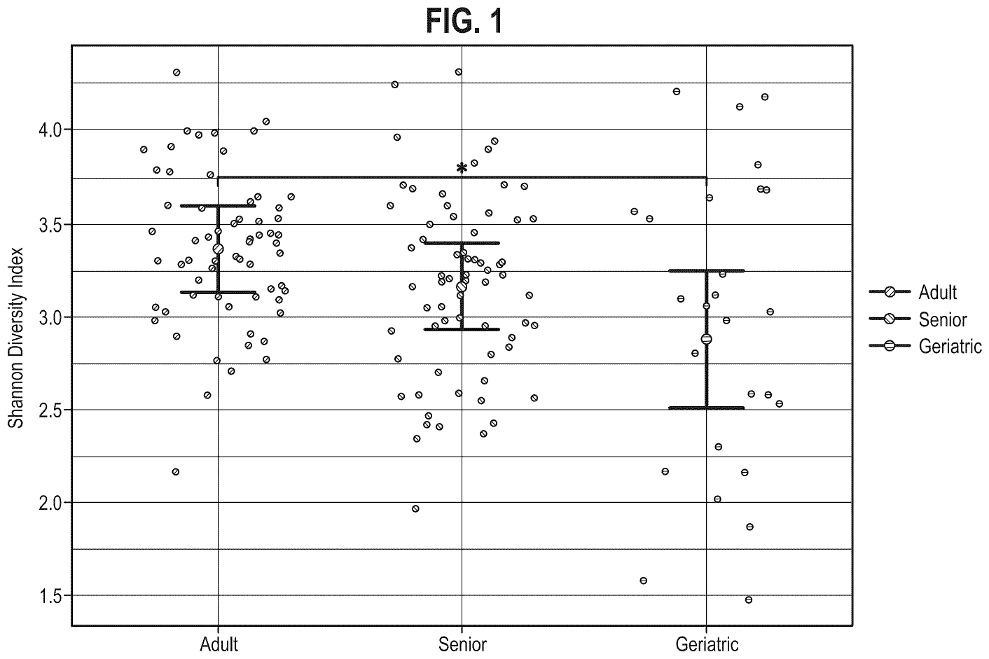

Assessment of Shannon diversity in the microbiota of cats from adult senior and geriatric cats yielded diversity estimates (FIG. 1 and Table 2.1). A statistically significant difference was observed in the Shannon diversity of the faecal microbiome of adult cats (mean age 5.66 years) compared to those in the geriatric population (mean age 14.78 years).

### Method

The method involves the extraction of DNA from a freshly produced faecal sample by a means such as the QIAamp Power Faecal DNA kit (Qiagen) and subsequently the use of molecular biology techniques to detect the 16S rDNA or rRNA present or other genetic features enabling determination of bacterial abundance and taxon or species richness of the microbial community in faeces or other gastrointestinal sample. After DNA/RNA extraction from freshly produced faeces and genetic sequence analysis by techniques such as 16S rRNA/16S rDNA amplicon, shotgun, metagenome, Illumina, nanopore or other sequencing technique, the resulting sequences are clustered to species (>98% ID) level and the relative abundance of each taxa is determined for the individual OTUs as a proportion of the total. The total number of sequences or OTUs and OTU relative abundance data are then used to calculate diversity which accounts for both abundance and evenness of the species detected. Any diversity calculation can be used such as Shannon diversity or other alpha diversity calculations or alternatively beta diversity can be used. Shannon Diversity can be calculated by the following method:

$$Shannon Index ( H ) = - {\sum }_{i = 1}^{s} {p}_{i} ln {p}_{i}$$

After the determination of diversity of the microbiome for the test sample using functions such as Shannon diversity indices or other alpha or beta diversity assessment (including total OTU number with the sample) diversity can be compared to standardised samples from healthy control populations within the same life stage (see Table 2.1) and to animals of similar age with chronic gastrointestinal enteropathy, IBD, acute or chronic diarrhoea or other gastrointestinal symptoms.

The interpretation of microbiome health is based on the level of the diversity detected in the faeces of the cat in context of the animal's life stage compared to the healthy control samples and samples from animals with gastrointestinal conditions including IBD, gastrointestinal enteropathy or chronic and acute diarrhoea or other GI condition. Interpretation can also include comparison to previous analyses of the same cat at different timepoints either from stored samples or using previously collected data. In the case of assessment within the individual over time the gastrointestinal health of the cat can be monitored over time by testing/assessment of the gut microbiome periodically at intervals such as 6 monthly or annual or following particular events such as gastrointestinal upset, or travel. The results of assessment of the microbial diversity can then be compared with the previous results or cumulative (averaged) results from the previous assessments of the microbiome from the individual cat.

The interpretation of the health status of the gut microbiome in the sample is then made based on the level of the diversity detected in the faeces of the cat in context of the animal's life stage (kitten, adult, senior or geriatric life stage) to allow the assessment of microbiome health according to the parameters described in Table 2.1. Outliers beyond the 5th and 95th percentile of the population range can be candidates for targeted enhancement of the gut microbiome through dietary or other means to alter the faecal microbiome or microbiota diversity towards the 90 percentile population range. The direction and magnitude of change in the gut microbial diversity 10 can be interpreted from those described in Table 2.1.

TABLE 2.1
Estimated Shannon index diversity expressed as group means with
95% confidence intervals in adult, senior and geriatric cats.

| | | | | | | Outliers for | |
|---|---|---|---|---|---|---|---|
| | | | | | | intervention | |
| | | | | Mean range | | 5th | 95th |
| Life | Age (years) | | Mean | 95% | 95% | Percentile | Percentile |
| stage | Mean | Range | Diversity | Lower CI | Upper CI | of range | of range |
| Adult | 5.66 | 4.7-6.8 | 3.37 | 3.14 | 3.60 | 2.77 | 3.99 |
| Senior | 10.1 | 8.1-12.5 | 3.16 | 2.93 | 3.40 | 2.41 | 3.92 |
| Geriatric | 14.78 | 12.6-16.2 | 2.88 | 2.51 | 3.25 | 1.65 | 4.17 |

| | | | | | |
|---|---|---|---|---|---|
| Mean diversity | 3.14 | 2.86 | 3.42 | 2.28 | 4.03 |

### REFERENCES

[1] Frank et al. (2007) Proc. Natl. Acad. Sci. USA 104, 13780-13785.
[2] Gevers et al. (2014) Cell Host Microbe 15, 382-392.
[3] Ni et al. (2017) Sci. Transl. Med. 9, eaah6888.
[4] Kostic et al. (2013) Cell Host Microbe 14, 207-215.
[5] Johnson and Foster (2018) Nature Reviews Microbiology, Oct; 16 (10): 647-655
[6] Kirchoff et al. (2018) Peer J Preprints 6: e26990v1
[7] Handl, S., et al., FEMS Microbiol Ecol, 2011. 76 (2): p. 301-10.
[8] Ley, et al. Science, 320 (5883), pp. 1647-1651.
[9] Marks, et al. Journal of Veterinary Internal Medicine, 25 (6), pp. 1195-1208.
[10] Minamoto et al.; Vet Microbiol 174 (3-4): 463-473.
[11] Suchodolski, J. S., 2016. The Veterinary Journal, 215, pp. 30-37.
[12] Deusch, O., et al., PLOS One, 2014. 9 (7): p. e101021.
[13] Laflamme, D. and Gunn-Moore, D., 2014. Veterinary Clinics: Small Animal Practice, 44 (4), pp. 761-774.
[14] Kuzmuk, et al., The Journal of nutrition, 135 (8), pp. 1940-1945.
[15] Woudstra, T. and Thomson, A. B., 2002. Best Practice & Research Clinical Gastroenterology, 16 (1), pp. 1-15.
[16] Suchodolski et al. 2015. PLOS ONE DOI: 10.137
[17] Inness et al. J Anim Physiol Anim Naar (Ber). 2007 February;91 (1-2): 48-53.
[18] Moon et al. Microbiology Open. 2018; 7e677
[19] Ritchie et al.; J Vet Int Med, 2008, vol 22, p803 abstract #334
[20] Hart et al. (2015) PLOS One. November 24; 10 (11): e0143334
[21] WO2018/006080
[22] Gennaro (2000) Remington: The Science and Practice of Pharmacy. 20th edition, ISBN: 0683306472.
[23] Molecular Biology Techniques: An Intensive Laboratory Course, (Ream et al., eds., 1998, Academic Press).
[24] Methods In Enzymology (S. Colowick and N. Kaplan, eds., Academic Press, Inc.)
[25] Handbook of Experimental Immunology, Vols. I-IV (D. M. Weir and C. C. Blackwell, eds, 1986, Blackwell Scientific Publications)
[26] Sambrook et al. (2001) Molecular Cloning: A Laboratory Manual, 3rd edition (Cold Spring Harbor Laboratory Press).
[27] Handbook of Surface and Colloidal Chemistry (Birdi, K. S. ed., CRC Press, 1997)
[28] Ausubel et al. (eds) (2002) Short protocols in molecular biology, 5th edition (Current Protocols).
[29] PCR (Introduction to Biotechniques Series), 2nd ed. (Newton & Graham eds., 1997, Springer Verlag)
[30] Current Protocols in Molecular Biology (F. M. Ausubel et al., eds., 1987) Supplement 30
[31] Smith & Waterman (1981) Adv. Appl. Math. 2:482-489.
[32] Sordillo et al.; 2017. Exercise and associated dietary extremes impact on gut microbial diversity. Gut, pp. gutjnl-2013.
[33] Clarke et al.; 2014. Journal of Allergy and Clinical Immunology, 139 (2), pp. 482-491.
[34] Yatsunenko, T., et al., Human gut microbiome viewed across age and geography. Nature, 2012. 486 (7402): p. 222-7
[35] Dominguez-Bello, M. G., et al., Proc Natl Acad Sci USA, 2010. 107 (26): p. 11971-5.
[36] Jakobsson et al. 2014. Gut, 63 (4), pp. 559-566.
[37] Abrahamsson et al.; 2014. Clinical & Experimental Allergy, 44 (6), pp. 842-850.
[38] Mueller et al.; 2015. Trends in molecular medicine, 21 (2), pp. 109-117.
[39] Bermingham et al., 2018. Frontiers in microbiology, 9.
[40] Claesson et al.; Nature. 2012 Aug. 9;488 (7410): 178-84.
[41] Biagi et al.; PLOS One. 2010; 5 (5): e10667.
[42] Vázquez-Baeza et al., 2016. Nature microbiology, 1 (12), p. 16177.
[43] Guard et al., PLOS One, 2017. 12 (4): p. e0175718.
[44] Antharam et al., 2013 Journal of clinical microbiology, pp. JCM-00845.
[45] Honneffer et al; World J Gastroenterol. 2014 Nov. 28; 20 (44): 16489-16497

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of treating an unhealthy microbiome in a feline in need thereof, the method comprising administering to the feline an effective amount of a functional food, a supplement, a nutraceutical, a live biotherapeutic product, a pharmaceutical composition, or combination thereof;
wherein a fecal sample or a sample from a gastrointestinal lumen from the feline comprises relative abundances of at least four bacterial species that fall outside a range of the relative abundances of said species in a control data set.

2. The method of claim 1, wherein the bacterial species are from genera or families selected from the group consisting of [Eubacterium], [Eubacterium] hallii group, Anaerobiospirillum, Anaerostipes, Anaerotruncus, Bifidobacterium, Blautia, Butyricicoccus, Catenibacterium, Clostridium sensu stricto 1, Collinsella, Coriobacteriaceae, Faecalibacterium, Holdemanella, Lachnoclostridium, Lachnospiraceae, Lachnospiraceae [Eubacterium] hallii group, Lachnospiraceae [Ruminococcus] gauvreauii, Lachnospiraceae FCS020 group, Lachnospiraceae genus, Lachnospiraceae NK4A136 group, Lactobacillus, Megamonas, Megasphaera, Peptoclostridium [Clostridium], Romboutsia, Roseburia, Ruminococcaceae, Ruminococcaceae UCG-009, Sarcina, Sellimonas, Subdoligranulum, Succinivibrio, and Turicibacter.

3. The method of claim 2, wherein the bacterial species are selected from the group consisting of [Clostridium] hiranonis, [Eubacterium] brachy, [Eubacterium] hallii group sp., Anaerobiospirillum succiniciproducens, Anaerostipes sp., Anaerotruncus sp., Bifidobacterium sp., Bifidobacterium saeculare, Blautia [Ruminococcus] gnavus, Blautia sp., Butyricicoccus sp., Catenibacterium sp., Clostridium sensu stricto 1 sp., Collinsella sp., Coriobacteriaceae UCG-002/UCG-003, Faecalibacterium sp., Holdemanella sp., Lachnoclostridium sp., Lachnospiraceae sp., Lachnospiraceae [Eubacterium] hallii group sp., Lachnospiraceae [Ruminococcus] gauvreauii group sp., Lachnospiraceae [Ruminococcus] gauvreauii group sp., Lachnospiraceae FCS020 group sp., Lachnospiraceae genus sp., Lachnospiraceae NK4A136 group sp., Lactobacillus sp., Megamonas sp., Megasphaera sp., Romboutsia sp., Roseburia sp., Ruminococcaceae sp., Sarcina sp., Sellimonas sp., Subdoligranulum sp., Succinivibrio sp., Turicibacter sp., and UCG-009 sp.

4. The method of claim 1, wherein the bacterial species have a 16S rDNA sequence with at least 95% identity to the sequence of any one of SEQ ID Nos: 3-73.

5. The method of claim 1, wherein the control data set comprises microbiome data of a feline at the same life stage.

6. A method of treating an unhealthy microbiome in a feline in need thereof, the method comprising administering to the feline an effective amount of a functional food, a supplement, a nutraceutical, a live biotherapeutic product, a pharmaceutical composition, or a combination thereof;
wherein a fecal sample or a sample from the gastrointestinal lumen from the feline comprises a Shannon diversity index that falls outside a healthy range.

7. The method of claim 6, wherein the feline is an adult and the healthy range is from about 2.0 to about 4.5.

8. The method of claim 6, wherein the feline is a senior and the healthy range is from about 2.41 to about 3.92.

9. The method of claim 6, wherein the feline is geriatric and the healthy range is from about 1.65 to about 4.17.

10. The method of claim 1, wherein the treatment brings the relative abundances of the at least four bacterial species in the sample to a healthy range.

11. The method of claim 1, wherein the unhealthy microbiome is associated with dysbiosis, irritable bowel syndrome, ulcerative colitis, Crohn's disease, or inflammatory bowel disease.

12. The method of claim 6, wherein the unhealthy microbiome is associated with dysbiosis, irritable bowel syndrome, ulcerative colitis, Crohn's disease, or inflammatory bowel disease.

13. The method of claim 6, wherein the treatment brings the Shannon diversity to a healthy range.

14. A method of monitoring the microbiome health in a feline who has undergone a diet change and/or has received a supplement or functional food or a pharmaceutical or nutraceutical composition which is able to change the microbiome composition, comprising determining the health of the microbiome by quantitating four or more bacterial species in a sample obtained from the feline to determine their relative abundance; and comparing the relative abundance to the relative abundance of the same species in a control data set; wherein an increase or decrease in the abundance of the four or more bacterial species relative to the control data set is indicative of an unhealthy microbiome.

15. The method of claim 14, wherein the health of the microbiome is determined before and after diet change and/or administration of the supplement or functional food or pharmaceutical or nutraceutical composition.

16. The method of claim 14, wherein the supplement or functional food or nutraceutical composition or pharmaceutical composition comprises bacteria.


SEQUENCE LISTING (73 sequences)

SEQ ID NO: 1 - DNA, 58 bp, synthetic construct
  misc_feature 1..58: note = Description of Artificial Sequence: Synthetic primer
  source 1..58: mol_type = other DNA
caagcagaagacggcatacgagatgtgactggagttcagacgtgtgctcttccgatct

SEQ ID NO: 2 - DNA, 58 bp, synthetic construct
  misc_feature 1..58: note = Description of Artificial Sequence: Synthetic primer
  source 1..58: mol_type = other DNA
aatgatacggcgaccaccgagatctacactctttccctacacgacgctcttccgatct

SEQ ID NO: 3 - DNA, 409 bp, Bifidobacterium sp.
  source 1..409: mol_type = other DNA
cctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcaacgggtaaactcactctcgcttgctccccgataaaagaggtttacaacccgaaggcctccatccctcacgcggcgtcgctgcatcaggcttgcgcccattgtgcaatattcccca

SEQ ID NO: 4 - DNA, 413 bp, Bifidobacterium sp.
  source 1..413: mol_type = other DNA
cctgttcgctccccacgctttcgctcctcagcgtcagtgacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcaccccgaagggcttgctcccagtcaaaagcggtttacaacccgaaggccgtcatcccgcacgcggcgtcgctgcatcaggcttccgcccattgtgcaagattcccca

SEQ ID NO: 5 - DNA, 411 bp, Bifidobacterium sp.
  source 1..411: mol_type = other DNA
cctgttcgctccccacgctttcgctcctcagcgtcagtaacggcccagagacctgccttcgccattggtgttcttcccgatatctacacattccaccgttacaccgggaattccagtctcccctaccgcactccagcccgcccgtacccggcgcagatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcacccgaaggcttgctcccgatcaaaagcggtttacaacccgaaggccgtcatcccgcacgcggcgtcgctgcatcaggcttgcgcccattgtgcaatattcccca

SEQ ID NO: 6 - DNA, 411 bp, Bifidobacterium sp.
  source 1..411: mol_type = other DNA
cctgttcgctccccacgctttcgctcctcagcgtcagtaacggcccagagacctgccttcgccattggtgttcttcccgatatctacacattccaccgttacaccgggaattccagtctcccctaccgcactccagcccgcccgtacccggcgcagatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcacccgaaggcttgctcccgatcaaaagcggtttacaacccgaaggcctccgtcccgcacgcggcgtcgctgcgtcagggttccccccattgcgcaagattcccca

SEQ ID NO: 7 - DNA, 409 bp, Bifidobacterium saeculare
  source 1..409: mol_type = other DNA
cctgttcgctccccacgctttcgctcctcagcgtcagtaacggcccagagacctgccttcgccattggtgttcttcccgatatctacacattccaccgttacaccgggaattccagtctcccctgccgcactccagcccgcccgtacccggcgcagatccaccgttaagcgatggactttcacaccagacgcgacgaaccgcctacgagccctttacgcccaataaatccggataacgcttgcaccctacgtattaccgcggctgctggcacgtagttagccggtgcttattcaacgggtacactcactctcgcttgctccccgataaaagcggtttacaacccgaaggcctccatcccgcacgcggcgtcgctgcgtcaggctttcgcccattgcgcaatattcccca

SEQ ID NO: 8 - DNA, 404 bp, Collinsella sp.
  source 1..404: mol_type = other DNA
cctgttcgctcccccagctttcgcgcctcagcgtcggtcacggcccagagggccgccttcgccaccggtgttccacccgatatctgcgcattccaccgctacaccgggtgttccaccctcccctaccggacccaagcccggaggttccgggggcgggcgggggttgagcccccgcatttgacccccggcctgccaggccgcctacgcgcgctttacgcccaatgaatccggataacgctcgccccctacgtattaccgcggctgctggcacgtagttagccggggcttcttctgcaggtacagtcttgtctcttccctgctgaaagcggtttacgacccgaaggcctccgtcccgcacgcggcgtcgctgcgtcagggttgcccccattgcgcaagattcccca

SEQ ID NO: 9 - DNA, 404 bp, Collinsella sp.
  source 1..404: mol_type = other DNA
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtacagtcttgtctcttccctgctgaaagcggtttacgacccgaaggcctccatcccgcacgcggcgtcgctgcgtcagggttgcccccattgcgcaagattcccca

SEQ ID NO: 10 - DNA, 404 bp, Collinsella sp.
  source 1..404: mol_type = other DNA
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcgggtaccgtcttgactcgtccccgctgaaagcggtttacgacccgaaggccttcgtcccgcacgcggcgtcgctgcgtcagggtttcccccattgcgcaatattcccca

SEQ ID NO: 11 - DNA, 405 bp, Collinsella sp.
  source 1..405: mol_type = other DNA
cctgttcgctcccccagctttcgcgcctcagcgtcggtctcggcccagagggccgccttcgccaccggtgttccacccgatatctgcgcattccaccgctacaccgggtgttccaccctcccctaccggacccaagccgcggaggttccgggggcttcggggggttgagccccccgcttcgacccccggcctgccgggccgcctacgcgcgctttacgcccaatgaatccggataacgctcgccccctacgtattaccgcggctgctggcacgtagttagccggggcttcttctgcaggtacagtcttgactcttccctgctgaaagcggtttacgacccgaaggcctccgtcccgcacgcggcgtcgctgcgtcagggttccccccattgcgcaagattcccca

SEQ ID NO: 12 - DNA, 404 bp, Collinsella sp.
  source 1..404: mol_type = other DNA
cctgttcgctcccccagctttcgcgcctcagcgtcggtctcggcccagagggccgccttcgccaccggtgttccacccgatatctgcgcattccaccgctacaccgggtgttccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtaccgtcttgcctcatccctgctgaaagcggtttacgacccgagggccttcgtcccgcacgcggcgtcgctgcgtcagggttccccccattgcgcaagattcccca

SEQ ID NO: 13 - DNA, 408 bp, unidentified
  misc_feature 1..408: note = Description of Unknown: Coriobacteriaceae family UCG-002/UCG-003 bacterium sequence
  source 1..408: mol_type = other DNA
cctgttcgctcccccagctttcgctcctcagcgtcagtgatggcccagaaagccgccttcgccaccggtgttcttcccgatatctgcgcattccaccgctacaccgggaattccgctttcccctaccacactcaagtccaccggtatcgggggcggaccggggttgagccccgggatttgacctccgacctaatggaccgcctacgagcgctttacgcccaatgaatccggataacgctcgccccctacgtattaccgcggctgctggcacgtagttagccggggcttcttctgcaggtaccgtcactttcgcctcgtccctgctgaaagcggtttacgacccgaaggccttcatcccgcacgcggcgtcgctgcgtcaggctttcgcccattgcgcaagattcccca

SEQ ID NO: 14 - DNA, 403 bp, Clostridium hiranonis
  source 1..403: mol_type = other DNA
cctgtttgctccccacgctttcgtgcctcagcgtcagttacagtccagaaagccgccttcgctactggtgttcctcccaatatctacgcatttcaccgctacactgggaattccgctttcctctcctgcactcaagtcagacagtatcaggagcttactacggttgagccgtagcctttaactcctgacttgaaagaccgcctacgcaccctttacgcccagtaaatccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggcttgcgcccattgtgcaatattcccca

SEQ ID NO: 15 - DNA, 404 bp, Eubacterium sp.
  source 1..404: mol_type = other DNA
cctgtttgctccccacgctttcgtgcctcagcgtcagttccagtccagcaggccgccttcgccactggtgttcctcctaatatctacgcatttcaccgctacactaggaattccgcctgcctctcctggactccagcaaaacagtttcaaatgcagccccggagttgagccccggtctttcacatctgacttgccttgccgcctacgcaccctttacacccagtaaatccggataacgcttgctccatacgtattaccgcggctgctggcacgtatttagccggagcttcttagtcaggtaccgtcattatcttccctgctgatagagctttacataccgaaatacttcttcactcacgcggcgttgctgcatcagggtttcccccattgtgcaatattcccca

SEQ ID NO: 16 - DNA, 406 bp, Eubacterium brachy
  source 1..406: mol_type = other DNA
cctgtttgctccccacgctttcgtacctcagtgtcagttacagtccagaaagccgccttcgccactggtgttcctcctaatatctacgcatttcaccgctacactaggaattccgctttcctctcctgcactcaagtttgccagttcgcagggcgaacaatggttgagccattgccttaaaccctgcgcttggtaaaccacctacgtaccctttacgcccaataattccggataacgcttgccccctccgtattaccgcggctgctggcacggagttagccggggcttcctccaagggtaccgtcatttgtttcttccccaaggacagagctttacgacccaaaggccttcatcgctcacgcggcgttgctgcatcaggcttgcgcccattgtgcaatattcccca

SEQ ID NO: 17 - DNA, 404 bp, Anaerostipes sp.
  source 1..404: mol_type = other DNA
cctgtttgctccccacgctttcgtgcctcagtgtcagtttcagtccagtaagccgccttcgccactgatgttcctcctaatatctacgcatttcaccgctacactaggaattccgcttacctctcctgcactccagttcgacagtttcaaaagcagtcccgcggttaagccccgggctttcacttctgacttgccgtaccacctacgcaccctttacacccagtaattccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggttccccccattgtgcaatattcccca

SEQ ID NO: 18 - DNA, 406 bp, Anaerotruncus sp.
  source 1..406: mol_type = other DNA
cctgtttgctacccacgctttcgagcctcagcgtcagttacagcccagtaggccgccttcgccactggtgttcctcctaatatctacgcatttcaccgctacactaggaattccgcctacctctactgcactcaagagtggcagttttgaacgcgactatcagttgagccgatagtttaaacattcaacttgcctccccgcctacgctccctttacacccagtaattccggacaacgcttgccacctacgtattaccgcggctgctggcacgtagttagccgtggcttcctctttaggtaccgtcatttttttcgtccctaaagacagaggtttacaatccgaaaaccgtcttccctcacgcggcgtcgctgcatcagggttccccccattgtgcaatatccccca

SEQ ID NO: 19 - DNA, 404 bp, Ruminococcus gnavus
  source 1..404: mol_type = other DNA
cctgtttgctccccacgctttcgagcctcaacgtcagtcatcgtccagaaagccgccttcgccactggtgttcctcctaatatctacgcatttcaccgctacactaggaattccgctttcctctccgacactctagcctgacagttccaaatgcagtcccggggttgagccccgggctttcacatctggcttgccatgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcatcgctcacgcggcgtcgctgcatcagggtttcccccattgtgcaatattcccca

SEQ ID NO: 20 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacccctccggcactcaagtatgacagtttccaatgcagtccacaggttgagcccatgcctttcacatcagacttgccacaccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattcccca

SEQ ID NO: 21 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggcactcgagtatcacagtttccaatgcagtccaggggttgagcccccgcctttcacatcagacttgcaacaccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 22 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggtactcaagatcaacagtttccaatgcagtccgggggttgagcccccgcctttcacatcagacttgctgctccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 23 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggcactctagaaaaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 24 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggcactccagatggacagtttccaaagcagtccaggggttgagcccctgcctttcacttcagacttgcccgtccgtctacgctccctttacacccagtaaatccggataacgcttgccccctacgtattaccgcggctgctggcacgtagttagccggggcttcttagtcaggtaccgtcattgtcttccctgctgatagaagtttacataccgagatacttcttccttcacgcggcgtcgctgcatcagggtttcccccattgtgcaatattcccta

SEQ ID NO: 25 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacccctccggtactcaagactgacagtttccaatgcagtccaggggttgagcccctgcctttcacatcagacttgccattccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 26 - DNA, 404 bp, Blautia sp.
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggcactcaagatggacagtttccaatgcagtcccggggttaagccccgggctttcacatcagacttgcccgtccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagagtttcctccattgtgcaatattcccca

SEQ ID NO: 27 - DNA, 405 bp, Butyricicoccus sp.
  source 1..405: mol_type = other DNA
cctgtttgctccccacgctttcgcgcctcagcgtcagttaatgtccagcaggccgccttcgccactggtgttcctccgtatatctacgcatttcaccgctacacacggaattccgcctgcctctccatcactcaagaccagcagttttgaaagcagttcgggggttaagcccccggatttcactcccaacttgccggcccgcctgcgcgccctttacacccagtaaatccggataacgcttgctccctacgtattaccgcggctgctggcacgtagttagccggagcttattcttcaggtaccgtcattcttcgtccctgattaaagatctttacaacccgaaggccttcttcaatcacgcggcgttgctgcgtcagggtttcccccattgcgcaatattcccca

SEQ ID NO: 28 - DNA, 428 bp, Catenibacterium sp.
  source 1..428: mol_type = other DNA
cctatttgctccccacgctttcgggaatgagcgtcagttgcggcccagaccgtcgccttcgccactggtgttcctccatatatctacgcatttcaccgctacacatggaattccacgatcctcttccgcactctagccgcccggtttccatggcttaccgaagtttagcttcggtctttcaccacagacccttgcggccgcctgctccctctttacgcccaatgattccggataacgctcgccacctacgtattaccgcggctgctggcacgtagttagccgtggctttctggtaaagtaccgtcactcgcataccatttcctgtatgcgccgttcttcctttacaacagagctttacgacccgaaggccttcttcgctcacgcggcgttgctcggtcagggtttcccccattgccgaaaattcccta

SEQ ID NO: 29 - DNA, 404 bp, Clostridium sp.
  source 1..404: mol_type = other DNA
cctgtttgctccccacgctttcgagcctcagcgtcagttacagtccagagagtcgccttcgccactggtgttcttcctaatctctacgcatttcaccgctacactaggaattccactctcctctcctgcactctagataatcagtttggaatgcagcccccaggttgagcctgagtatttcacatcccacttaattatccgcctacgctccctttacgcccaataattccggataacgctcgccacctacgtattaccgcggctgctggcacgtagttagccgtggcttcctccttgggtaccgtcattatcgtccccaaagacagagctttacaatccgaagaccgtcatcactcacgcggcgttgctgcatcagggtttcccccattgtgcaatattcccca

SEQ ID NO: 30 - DNA, 404 bp, Faecalibacterium sp.
  source 1..404: mol_type = other DNA
cctgtttgctacccacactttcgagcctcagcgtcagttggtgcccagtaggccgccttcgccactggtgttcctcccgatatctacgcattccaccgctacaccgggaattccgcctacctctgcactactcaagaaaaacagttttgaaagcagtttatgggttgagcccatagatttcacttccaacttgtcttcccgcctgcgctccctttacacccagtaattccggacaacgcttgtgacctacgttttaccgcggctgctggcacgtagttagccgtcacttccttgttgggtaccgtcattatcttccccaacaacaggagtttacaatccgaagaccttcttcctccacgcggcgtcgctgcatcagggtttcccccattgtgcaatattcccca

SEQ ID NO: 31 - DNA, 429 bp, unidentified
  misc_feature 1..429: note = Description of Unknown: Holdemanella sp. sequence
  source 1..429: mol_type = other DNA
cctatttgctccccacgctttcgtgcttcagtgtcagaatccagaccagacggccgccttcgccaccggtgttcttccatatatctacgcattttaccgctacacatggagttccgccgtcctcttctgttctctagctgatcagtttccagagcaagtacgggttgagcccataccttttactccagacttgatctgccacctacgcaccctttacgcccaatcattccggataacgctcgccacctacgtattaccgcggctgctggcacgtagttagccgtgactttctggtaagataccatcactcactcatcattccctatgagtgccgtttttctcttacaacagagctttacgatccgaagaccttcctcactcacgcggcattgctcgttcagggttccccccattgacgaaaattcccta

SEQ ID NO: 32 - DNA, 405 bp, Lachnoclostridium sp.
  source 1..405: mol_type = other DNA
cctgtttgctccccacgctttcgagcctcaacgtcagttactgtccagtaagccgccttcgccactggtgttcctcctaatatctacgcatttcaccgctacactaggaattccacttacctctccagcactctagcaacacagtttccaaagcagtcccagggttgagccctgggctttcacttcagacttgcatcgccgtctacgctccctttacacccagtaaatccggataacgcttgccccctacgtattaccgcggctgctggcacgtagttagccggggcttcttagtcaggtaccgtctttcttcttccctgctgatagagctttacataccgaaatacttcttcactcacgcggcgtcgctgcatcagggtttcccccattgtgcaatattcccca

SEQ ID NO: 33 - DNA, 404 bp, Lachnoclostridium sp.
  source 1..404: mol_type = other DNA
cctgtttgctccccacgctttcgagcctcaacgtcagttactgtccagtaagccgccttcgccactggtgttcctcctaatatctacgcatttcaccgctacactaggaattccacttacctctccagcactcaagctacacagtttccaaagcagtcccggggttgagccccgggctttcacttcagacttgcacagccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 34 - DNA, 404 bp, Lachnoclostridium sp.
  source 1..404: mol_type = other DNA
cctgtttgctccccacgctttcgagcctcaacgtcagttatcgtccagtaagccgccttcgccactgatgttcctcctaatatctacgcatttcaccgctacactaggaattccgcttacctctccgacactctagaagcacagtttccaaagcagtcacggggttgagccccgggctttcacttcagacttgcacttccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 35 - DNA, 404 bp, Lachnoclostridium sp.
  source 1..404: mol_type = other DNA
cctgtttgctccccacgctttcgagcctcaacgtcagttactgtccagtaagccgccttcgccactggtgttcctcctaatatctacgcatttcaccgctacactaggaattccgcttacctctccagcactccagcttaacagtttccaaagcagtcccggggttgagccccgggctttcacttcagacttgctaagccgtctacgctccctttacacccagtaaatccggataacgcttgccccctacgtattaccgcggctgctggcacgtagttagccggggcttcttagtcaggtaccgtcattctcttccctgctgatagagctttacataccgaaatacttcttcactcacgcggcgtcgctgcatcagggtttcccccattgtgcaatattcccca

SEQ ID NO: 36 - DNA, 404 bp, Lachnoclostridium sp.
  source 1..404: mol_type = other DNA
cctgtttgctccccacgctttcgagcctcaacgtcagtcatcgtccagtaagccgccttcgccactggtgttcctcctaatatctacgcatttcaccgctacactaggaattccacttacctctccgacactctagaaaaacagtttccaatgcagtcccggggttgagccccgggttttcacatcagacttgcctctccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcatcgctcacgcggcgtcgctgcatcagggtttcccccattgtgcaatattcccca

SEQ ID NO: 37 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagttatcgtccagcaagccgccttcgccactggtgttcctcctaatatctacgcatttcaccgctacactaggaattccgcttgcctctccgacactccagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 38 - DNA, 404 bp, Eubacterium sp.
  source 1..404: mol_type = other DNA
cctgtttgctccccacgctttcgtgcctcagtgtcagtaacagtccagcaggccgccttcgccactggtgttcctcctaatatctacgcatttcaccgctacactaggaattccgcctgcctctcctgtactctagccgagcagtttcaaatgcagctccggggttgagccccggcctttcacatctgacttgcactgccacctacgcaccctttacacccagtaaatccggataacgcttgctccatacgtattaccgcggctgctggcacgtatttagccggagcttcttagtcaggtaccgtcattatcttccctgctgatagagctttacataccgaaatacttcttcactcacgcggcgttgctgcatcagggtttcccccattgtgcaatattcccca

SEQ ID NO: 39 - DNA, 404 bp, Ruminococcus sp.
  source 1..404: mol_type = other DNA
cctgtttgctccccacgctttcgagcctcaacgtcagttatcgtccagtgagccgccttcgccactggtgttcctcctaatatctacgcatttcaccgctacactaggaattccactcacctctccgacactctagttatacagtttccaatgcaggtccagggttaagccctggaatttcacatcagacttgcacaaccgtctacgctccctttacacccagtaaatccggataacgcttgctccatacgtattaccgcggctgctggcacgtatttagccggagcttcttagtcaggtaccgtcattatcttccctgctgatagagctttacataccgaaatacttcttcgctcacgcggcgtcgctgcatcagggtttcccccattgtgcaatattcccca

SEQ ID NO: 40 - DNA, 404 bp, Ruminococcus sp.
  source 1..404: mol_type = other DNA
cctgtttgctccccacgctttcgagcctcaacgtcagttatcgtccagtaagccgccttcgccactggtgttcctcctaatatctacgcatttcaccgctacactaggaattccacttacccctccgacactctagactgacagtttccaatgcagtcccggggttgagccccgggttttcacatcagacttgccagtccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcgctcacgcggcgtcgctgcatcagggtttcccccattgtgcaatattcccca

SEQ ID NO: 41 - DNA, 403 bp, unidentified
  misc_feature 1..403: note = Description of Unknown: Lachnospiraceae family FCS020 group bacterium sequence
  source 1..403: mol_type = other DNA
cctgtttgctccccacgctttcgtgcctcagcgtcagttacagtccagaaagccgccttcgctactggtgttcctcccaatatctacgcatttcaccgctacactgggaattccgctttcctctcctgcactcaagtcagacagtatcaggagcttactacggttgagccgtagcctttaactcctgacttgaaagaccgcctacgcaccctttacgcccagtaaatccggataacgctagccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 42 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagttactgtccagtaagccgccttcgccactggtgttcctcctaatatctacgcatttcaccgctacactaggaattccgcttacctctccagtactctagccgcacagtttccaatgcagtcccggggttgagccccgggctttcacatcagacttgcaccgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 43 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagttaccgtccagtaagccgccttcgccactggtgttcctcctaatatctacgcatttcaccgctacactaggaattccgcttacctctccggcactccagcagaacagtttccaatgcaggtccggggttgagccccggaatttcacatcagacttgctccgccgtctacgctccctttacacccagtaaatccggataacgcttgccccctacgtattaccgcggctgctggcacgtagttagccggggcttcttagtcaggtaccgtcattatcttccctgctgatagagctttacataccgaaatacttcttcgctcacgcggcgtcgctgcatcagggtttcccccattgtgcaatattcccca

SEQ ID NO: 44 - DNA, 404 bp, unidentified
  misc_feature 1..404: note = Description of Unknown: Lachnospiraceae family NK4A136 group bacterium sequence
  source 1..404: mol_type = other DNA
cctgtttgctccccacgctttcgagcctcaacgtcagttaccgtccagtaagccgccttcgccactggtgttcttcctaatatctacgcatttcaccgctacactaggaattccgcttacctctccggcactccagctaaacagtttccaaagcagtcccggcgttaagcaccgggctttcacttcagacttgccttgccgtctacactccctttacacccagtaaatccggataacgcttgccccctacgtattaccgcggctgctggcacgtagttagccggggcttcttattcaggtaccgtcactttcttccctgctgatagagctttacataccgaaatacttcttcactcacgcggcgtcgctgcatcagggtttcccccattgtgcaatattcccca

SEQ ID NO: 45 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagttactgtccagtaagccgccttcgccactggtgttcctcctaatatctacgcatttcaccgctacactaggaattccacttacctctccagcactctagcagaacagtttccaaagcagtcccggggttgagccccgggctttcacttcagacttgctccgccgtctacgctccctttacacccagtaaatccggataacgcttgccccctacgtattaccgcggctgctggcacgtagttagccggggcttcttagtcaggtaccgtcattttcttccctgctgatagagctttacataccgagatacttcttcactcacgcggcgtcgctgcatcagggtttcccccattgtgcaatatccccca

SEQ ID NO: 46 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagttactgtccagcaagccgccttcgccactggtgttcctcctaatatctacgcatttcaccgctacactaggaattccgcttgcctctccagcactccagcccaacagtttccaaagcagttcccgggttgagcccggggatttcacttcagacttgctgtgccgtctacgctccctttacacccagtaaatccggataacgcttgccccctacgtattaccgcggctgctggcacgtagttagccggggcttcttagtcaggtaccgtcactttcttccctgctgatagagctttacataccgaaatacttcttcactcacgcggcgttgctgcatcagggtttcccccattgtgcaatattcccca

SEQ ID NO: 47 - DNA, 429 bp, Lactobacillus sp.
  source 1..429: mol_type = other DNA
cctgtttgctacccacgctttcgaacctcagcgtcagttacagaccagagagccgctttcgccactggtgttcttccatatatctacgcatttcaccgctacacatggagttccactctcctcttctgcactcaagtctcccagtttccaatgcactactccggttaagccgaaggctttcacatcagacttaaaagaccgcctgcgttccctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattcccta

SEQ ID NO: 48 - DNA, 429 bp, Lactobacillus sp.
  source 1..429: mol_type = other DNA
cctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattcccta

SEQ ID NO: 49 - DNA, 429 bp, Lactobacillus sp.
  source 1..429: mol_type = other DNA
cctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactacttcggttaagccgaaggctttcacatcagacttaaaagaccgcctgcgttccctttacgcccaataaatccggacaacgctcgccacctacgtattaccgcggctgctggcacgtagttagccgtggctttctggtcagataccgtcaatacgtgaacagttactctcacgcacgttcttctctgacaacagaattttacgacccgaaggccttcttcattcacgcggcgttgctccatcagactttcgtccattgtggaagattcccta

SEQ ID NO: 50 - DNA, 429 bp, Megamonas sp.
  source 1..429: mol_type = other DNA
cccgttcgctaccctagctttcgagcctcagcgtcagttacagtccagaaagccgccttcgccactggtgttcttcccaatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 51 - DNA, 429 bp, Megamonas sp.
  source 1..429: mol_type = other DNA
cccgttcgctaccctagctttcgagcctcagcgtcagttacagtccagaaagccgccttcgccactggtgttcttcccaatatctacgcatttcaccgctacactgggaattccgctttcctctcctgcactcaagaaaatcagttcggaccccatcacgaggttgagccccgcacttttaagatccgcttaatttcccgcctgcgctccctttacgcccaataattccggacaacgcttgccacctacgtattaccgcggctgctggcacgtagttagccgtggcttcctcgttaagtaccgtcaaatacttactgtattataataagcatccttcgcccttaacaacagaactttacgatccgaagaccttcttcgttcacgcggcgttgctccgtcaggctttcgcccattgcggaagattcccca

SEQ ID NO: 52 - DNA, 429 bp, Megasphaera sp.
  source 1..429: mol_type = other DNA
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcgttcacgcggcgttgctccgtcagactttcgtccattgcggaagattcccca

SEQ ID NO: 53 - DNA, 429 bp, Megasphaera sp.
  source 1..429: mol_type = other DNA
cctgtttgctccccacgctttcgagcctcaacgtcagttaccgtccagtaagccgccttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcgttcacgcggcgttgctccgtcagactttcgtccattgcggaagattcccca

SEQ ID NO: 54 - DNA, 429 bp, Megasphaera sp.
  source 1..429: mol_type = other DNA
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcccgcacgcggcgtcgctgcatcaggcttgcgcccattgtgcaatattcccca

SEQ ID NO: 55 - DNA, 429 bp, Megasphaera sp.
  source 1..429: mol_type = other DNA
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcactcgtaacgggtattgaccgctacgccattcgtcccgtataacagaactttacaacccgaaggccgtcatcgttcacgcggcgttgctccgtcagactttcgtccattgcggaagattcccca

SEQ ID NO: 56 - DNA, 429 bp, Megasphaera sp.
  source 1..429: mol_type = other DNA
cctgtttgctccccacgctttcgagcctcaacgtcagttaccgtccagtaagccgccttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcactcgtaacgggtattgaccgctacgccattcgtcccgtataacagaactttacaacccgaaggccgtcatcgttcacgcggcgttgctccgtcagactttcgtccattgcggaagattcccca

SEQ ID NO: 57 - DNA, 429 bp, Megasphaera sp.
  source 1..429: mol_type = other DNA
cccgttcgctcccctggctttcgtgcctcagcgtcagttgtcgtccagaaagccgctttcgccactggtattcctcctaatatctacgcatttcaccgctacactaggaattctgctttcctctccgatactccagtctcccagtttccgtcccctcacggggttgagccccgcacttttaagacagacttaagagaccgcctgcgcaccctttacgcccaataattccggacaacgcttgccacctacgtattaccgcggctgctggcacgtagttagccgtggctttctcttacggtaccgtcacaacaggatggtattagcatcctgcccgttcgtcccgtataacagagctttacaacccgaaggccgtcttcactcacgcggcgttgctccgtcagactttcgtccattgcggaagattcccca

SEQ ID NO: 58 - DNA, 403 bp, unidentified
  misc_feature 1..403: note = Description of Unknown: Romboutsia sp. sequence
  source 1..403: mol_type = other DNA
cctgtttgctccccacgctttcgtgcctcagcgtcagttacagtccagagagccgccttcgcaactggtgttcctcctaatatctacgcatttcaccgctacactaggaattccactctcctctcctgcactcaagtcttacagtttcaaaagcttactacggttgagccgtagcctttcacttctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 59 - DNA, 406 bp, Roseburia sp.
  source 1..406: mol_type = other DNA
cctgtttgctccccacgctttcgagcctcagcgtcagtcatcgtccagtaagccgccttcgccactggtgttcctcctaatatctacgcatttcaccgctacactaggaattccacttacccctccgacactctagtccgacagtttccaatgcagtaccggggttgagccccgggctttcacatcagacttgccgtaccgcctgcgctccctttacacccagtaaatccggataacgcttgcaccatacgtattaccgcggctgctggcacgtatttagccggtgcttcttagtcaggtaccgtcatttcttcttccctgctgatagagctttacataccgaaatacttcttcgctcacgcggcgtcgctgcatcagggtttcccccattgtgcaatattcccca

SEQ ID NO: 60 - DNA, 404 bp, unidentified
  misc_feature 1..404: note = Description of Unknown: Ruminococcaceae family bacterium sequence
  source 1..404: mol_type = other DNA
cctgtttgctacccatgctttcgagcctcagcgtcagttagtgcccagtaggccgccttcgccactggtgttcctcccgatatctacgcattccaccgctacaccgggaattccgcctacctctacaccactcaagactgacagttttgaaagcaatttatgggttgagcccatagatttcactcccaacttgccaatccgcctgcgctccctttacacccagtaattccggacaacgcttgcaccctacgttttaccgcggctgctggcacgtagttagccggtgcttcctcccagggtaccgtcattatcgtcccctgggacaggagtttacaatccgaagaccttcttcctccacgcggcgtcgctgcatcagggtttcccccattgtgcaatattcccca

SEQ ID NO: 61 - DNA, 406 bp, unidentified
  misc_feature 1..406: note = Description of Unknown: Ruminococcaceae family bacterium sequence
  source 1..406: mol_type = other DNA
cctgtttgctccccacgctttcgcgcctcaccgtcagttgtcgtccagtaatccgccttcgccactggtgttcttccttatatctacgcatttcaccgctacacaaggaattccgattacctctccgatactcaagaccaacagtttcaaatgcagttcacgggttaagcccgtggatttcacatctgacttgccggcccggctgcacgccctttacacccagtaaatccggacaacgcttgccacctacgtattaccgcggctgctggcacgtagttagccgtggcttattcgtcaggtaccgtcttctgctcttccctgacaaaagaagtttacaacccgaaagccttcttccttcacgcggcgttgctgggtcagacttgcgtccattgcccaatattcccca

SEQ ID NO: 62 - DNA, 404 bp, unidentified
  misc_feature 1..404: note = Description of Unknown: Sarcina sp. sequence
  source 1..404: mol_type = other DNA
cctgtttgctccccacgctttcgagcctcagcgtcagttacagtccagagaatcgccttcgccactggtgttcttcctaatctctacgcatttcaccgctacactaggaattccattctcctctcctgcactctagatatccagtttggaatgcagctcccaagttaagctcgggtatttcacatcccacttaaatatccgcctacgctccctttacgcccagtaaatccggataacgcttgccacctacgtattaccgcggctgctggcacgtagttagccgtggcttcctccttaggtaccgtcattatcgtccctaaagacagagctttacaatccgaagaccttcatcactcacgcggcgttgctgcatcagggtttcccccattgtgcaatattcccca

SEQ ID NO: 63 - DNA, 404 bp, unidentified
  misc_feature 1..404: note = Description of Unknown: Sellimonas sp. sequence
  source 1..404: mol_type = other DNA
cctgtttgctccccacgctttcgagcctcaacgtcagtcaccgtccagtaagccgccttcgccactggtgttcctcctaatatctacgcatttcaccgctacactaggaattccgcttacctctccggcactccagaattgcagtttccaaagcagtcccagggttgagccctgggttttcactccagacttgcctctccgtctacgctccctttacacccagtaaatccggataacgcttgcaccatacgtattaccgcggctgctggcacgtatttagccggtgcttcttagtcaggtaccgtcagtttcttccctgctgatagaagtttacataccgaaatacttcttccttcacgcggcgtcgctgcatcagggtttcccccattgtgcaatattcccca

SEQ ID NO: 64 - DNA, 404 bp, Subdoligranulum sp.
  source 1..404: mol_type = other DNA
cctgtttgctacccatgctttcgagcctcagcgtcagttggtgcccagtaggccgccttcgccactggtgttcctcccgatatctacgcattccaccgctacaccgggaattccgcctacctctgcactactcaaggccagcagttttgaaagcaatttatgggttgagcccatagatttcacttccaacttgccggtccgcctgcgctccctttacacccagtaattccggacaacgcttgccacctacgttttaccgcggctgctggcacgtagttagccgtggctttcttgtacggtaccgtcattatcgtcccgtacgacaggagtttacaatccgaaaaccttcttcctccacgcggcgtcgctgcatcagggtttcccccattgtgcaatatccccca

SEQ ID NO: 65 - DNA, 406 bp, Subdoligranulum sp.
  source 1..406: mol_type = other DNA
cctgtttgctacccatgctttcgagcctcagcgtcagttggtgcccagtaggtcgccttcgccactggtgttcctcccgatatctacgcattccaccgctacaccgggaattccacctacctctgcactactcaaggccagcagttttgaaagcaatttatgggttgagcccatagatttcactcccaacttgccggtccgcctgcgctccctttacacccagtaattccggacaacgcttgcaccctacgttttaccgcggctgctggcacgtagttagccggtgctttcttgttaggtaccgtcagatattcgtccctaacgacaggagtttacaatccgaaaaccttcttcctccacgcggcgtcgctgcatcagggtttcccccattgtgcaatatccccca

SEQ ID NO: 66 - DNA, 429 bp, Turicibacter sp.
  source 1..429: mol_type = other DNA
cctgtttgctccccacgctttcgcgcctcagtgtcagttacagaccaggaagccgccttcgccactggtgttcctccatatctctacgcatttcaccgctacacatggaattccacttccctcttctgcactcaagtcgaccagtttccaatgaccctccacggttaagccgtgggctttcacatcagacttaatcaaccacctgcgcgctctttacgcccaataattccggataacgctcgccacctacgtattaccgcggctgctggcacgtagttagccgtggctttctcataaggtaccgtcacactctagccatttcctactaaagtcgttcttcccttataacagaattttacaacccgaaggccttcatcattcacgcggcgttgctcggtcaggctttcgcccattgccgaagattcccta

SEQ ID NO: 67 - DNA, 407 bp, unidentified
  misc_feature 1..407: note = Description of Unknown: Ruminococcaceae family UCG-009 bacterium sequence
  source 1..407: mol_type = other DNA
cctgtttgctccccacgctttcgcgcctcagcgtcagttaatgtccagcaggccgccttcgccactggtgttcctcccgatatctacgcattccaccgctacaccgggaattccgcctgcctctccatcactcaagaactacagtttcaaatgcagtctatgggttgagcccatagttttcacatctgacttgcaatcccgcctgctcgccctttacacccagtaaatccggataacgcttgccacctacgtattaccgcggctgctggcacgtagttagccgtggcttattcttacggtaccgtcatttgtttcgtcccgtacaaaagaagtttacaatccgaaaaccttcatccttcacgcggcattgctgcatcagggtttcccccattgtgcaatattcccca

SEQ ID NO: 68 - DNA, 404 bp, unidentified
  misc_feature 1..404: note = Description of Unknown: Uncultured Blautia bacterium sequence
  source 1..404: mol_type = other DNA
cctgtttgctccccacgctttcgagcctcaacgtcagttacagtccagtaagccgccttcgccactggtgttcctcctaatatctacgcatttcaccgctacactaggaattccgcttacctctcctgcactctagcttaacagtttccaatgcagtcttggggttgagccccaagttttcacatcagacttgccatgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 69 - DNA, 404 bp, unidentified
  misc_feature 1..404: note = Description of Unknown: Lachnospiraceae family bacterium sequence
  source 1..404: mol_type = other DNA
cctgtttgctccccacgctttcgagcctcaacgtcagtcatcgtccagcaagccgccttcgccactggtgttcctcctaatatctacgcatttcaccgctacactaggaattccacttgcctctccgacactctagctctacagttccaaatgcagtcccggggttgagccccgggctttcacatctggcttgcaatgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 70 - DNA, 406 bp, Anaerobiospirillum succiniciproducens
  source 1..406: mol_type = other DNA
cctgtttgctccccacgctttcgcacctcagcgtcagtatttgcccaggagaccgccttcgcctctggtgttctttcagatatctacgcattccaccgctacacctgaaattccatctccctctgcaatactctagaaaggcagtttagaatgaccaacccaggttgagcccgggtatttcacatcctacttacctttccgcctacatgccctttacgcccagttattccgattaacgcttgcaccctccgtattaccgcggctgctggcacggagttagccggtgcttcttctgtgggtaacgtcagtgtttcctccccactgaaagtgctttacaacccgaaggccttcttcacacacgcggcatggctgcatcagggtttcccccattgtgcaatattcccta

SEQ ID NO: 71 - DNA, 406 bp, Anaerobiospirillum succiniciproducens
  source 1..406: mol_type = other DNA
cctgtttgctccccacgctttcgcacctcagcgtcagtatttgcccaggagaccgccttcgcctctggtgttctttcagatatctacgcattccaccgctacacctgaaattccatctccctctgcaatactctagaaaggtagttcaaaatgaccaacccaggttgagcccaggtctttcacatcttgcttgcctttccgcctacatgccctttacgcccagttattccgattaacgcttgcaccctccgtattaccgcggctgctggcacggagttagccggtgcttcttctgtgggtaacgtcagtatttcctccccactgaaagtgctttacaacccgaaggccttcttcacacacgcggcatggctgcatcagggtttcccccattgtgcaatattcccta

SEQ ID NO: 72 - DNA, 404 bp, Succinivibrio sp.
  source 1..404: mol_type = other DNA
cctgtttgctacccacgctttcgcatatgagcgtcagtatttgcccaggagactgccttcgcctttggtgttcttccagatctctacgcattccaccgctacacctggaattccgtctccccctgcaatactctagtcccacagtttagagtgcaattccgaggttgagccccgggctttcacaccctacttgcagaaccgcctgcatgccctttacgcccagttattccgattaacgcttgcaccctccgtattaccgcggctgctggcacggagttagccggtgcttcttctgagggtaacgtcattttcctcccctctgaaagtgctttacaacccgaaggccttcttcacacacgcggcatggctgcatcagggttccccccattgtgcaatattcccta

SEQ ID NO: 73 - DNA, 407 bp, Succinivibrio sp.
  source 1..407: mol_type = other DNA
cctgtttgctacccacgctttcgcatatgagcgtcagtatttgcccaggagactgccttcgcctttggtgttcttccagatatctacgcattccaccgctacacctggaattccgtctccccctgcaatactctagtccaccagtttagtatgctgttcccaggttgagcccggggatttcacatcctacttgatgaaccgcctgcatgccctttacgcccagttattccgattaacgcttgcaccctccgtattaccgcggctgctggcacggagttagccggtgcttcttctgagggtaacgtcattttcttcctcccctccgaaagtgctttacaacccgaaggccttcttcacacacgcggcatggctgcatcagagtttcctccattgtgcaatattcccta